(19) United States
(12) Patent Application Publication
(10) Pub. No.: US 2026/0053333 A1
(43) Pub. Date: Feb. 26, 2026

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Rito MURASE, Kanagawa (JP)

(73) Assignee: FUJIFILM Corporation, Tokyo (JP)

(21) Appl. No.: 19/304,538

(22) Filed: Aug. 19, 2025

(30) Foreign Application Priority Data
Aug. 23, 2024 (JP) 2024-143371

(51) Int. Cl. A61B 1/00; A61B 1/31; G06T 7/11

(52) U.S. Cl. CPC A61B 1/000094; A61B 1/000096; A61B 1/0005; G06T 7/11; A61B 1/31; G06T 2207/10068

(57) ABSTRACT

A processor of a medical support device is configured to: acquire a medical image; and selectively output a plurality of pieces of lumen specification information. The medical image is classified into a first medical image and a second medical image. The plurality of pieces of lumen specification information include first lumen specification information and second lumen specification information. The first lumen specification information is generated based on information obtained from a trained model in a case in which the first medical image is input to the trained model, and is information capable of specifying a first existence position. The second lumen specification information is generated based on information obtained from a time-series model in a case in which time-series information is input to the time-series model, and is information capable of specifying a second existence position.

### CROSS-REFERENCE TO RELATED APPLICATIONS

This application claims priority under 35 USC 119 from Japanese Patent Application No. 2024-143371 filed on Aug. 23, 2024, the disclosure of which is incorporated by reference herein.

### BACKGROUND

### 1. Technical Field

The present disclosure relates to a medical support device, an endoscope system, a medical support method, and a program.

### 2. Related Art

WO2020/194472A discloses a movement support system comprising a plural-operation information calculation unit and a presentation information generation unit. In the movement support system disclosed in WO2020/194472A, the plural-operation information calculation unit calculates a plurality of pieces of operation information indicating a plurality of operations different in time, which correspond to a plurality of operation target scenes that are scenes in which the plurality of operations different in time are required, based on captured images acquired by an imaging unit arranged in an insertion part. The presentation information generation unit generates presentation information for the insertion part based on the plurality of pieces of operation information calculated by the plural-operation information calculation unit.

In addition, the plural-operation information calculation unit calculates the plurality of pieces of operation information via a trained model that has been trained through machine learning using, as inputs, the captured images corresponding to the plurality of operation target scenes that are scenes in which the plurality of operations different in time are required.

Further, the plural-operation information calculation unit calculates a likelihood of the plurality of pieces of operation information, and presents information indicating that the certainty of the plurality of pieces of operation information is low in a case in which the likelihood is lower than a threshold value set in advance for the likelihood of the plurality of pieces of operation information.

### SUMMARY

One embodiment according to the present disclosure provides a medical support device, an endoscope system, a medical support method, and a program that enable a user or the like to ascertain an existence position of a lumen, which is shown in a plurality of medical images arranged in time series, in each medical image without omission.

A first aspect according to the present disclosure relates to a medical support device comprising: a processor configured to: acquire a medical image generated by imaging an inside of a luminal organ; and selectively output a plurality of pieces of lumen specification information that are information capable of specifying an existence position of a lumen, which is shown in the medical image, in the medical image, in which the medical image is classified into a first medical image and a second medical image obtained later than the first medical image, the plurality of pieces of lumen specification information include first lumen specification information and second lumen specification information, the first lumen specification information is generated based on information obtained from a trained model in a case in which the first medical image is input to the trained model, and is information capable of specifying a first existence position that is the existence position of the lumen, which is shown in the first medical image, in the first medical image, and the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image.

A second aspect according to the present disclosure relates to the medical support device according to the first aspect, in which the processor is configured to output the second lumen specification information in a case in which the second medical image is an image that satisfies an erroneous specification triggering condition that triggers erroneous specification of the second existence position by the trained model.

A third aspect according to the present disclosure relates to the medical support device according to the second aspect, in which the plurality of pieces of lumen specification information include third lumen specification information, the processor is configured to output the third lumen specification information in a case in which the second medical image is an image that does not satisfy the erroneous specification triggering condition, and the third lumen specification information is generated based on information obtained from the trained model in a case in which the second medical image is input to the trained model, and is information capable of specifying a third existence position that is the existence position of the lumen, which is shown in the second medical image, in the second medical image.

A fourth aspect according to the present disclosure relates to the medical support device according to the first aspect, in which the plurality of pieces of lumen specification information include third lumen specification information, the processor is configured to output the third lumen specification information in a case in which the second medical image is an image that does not satisfy an erroneous specification triggering condition that triggers erroneous specification of the second existence position by the trained model, and the third lumen specification information is generated based on information obtained from the trained model in a case in which the second medical image is input to the trained model, and is information capable of specifying a third existence position that is the existence position of the lumen, which is shown in the second medical image, in the second medical image.

A fifth aspect according to the present disclosure relates to the medical support device according to any one of the second to fourth aspects, in which the erroneous specification triggering condition includes a first condition in which at least a part of the lumen is not shown in the second medical image, a second condition in which at least one dark portion different from the lumen is shown in the second medical image, a third condition in which an obstruction that obstructs the lumen is shown in the second medical image, a fourth condition in which an image quality triggers the erroneous specification, and/or a fifth condition in which a portion of the luminal organ that triggers the erroneous specification is shown in the second medical image.

A sixth aspect according to the present disclosure relates to the medical support device according to any one of the second to fifth aspects, in which the trained model generates a confidence level indicating that the lumen is shown for each of a plurality of divided regions obtained by dividing the second medical image or an image corresponding to the second medical image, in a case in which the second medical image is input to the trained model, and the erroneous specification triggering condition includes a confidence level condition related to reliability of the confidence level for each of the plurality of divided regions.

A seventh aspect according to the present disclosure relates to the medical support device according to the sixth aspect, in which the confidence level condition includes a condition in which the confidence level for each of the plurality of divided regions is equal to or greater than a second threshold value, which is less than a first threshold value, and less than the first threshold value.

An eighth aspect according to the present disclosure relates to the medical support device according to any one of the first to seventh aspects, in which the first medical image or an image corresponding to the first medical image has a plurality of partition regions obtained by partitioning the first medical image or the image corresponding to the first medical image along a circumferential direction, the trained model generates a plurality of confidence levels corresponding to the plurality of partition regions and indicating that the lumen is shown for each of the plurality of partition regions, in a case in which the first medical image or the image corresponding to the first medical image is input to the trained model, the first lumen specification information is information capable of specifying the first existence position more precisely than the partition regions in the first medical image or the image corresponding to the first medical image, and the first lumen specification information is generated based on the plurality of partition regions and the plurality of confidence levels.

A ninth aspect according to the present disclosure relates to the medical support device according to the eighth aspect, in which the first lumen specification information is information capable of specifying the first existence position with a higher resolution than the plurality of partition regions along the circumferential direction.

A tenth aspect according to the present disclosure relates to the medical support device according to the eighth or ninth aspect, in which a direction from a reference position of the first medical image or the image corresponding to the first medical image to an existence position of each of the plurality of partition regions is determined by a plurality of first vectors, a direction from the reference position to a lumen existence region including the first existence position is determined by a second vector, the second vector is a sum of at least two third vectors obtained by adding the confidence level, as a weight, to at least two first vectors among the plurality of first vectors, and the first lumen specification information is information determined based on the second vector.

An eleventh aspect according to the present disclosure relates to the medical support device according to any one of the first to tenth aspects, in which the second medical image or an image corresponding to the second medical image has a central region of the second medical image and a plurality of partition regions obtained by radially partitioning surroundings of the central region in the second medical image, the trained model generates a confidence level indicating that the lumen is shown for the central region and each of the plurality of partition regions, in a case in which the second medical image is input to the trained model, and in a case in which the confidence level for each of the central region and the plurality of partition regions is equal to or greater than a third threshold value or the confidence level for each of the central region and the plurality of partition regions is less than a fourth threshold value less than the third threshold value, information based on a processing result obtained by the trained model is output preferentially over information based on a processing result obtained by the time-series model.

A twelfth aspect according to the present disclosure relates to the medical support device according to any one of the first to eleventh aspects, in which the trained model generates a confidence level indicating that the lumen is shown in the first medical image, in a case in which the first medical image is input to the trained model, and in a case in which the confidence level is equal to or less than a reference value, the output of the lumen specification information, which is output before the confidence level equal to or less than the reference value is generated, is continued.

A thirteenth aspect according to the present disclosure relates to the medical support device according to the twelfth aspect, in which, in a case in which a state in which the confidence level generated by the trained model is equal to or less than the reference value continues for a predetermined period each time the first medical image is input to the trained model, an output level of the lumen specification information, which is output before the predetermined period starts, gradually decreases throughout the predetermined period.

A fourteenth aspect according to the present disclosure relates to the medical support device according to any one of the first to thirteenth aspects, in which the output of the lumen specification information is carried out by displaying the lumen specification information on a screen.

A fifteenth aspect according to the present disclosure relates to the medical support device according to the fourteenth aspect, in which the medical image and/or an image corresponding to the medical image is displayed on the screen, and the lumen specification information is displayed on the screen in a state of being comparable with the medical image or the image corresponding to the medical image displayed on the screen.

A sixteenth aspect according to the present disclosure relates to the medical support device according to the fifteenth aspect, in which the lumen specification information is displayed in a superimposed manner on the medical image and/or the image corresponding to the medical image.

A seventeenth aspect according to the present disclosure relates to the medical support device according to the fifteenth or sixteenth aspect, in which the lumen specification information displayed on the screen is updated in accordance with the display of the medical image and/or the image corresponding to the medical image.

An eighteenth aspect according to the present disclosure relates to an endoscope system comprising: the medical support device according to any one of the first to seventeenth aspects; and an endoscope, in which the medical image is generated by imaging the inside of the luminal organ with the endoscope.

A nineteenth aspect according to the present disclosure relates to a medical support method comprising: acquiring a medical image generated by imaging an inside of a luminal organ; and selectively outputting a plurality of pieces of lumen specification information that are information capable of specifying an existence position of a lumen, which is shown in the medical image, in the medical image, in which the medical image is classified into a first medical image and a second medical image obtained later than the first medical image, the plurality of pieces of lumen specification information include first lumen specification information and second lumen specification information, the first lumen specification information is generated based on information obtained from a trained model in a case in which the first medical image is input to the trained model, and is information capable of specifying a first existence position that is the existence position of the lumen, which is shown in the first medical image, in the first medical image, and the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image.

A twentieth aspect according to the present disclosure relates to a program causing a computer to execute medical support processing comprising: acquiring a medical image generated by imaging an inside of a luminal organ; and selectively outputting a plurality of pieces of lumen specification information that are information capable of specifying an existence position of a lumen, which is shown in the medical image, in the medical image, in which the medical image is classified into a first medical image and a second medical image obtained later than the first medical image, the plurality of pieces of lumen specification information include first lumen specification information and second lumen specification information, the first lumen specification information is generated based on information obtained from a trained model in a case in which the first medical image is input to the trained model, and is information capable of specifying a first existence position that is the existence position of the lumen, which is shown in the first medical image, in the first medical image, and the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope system is used by a doctor;

FIG. 2 is a conceptual diagram showing an example of an overall configuration of the endoscope system;

FIG. 3 is a block diagram showing an example of a hardware configuration of an electrical system of the endoscope system;

FIG. 4 is a block diagram showing an example of main functions of a processor provided in a medical support device and an example of information stored in a storage;

FIG. 5 is a block diagram showing an example of a hardware configuration of an electrical system of an information processing device;

FIG. 6 is a conceptual diagram showing an example of an aspect in which training data is generated by the information processing device;

FIG. 7 is a conceptual diagram showing an example of an example image;

FIG. 8 is a conceptual diagram showing an example of the training data generated in a case in which a lumen is shown in a region other than a central region of the example image shown in FIG. 7;

FIG. 9 is a conceptual diagram showing an example of the training data generated in a case in which the lumen is shown in the central region of the example image shown in FIG. 7;

FIG. 10 is a conceptual diagram showing an example of processing contents in the information processing device in a case in which a lumen recognition model is generated by training a model through machine learning using the training data;

FIG. 11 is a block diagram showing an example of the hardware configuration of the electrical system of the information processing device;

FIG. 12 is a conceptual diagram showing an example of processing contents in the information processing device in a case in which a time-series model is constructed based on a time-series dataset;

FIG. 13 is a conceptual diagram showing an example of processing contents in lumen recognition processing performed by a recognition unit;

FIG. 14 is a conceptual diagram showing an example of confidence level information generated by the lumen recognition model in a case in which the lumen is shown in a frame;

FIG. 15 is a conceptual diagram showing an example of a relationship between a plurality of partition regions obtained by radially partitioning a map included in the confidence level information and a plurality of direction unit vectors added to the plurality of partition regions;

FIG. 16 is a conceptual diagram showing an example of processing contents in a case in which a lumen existence region is specified by a controller of the medical support device and a mark capable of specifying the lumen existence region is generated;

FIG. 17 is a conceptual diagram showing an example of processing contents using an N-th frame;

FIG. 18 is a conceptual diagram showing an aspect example in which, in a case in which the lumen is shown in a region other than a central region of the frame, the frame is displayed in a first display region of a screen, a mark capable of specifying a position of the lumen existence region, in which an existence position of the lumen shown in the frame is specified, in the frame is displayed in a superimposed manner on the frame, and visible information is displayed in a second display region of the screen as one auxiliary information;

FIG. 19 is a conceptual diagram showing an aspect example in which, in a case in which the lumen is shown in the central region of the frame, the frame is displayed in the first display region of the screen, the mark capable of specifying the position of the lumen existence region, in which the existence position of the lumen shown in the frame is specified, in the frame is displayed in a superimposed manner on the frame, and the visible information is displayed in the second display region of the screen as one the auxiliary information;

FIG. 20 is a conceptual diagram showing an example of processing contents in the controller in a case in which each of confidence levels added to all the partition regions of the map included in the confidence level information is equal to or less than a reference value;

FIG. 21 is a conceptual diagram showing a form example in which display of the mark is continued in a case in which the confidence level transitions to be equal to or less than the reference value;

FIG. 22 is a conceptual diagram showing an aspect example in which a display intensity of the mark gradually decreases in a case in which a state in which the confidence level is equal to or less than the reference value continues for a predetermined period;

FIG. 23 is a conceptual diagram showing an example of processing contents in lumen prediction processing performed by the recognition unit;

FIG. 24 is a conceptual diagram showing an aspect example in which the frame is displayed in the first display region of the screen and the mark is generated based on state information obtained by performing the lumen prediction processing and is displayed in a superimposed manner on the frame;

FIG. 25 is a conceptual diagram showing an example of processing contents in the recognition unit in a case in which a within-reliability-range condition for the confidence level information obtained by inputting an (N+1)-th frame to the lumen recognition model is satisfied;

FIG. 26 is a flowchart showing an example of a flow of machine learning processing;

FIG. 27 is a flowchart showing an example of a flow of model construction processing;

FIG. 28A is a flowchart showing an example of a flow of medical support processing;

FIG. 28B is a continuation of the flowchart shown in FIG. 28A;

FIG. 29 is a modification example of the flowchart shown in FIGS. 28A and 28B;

FIG. 30 is a conceptual diagram showing an example of processing contents in the controller in a case in which the mark is generated in units of the partition regions without using a vector sum;

FIG. 31 is a conceptual diagram showing an aspect example in which, in a case in which the partition region to which a maximum confidence level among all the confidence levels added to all the partition regions of the map is added is changed, the mark is displayed at a position corresponding to the partition region after the change, and the display of the mark before the change is continued;

FIG. 32 is a conceptual diagram showing a modification example of the example image;

FIG. 33 is a conceptual diagram showing an example of the training data generated in a case in which the lumen is shown in the region other than the central region of the example image shown in FIG. 32;

FIG. 34 is a conceptual diagram showing an example of the training data generated in a case in which the lumen is shown in the central region of the example image shown in FIG. 32;

FIG. 35 is a conceptual diagram showing an example of processing contents in the information processing device in a case in which the lumen recognition model is generated by training a model through machine learning using the training data;

FIG. 36 is a conceptual diagram showing an example of processing contents in the information processing device in a case in which the time-series model is constructed based on a time-series dataset different from the time-series dataset shown in FIG. 12;

FIG. 37 is a conceptual diagram showing a modification example of the processing contents shown in FIG. 17;

FIG. 38 is a conceptual diagram showing a modification example of the processing contents shown in FIG. 20;

FIG. 39 is a conceptual diagram showing a modification example of the processing contents shown in FIG. 23;

FIG. 40 is a conceptual diagram showing a modification example of the processing contents shown in FIG. 25;

FIG. 41 is a conceptual diagram showing a form example in which the mark is displayed along an outer edge of the frame displayed on the screen;

FIG. 42 is a conceptual diagram showing a form example in which the mark is displayed in a superimposed manner on the frame by displaying at least one marker corresponding to the position of the lumen existence region among a plurality of hidden markers associated with the map and/or the frame;

FIG. 43 is a conceptual diagram showing a form example in which an outer contour line that is a line that outlines an outer contour of the lumen existence region is displayed in a superimposed manner on the frame; and

FIG. 44 is a conceptual diagram showing an example of a series of processing in which a processor included in a computer issues a processing execution request to an external device via a network, the external device executes processing in response to the processing execution request, and the processor included in the computer receives a processing result from the external device.

### DETAILED DESCRIPTION

Hereinafter, examples of embodiments of a medical support device, an endoscope system, a medical support method, and a program according to the present disclosure will be described with reference to the accompanying drawings. It should be noted that the present disclosure is also applicable to a program and a computer program product.

First, the terms used in the following description will be described.

CPU is an abbreviation for “central processing unit”. GPU is an abbreviation for “graphics processing unit”. GPGPU is an abbreviation for “general-purpose computing on graphics processing units”. APU is an abbreviation for “accelerated processing unit”. TPU is an abbreviation for “tensor processing unit”. RAM is an abbreviation for “random-access memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. WLI is an abbreviation for “white light imaging”. BLI is an abbreviation for “blue light imaging”. LCI is an abbreviation for “linked color imaging”. NBI is an abbreviation for “narrow-band imaging”. CT is an abbreviation for “computed tomography”. MRI is an abbreviation for “magnetic resonance imaging”. I/F is an abbreviation for “interface”. LAN is an abbreviation for “local area network”. WAN is an abbreviation for “wide area network”. 5G is an abbreviation for “5th generation mobile communication system”.

Hereinafter, a processor with a reference numeral (hereinafter, simply referred to as a “processor”) may be one computing device or may be a combination of a plurality of computing devices. Furthermore, the processor may be one type of computing device or may be a combination of a plurality of types of computing devices. Examples of the computing device include a CPU, a GPU, a GPGPU, an APU, and a TPU.

Hereinafter, a memory with a reference numeral is a memory such as a RAM that temporarily stores information, and is used by the processor as a working memory.

Hereinafter, a storage with a reference numeral is one or a plurality of non-volatile storage devices that store various programs, various parameters, and the like. Examples of the non-volatile storage device include a flash memory, a magnetic disk, and a magnetic tape. Another example of the storage is a cloud storage.

In the following embodiment, an external I/F with a reference numeral controls the transmission and reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi (registered trademark), and Bluetooth (registered trademark).

In the following embodiment, “A and/or B” has the same meaning as “at least one of A or B”. That is, “A and/or B” may mean only A, may mean only B, or may mean a combination of A and B. In the present specification, the same concept as “A and/or B” is applied to a case in which the connection of three or more matters is expressed by “and/or”.

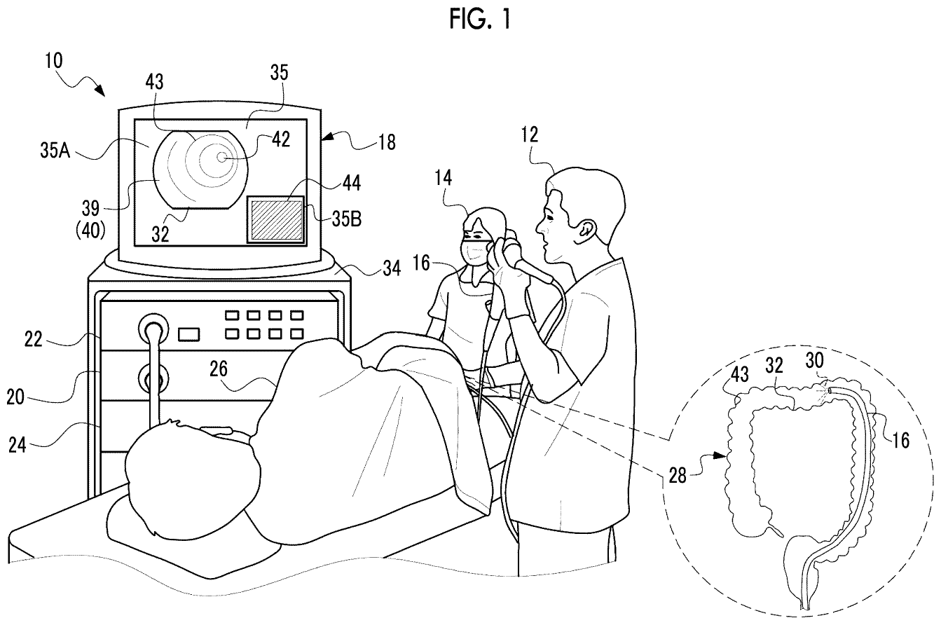

FIG. 1 is a conceptual diagram showing an example of an aspect in which an endoscope system 10 is used. As shown in FIG. 1, the endoscope system 10 is used by a doctor 12 in an endoscopy and the like. A staff member 14 such as a nurse assists with the endoscopy.

The endoscope system 10 is communicably connected to a communication device (not shown), and information obtained by the endoscope system 10 is transmitted to the communication device. Examples of the communication device include a server, a personal computer, and/or a tablet terminal that manage various types of information, such as electronic medical records. The communication device receives the information transmitted from the endoscope system 10, and executes processing using the received information (for example, processing of storing the information in the electronic medical record or the like).

The endoscope system 10 comprises an endoscope 16, a display device 18, a light source device 20, a control device 22, and a medical support device 24. In the present embodiment, the endoscope system 10 is an example of an “endoscope system” according to the present disclosure, the endoscope 16 is an example of an “endoscope” according to the present disclosure, and the medical support device 24 is an example of a “medical support device” according to the present disclosure.

The endoscope system 10 is a modality for performing a medical examination on a large intestine 28, which is a luminal organ included in a body of a subject 26 (for example, a patient), by using the endoscope 16. The large intestine 28 in the present embodiment is an object to be observed by the doctor 12.

The endoscope 16 is used by the doctor 12, and is inserted into the body of the subject 26. In the present embodiment, the endoscope 16 is inserted into the large intestine 28 of the subject 26. The large intestine 28 in the present embodiment is an example of a “luminal organ” according to the present disclosure.

The endoscope system 10 images an inside of the large intestine 28 including a lumen 42 by using the endoscope 16 inserted into the large intestine 28 of the subject 26, and performs various medical treatments on the large intestine 28 as necessary.

The large intestine 28 has the lumen 42. The endoscope 16 is inserted into the lumen 42. A position of the lumen 42 in the large intestine 28 can be medically specified based on a form pattern of a plurality of folds 43 (for example, a shape, an orientation, and the like of the plurality of folds 43) which are characteristic regions in the large intestine 28. In the present embodiment, as will be described in detail later, the position of the lumen 42 is recognized by AI that has been trained using various types of information, such as the form pattern of the plurality of folds 43, through machine learning, and a result of the recognition is provided as visually ascertainable information to the doctor 12. The lumen 42 in the present embodiment is an example of a “lumen” according to the present disclosure.

The endoscope system 10 acquires an image showing an aspect including the lumen 42 in the large intestine 28 by imaging the inside of the large intestine 28 including the lumen 42, and outputs the acquired image. In the present embodiment, the endoscope system 10 has an optical imaging function of emitting light 30 in the large intestine 28 and imaging reflected light obtained by being reflected by an intestinal wall 32 of the large intestine 28.

Here, the endoscopy of the large intestine 28 has been described as an example, but this is merely an example, and the present disclosure is applicable to an endoscopy of a luminal organ, such as an esophagus, a stomach, a duodenum, or a trachea.

The light source device 20, the control device 22, and the medical support device 24 are installed in a wagon 34. The wagon 34 is provided with a plurality of tables along an up-down direction, and the medical support device 24, the light source device 20, and the control device 22 are installed from a lower table to an upper table. Furthermore, the display device 18 is installed on an uppermost table in the wagon 34.

The control device 22 controls the entire endoscope system 10. The control device 22 executes various types of processing on the image obtained by imaging the intestinal wall 32 with the endoscope 16. In addition, the medical support device 24 executes AI-based processing or the like on the image that has been subjected to various types of processing by the control device 22, under the control of the control device 22, and outputs various types of information including a processing result of the AI-based processing or the like. Examples of an output destination of the various types of information include the display device 18, a stationary storage medium (for example, a storage mounted in the endoscope system 10, a storage of a server or the like that is connected to the endoscope system 10 in a communicable manner, and the like), and/or a portable storage medium (for example, a memory card, a USB flash drive, and the like).

The display device 18 displays various types of information (for example, various types of information output from the medical support device 24). Examples of the display device 18 include a liquid-crystal display and an EL display. A tablet terminal equipped with a display may be used instead of the display device 18 or together with the display device 18.

The display device 18 displays a screen 35. A plurality of display regions are included on the screen 35. The plurality of display regions are arranged on the screen 35. In the example shown in FIG. 1, a first display region 35A and a second display region 35B are shown as examples of the plurality of display regions. The first display region 35A has a larger size than the second display region 35B. The first display region 35A is used as a main display region, and the second display region 35B is used as a sub-display region. A size relationship between the first display region 35A and the second display region 35B is not limited to this, and need only be a size relationship that can be included within the screen 35.

An endoscopic video image 39 is displayed in the first display region 35A. The endoscopic video image 39 is obtained by executing various types of processing on a plurality of images arranged in time series obtained by imaging the inside of the large intestine 28 of the subject 26 with the endoscope 16. The intestinal wall 32 shown in the endoscopic video image 39 includes the lumen 42 as a region of interest (that is, an observation target region) at which the doctor 12 gazes, and the doctor 12 can visually recognize the aspect of the intestinal wall 32 including the lumen 42 through the endoscopic video image 39.

The image displayed in the first display region 35A is one frame 40 included in a video image including a plurality of frames 40 arranged in time series. That is, the plurality of frames 40 arranged in time series are displayed in the first display region 35A at predetermined frame rates (for example, a dozen frames/second or a few dozen frames/second). The frame 40 in the present embodiment is an example of a “medical image” according to the present disclosure.

Examples of the video image displayed in the first display region 35A include a video image in a live view mode. The live view mode is merely an example, and the video image may be a video image, such as a video image in a post view mode, that is temporarily stored in a memory or the like and then displayed. In addition, each frame included in a video image for recording stored in the memory or the like may be reproduced and displayed as the endoscopic video image 39 on the screen 35 (for example, in the first display region 35A).

The second display region 35B is displayed at the lower right of the screen 35 in a front view. The second display region 35B may be displayed at any position as long as the position is within the screen 35 of the display device 18, but is preferably displayed at a position that is comparable with the endoscopic video image 39. Auxiliary information 44 for assisting the doctor 12 in a medical determination or the like is displayed in the second display region 35B. The auxiliary information 44 is information to be referred to by the doctor 12. Examples of the auxiliary information 44 include various types of information on the subject 26 in which the endoscope 16 is inserted and/or various types of information obtained by executing medical support processing which will be described later.

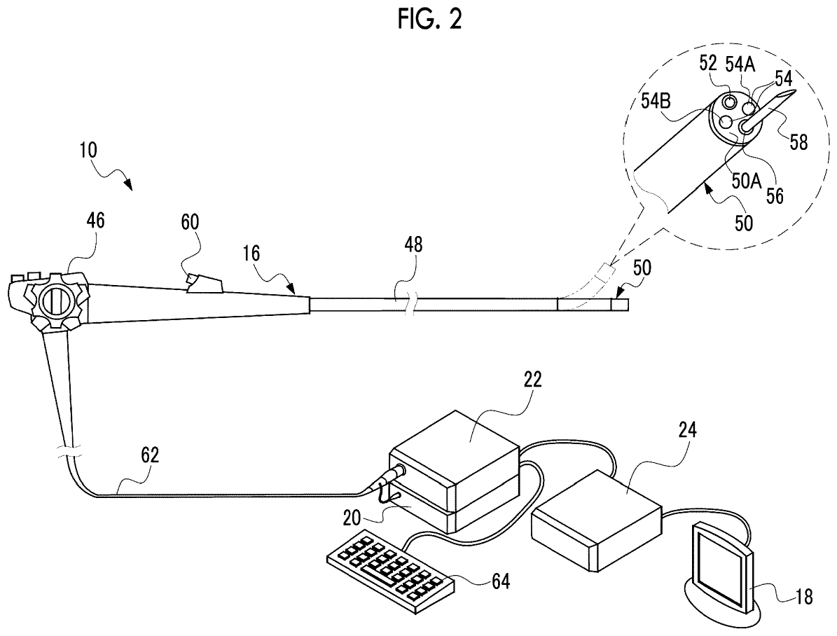

FIG. 2 is a conceptual diagram showing an example of an overall configuration of the endoscope system 10. As shown in FIG. 2, the endoscope 16 comprises an operating part 46 and an insertion part 48. The insertion part 48 is partially curved by the operation of the operating part 46. The insertion part 48 is inserted into the large intestine 28 while being curved along the shape of the large intestine 28 (see FIG. 1) in accordance with the operation of the operating part 46 performed by the doctor 12 (see FIG. 1).

A camera 52, an illumination device 54, and a treatment tool opening 56 are provided at a distal end portion 50 of the insertion part 48. A portion of the camera 52 (for example, an imaging optical system) and a portion of the illumination device 54 (for example, an irradiation optical system) are exposed from a distal end surface 50A of the distal end portion 50.

The camera 52 is mounted in the endoscope 16 and is inserted into a body cavity (here, as an example, the lumen 42) of the subject 26 to image the observation target region. Examples of the camera 52 include a CMOS camera. However, this is merely an example, and the camera 52 may be other types of cameras, such as CCD cameras. In the present embodiment, the camera 52 generates an image showing the aspect including the lumen 42 in the large intestine 28 by imaging the inside of the large intestine 28 including the lumen 42. The image generated by the camera 52 is an image of which an outer shape is circular. For example, the image generated by the camera 52 is processed into a shape in which an upper end portion and a lower end portion are masked, by the control device 22. Accordingly, as shown in FIG. 1, an image in which an upper end edge and a lower end edge are linear and a left side edge and a right side edge are arc-shaped is generated as the frame 40.

The illumination device 54 includes illumination windows 54A and 54B. The illumination windows 54A and 54B are provided on the distal end surface 50A. The illumination device 54 emits the light 30 (see FIG. 1) through the illumination windows 54A and 54B. Examples of the type of the light 30 emitted from the illumination device 54 include light for WLI (for example, white light), light for LCI (for example, light obtained by combining red light, green light, and blue light), light for BLI (for example, blue light), and/or light for NBI (for example, light obtained by combining blue light and green light). The camera 52 images the inside of the large intestine 28 using an optical method in a state in which the inside of the large intestine 28 is irradiated with the light 30 (see FIG. 1) by the illumination device 54.

The treatment tool opening 56 is an opening for allowing a treatment tool 58 to protrude from the distal end portion 50. Furthermore, the treatment tool opening 56 is also used as a suction port for suctioning blood, internal contaminants, and the like and a sending-out port for sending out fluid.

A treatment tool insertion port 60 is formed at the operating part 46, and the treatment tool 58 is inserted into the insertion part 48 through the treatment tool insertion port 60. The treatment tool 58 passes through the insertion part 48 to protrude from the treatment tool opening 56 to the outside. In the example shown in FIG. 2, an aspect is shown in which a biopsy needle protrudes through the treatment tool opening 56 as the treatment tool 58. Here, the biopsy needle has been described as an example of the treatment tool 58, but this is merely an example, and the treatment tool 58 may be grasping forceps, a papillotomy knife, a snare, a catheter, a guide wire, a cannula, and/or a biopsy needle with a guide sheath.

The endoscope 16 is connected to the light source device 20 and the control device 22 through a universal cord 62. The medical support device 24 and a reception device 64 are connected to the control device 22. Furthermore, the display device 18 is connected to the medical support device 24. That is, the control device 22 is connected to the display device 18 via the medical support device 24.

Here, since the medical support device 24 is used as an example of an external device for expanding the functions of the control device 22, the form example has been described in which the control device 22 and the display device 18 are indirectly connected to each other via the medical support device 24, but this is merely an example. For example, the display device 18 may be directly connected to the control device 22. In this case, for example, the functions of the medical support device 24 need only be carried out in the control device 22, or the control device 22 need only have a function of directing a server (not shown) to execute the same processing as the processing (for example, the medical support processing which will be described later) executed by the medical support device 24, receiving a processing result obtained by the server, and using the processing result.

The reception device 64 receives an instruction from the doctor 12, and outputs the received instruction as an electric signal to the control device 22. Examples of the reception device 64 include a keyboard, a mouse, a touch panel, a foot switch, a microphone, and/or a

remote control device.

The control device 22 controls the light source device 20, transmits and receives various signals to and from the camera 52, or transmits and receives various signals to and from the medical support device 24.

The light source device 20 emits light to supply the light 30 to the illumination device 54 under the control of the control device 22. A light guide is provided in the illumination device 54, and the light 30 supplied from the light source device 20 is emitted from the illumination windows 54A and 54B through the light guide. The control device 22 causes the camera 52 to perform the imaging in a state in which the light 30 is emitted from the illumination windows 54A and 54B. The control device 22 generates the plurality of frames 40 arranged in time series by processing the outer shape of the image obtained by imaging performed by the camera 52 or adjusting an image quality or the like of the image. The control device 22 outputs the endoscopic video image 39 including the plurality of generated frames 40 arranged in time series to a predetermined output destination (for example, the medical support device 24).

The medical support device 24 executes various types of processing on the endoscopic video image 39 input from the control device 22 to support a medical treatment (here, for example, endoscopy). The medical support device 24 outputs the endoscopic video image 39, which has been subjected to various types of processing, to a predetermined output destination (for example, the display device 18).

Here, the form example has been described in which the endoscopic video image 39 output from the control device 22 is output to the display device 18 via the medical support device 24, but this is merely an example. For example, an aspect may be adopted in which the control device 22 and the display device 18 are connected to each other, and the endoscopic video image 39, which has been subjected to various types of processing by the medical support device 24, is displayed on the display device 18 via the control device 22.

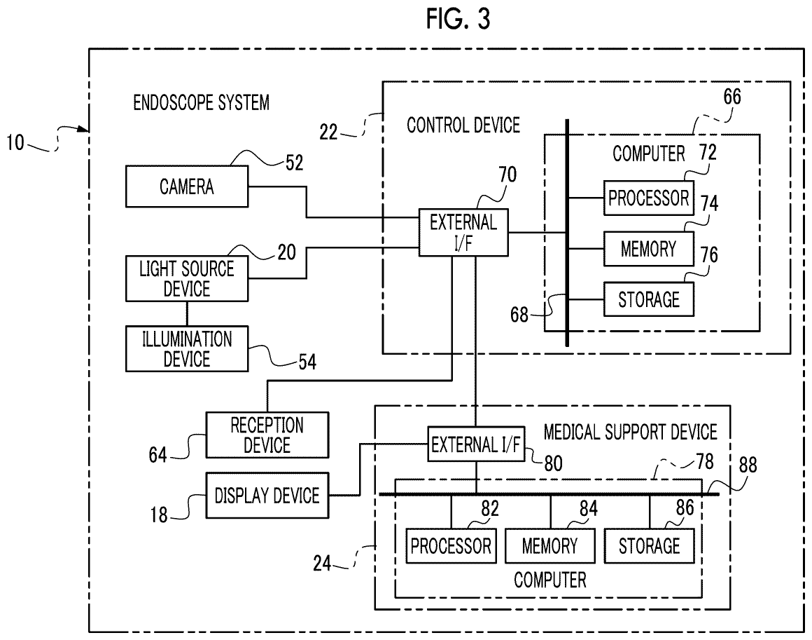

FIG. 3 is a block diagram showing an example of a hardware configuration of an electrical system of the endoscope system 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) existing outside the

control device 22 and the processor 72.

The camera 52 is connected to the external I/F 70 as one of the first external devices, and the external I/F 70 transmits and receives various types of information between the camera 52 and the processor 72. The processor 72 controls the camera 52 through the external I/F 70. In addition, the processor 72 acquires an image generated by imaging the inside of the large intestine 28 (see FIG. 1) with the camera 52 via the external I/F 70, and performs various types of processing on the acquired image to generate the endoscopic video image 39 (see FIG. 1).

The light source device 20 is connected to the external I/F 70 as one of the first external devices, and the external I/F 70 transmits and receives various types of information between the light source device 20 and the processor 72. The light source device 20 supplies the light 30 to the illumination device 54 under the control of the processor 72. The illumination device 54 emits the light 30 supplied from the light source device 20.

The reception device 64 is connected to the external I/F 70 as one of the first external devices, and the processor 72 acquires the instruction received by the reception device 64 via the external I/F 70 and executes the processing corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the computer 78 is an example of a “computer” according to the present disclosure, and the processor 82 is an example of a “processor” according to the present disclosure.

It should be noted that a hardware configuration (that is, the processor 82, the memory 84, and the storage 86) of the computer 78 is essentially the same as the hardware configuration of the computer 66, and thus the description of the hardware configuration of the computer 78 will be omitted here.

The external I/F 80 transmits and receives various types of information between one or more devices (hereinafter, also referred to as “second external devices”) existing outside the medical support device 24 and the processor 82.

The control device 22 is connected to the external I/F 80 as one of the second external devices. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic video image 39 (see FIG. 1) from the processor 72 of the control device 22 via the external I/Fs 70 and 80, and executes various types of processing on the acquired endoscopic video image 39. The various types of processing executed by the processor 82 include AI-based processing (for example, processing using a lumen recognition model 92 described later).

The display device 18 is connected to the external I/F 80 as one of the second external devices. The processor 82 controls the display device 18 via the external I/F 80 such that various types of information (for example, the endoscopic video image 39 that has been subjected to various types of processing) are displayed on the display device 18.

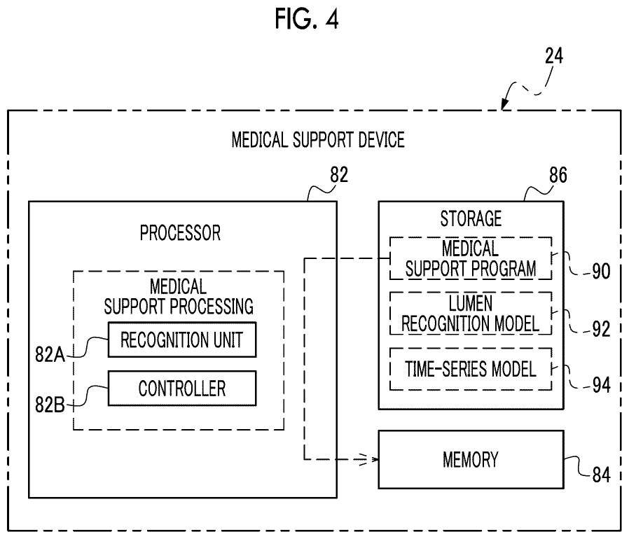

FIG. 4 is a block diagram showing an example of main functions of the processor 82 included in the medical support device 24 and an example of the information stored in the storage 86. As shown in FIG. 4, a medical support program 90 is stored in the storage 86. The medical support program 90 in the present embodiment is an example of a “program” according to the present disclosure.

The processor 82 reads out the medical support program 90 from the storage 86, and executes the readout medical support program 90 on the memory 84 to perform medical support processing. The medical support processing is carried out by the processor 82 operating as a recognition unit 82A and a controller 82B in accordance with the medical support program 90 executed on the memory 84.

The lumen recognition model 92 and a time-series model 94 are stored in the storage 86. As will be described in detail later, the lumen recognition model 92 is a machine learning model, and is used by the recognition unit 82A. Examples of the machine learning model include a neural network. The time-series model 94 is a probabilistic model, and is used by the recognition unit 82A. Examples of the probabilistic model include a Markov model.

A first example of the Markov model is a Markov model based on a Markov chain. The Markov chain is a probabilistic model that has a Markov property in which a current state affects a next state and that predicts the next state based on a probability of state transition. Examples of the Markov chain include a simple Markov chain and an N-th order Markov chain. Examples of the simple Markov chain include a discrete-time Markov chain and a continuous-time Markov chain. A second example of the Markov model is a Markov model based on a hidden Markov model.

The Markov model is merely an example, and a time-series model including a deep learning model (for example, a deep learning model based on a convolutional neural network, a recurrent neural network, a transformer, or the like) or a time-series analysis mechanism (for example, a time-series unit based on an autoregressive model, a moving average model, an autoregressive moving average model, an autoregressive integrated moving average model, an autoregressive conditional variance model, or the like) may be applied instead of the Markov model.

In addition, the time-series model 94 may be a time-series model including two or more of the Markov model, the deep learning model, or the time-series analysis mechanism.

In the present embodiment, the lumen recognition model 92 is an example of a “trained model” according to the present disclosure, and the time-series model 94 is an example of a “time-series model” according to the present disclosure.

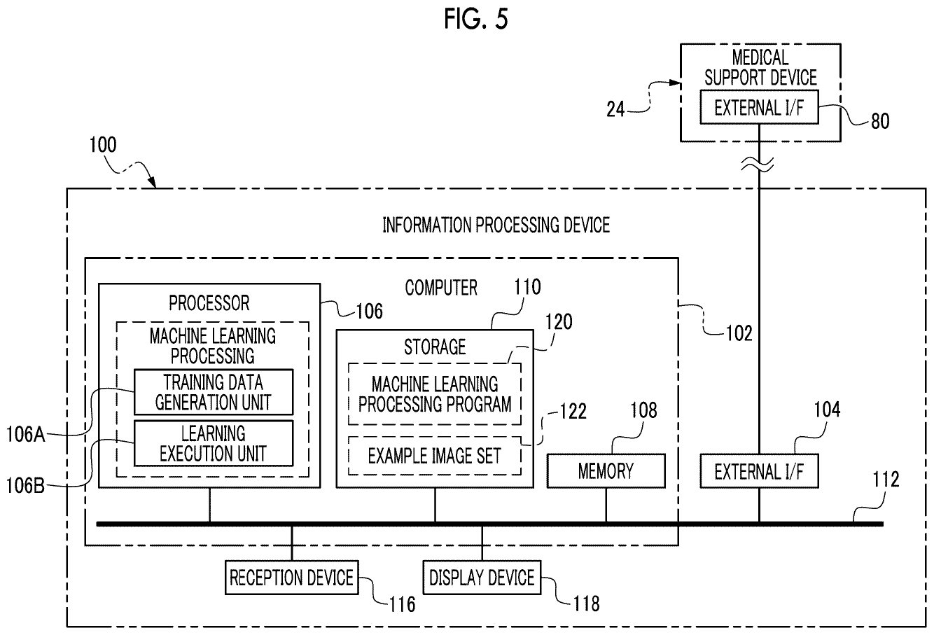

FIG. 5 is a block diagram showing an example of a hardware configuration of an electrical system of an information processing device 100 used to generate the lumen recognition model 92 and the time-series model 94. As shown in FIG. 5, the information processing device 100 comprises a computer 102 and an external I/F 104. The computer 102 comprises a processor 106, a memory 108, and a storage 110. The processor 106, the memory 108, the storage 110, and the external I/F 104 are connected to a bus 112.

It should be noted that a hardware configuration (that is, the processor 106, the memory 108, and the storage 110) of the computer 102 is essentially the same as the hardware configuration of the computer 66, and thus the description of the hardware configuration of the computer 102 will be omitted here.

The information processing device 100 comprises a reception device 116. The reception device 116 is, for example, a keyboard and/or a mouse, and receives an instruction from a user of the information processing device 100 and the like. The reception device 116 is connected to the bus 112. The processor 106 acquires the instruction received by the reception device 116 and operates in accordance with the acquired instruction.

A display device 118 displays various types of information including the image. Examples of the display device 118 include a liquid-crystal display and an EL display. The display device 118 is connected to the bus 112. The processor 106 displays the results obtained by executing various types of processing on the display device 118.

The external I/F 104 transmits and receives various types of information between one or more devices (hereinafter, also referred to as “third external devices”) existing outside the information processing device 100 and the processor 106. The medical support device 24 is connected to the external I/F 104 as one of the third external devices. In the example shown in FIG. 5, the external I/F 80 of the medical support device 24 is connected to the external I/F 104. The external I/F 104 controls the transmission and reception of various types of information between the processor 82 (see FIGS. 3 and 4) of the medical support device 24 and the processor 106 of the information processing device 100. For example, the information processing device 100 generates the lumen recognition model 92 and the time-series model 94, and transmits the generated lumen recognition model 92 and time-series model 94 to the medical support device 24 via the external I/Fs 80 and 104 in response to a request from the medical support device 24.

A machine learning processing program 120 is stored in the storage 110. The processor 106 reads out the machine learning processing program 120 from the storage 110, and executes the readout machine learning processing program 120 on the memory 108 to perform machine learning processing. The machine learning processing is carried out by the processor 106 operating as a training data generation unit 106A and a learning execution unit 106B in accordance with the machine learning processing program 120 executed on the memory 108.

An example image set 122 is stored in the storage 110. As will be described in detail later, the example image set 122 is used by the training data generation unit 106A.

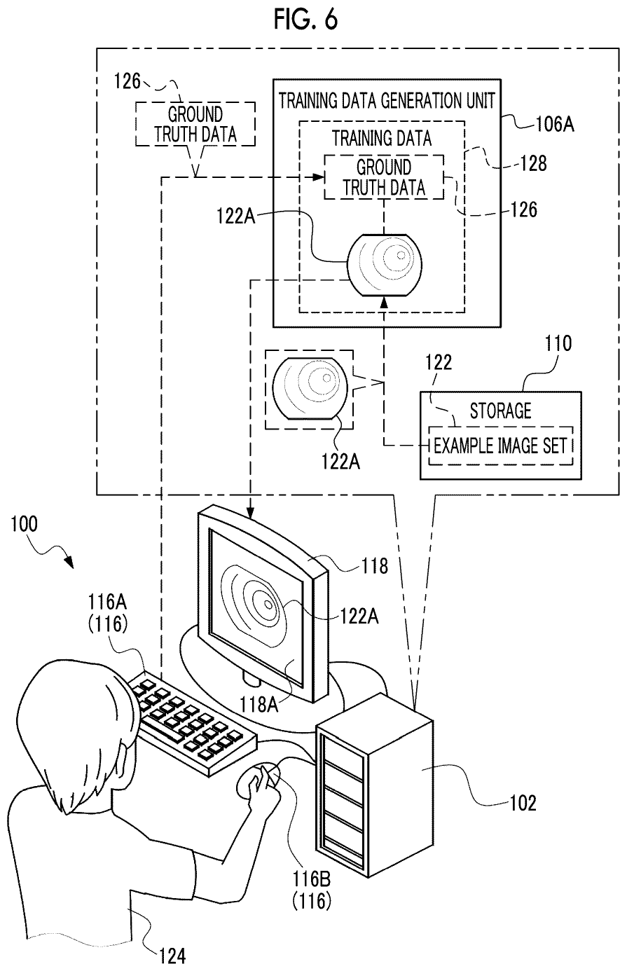

FIG. 6 is a conceptual diagram showing an example of processing contents in the training data generation unit 106A. As shown in FIG. 6, the information processing device 100 is used by an annotator 124. The annotator 124 means an operator who adds annotations for machine learning to given data (that is, an operator who performs labeling).

In the example shown in FIG. 6, a keyboard 116A and a mouse 116B are shown as examples of the reception device 116. The annotator 124 issues an instruction to the computer 102 via the keyboard 116A and the mouse 116B.

The example image set 122 includes a plurality of example images 122A showing different contents. The example image 122A is an image determined in advance as a medical image to be used for object recognition processing (for example, processing in which the recognition unit 82A recognizes the lumen 42 based on the frame 40 and the lumen recognition model 92). The image determined in advance as the medical image to be used for the object recognition processing is an image corresponding to the frame 40. In other words, the image corresponding to the frame 40 can also be referred to as an image that represents the frame 40. In other words, the image that represents the frame 40 can also be referred to as an image showing a sample of the frame 40. Here, a first example of the image showing the sample of the frame 40 is an image obtained by actually imaging the inside of the large intestine with the camera. A second example of the image showing the sample of the frame 40 is a virtually created image (for example, an image generated by generative AI, such as Stable Diffusion or Midjourney).

The training data generation unit 106A acquires the example image 122A from the example image set 122 in response to the instruction received by the reception device 116. The training data generation unit 106A displays the example image 122A on a screen 118A of the display device 118. In a state in which the example image 122A is displayed on the screen 118A, the annotator 124 indicates a lumen correspondence position, which is the position of the lumen shown in the example image 122A, in the example image 122A, with respect to the training data generation unit 106A via the reception device 116. The training data generation unit 106A associates ground truth data 126 with the example image 122A based on the lumen correspondence position indicated via the reception device 116, to generate training data 128. The association of the ground truth data 126 with the example image 122A is carried out by adding an annotation capable of specifying the lumen correspondence position as the ground truth data 126 to the lumen correspondence position in the example image 122A.

In this way, the training data generation unit 106A repeatedly executes the processing of associating the ground truth data 126 with each of the example images 122A included in the example image set 122 in response to the instruction issued from the annotator 124, to generate a plurality of pieces of training data 128.

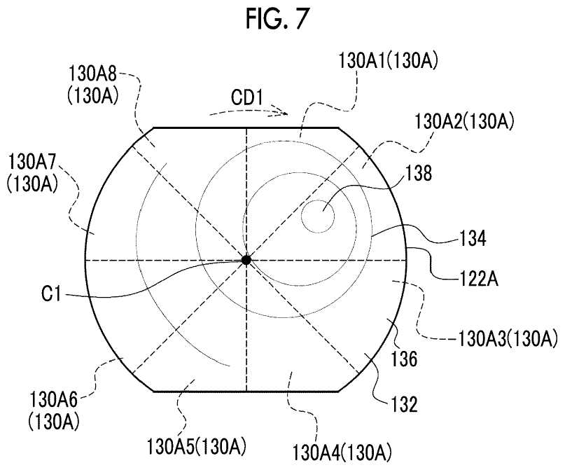

FIG. 7 is a conceptual diagram showing an example of a composition of the example image 122A. As shown in FIG. 7, a large intestine 132 is shown in the example image 122A. In the example shown in FIG. 7, an intestinal wall 136 in which a plurality of folds 134 are formed and a lumen 138 are shown in the example image 122A.

The example image 122A is partitioned into a plurality of partition regions 130A. Eight partition regions 130A1 to 130A8 are included in the plurality of partition regions 130A. The partition regions 130A1 to 130A8 are regions that radially exist from a center C1 of the example image 122A toward an outer edge of the example image 122A, and are disposed along a circumferential direction CD1 (in other words, around the center C1) of the example image 122A.

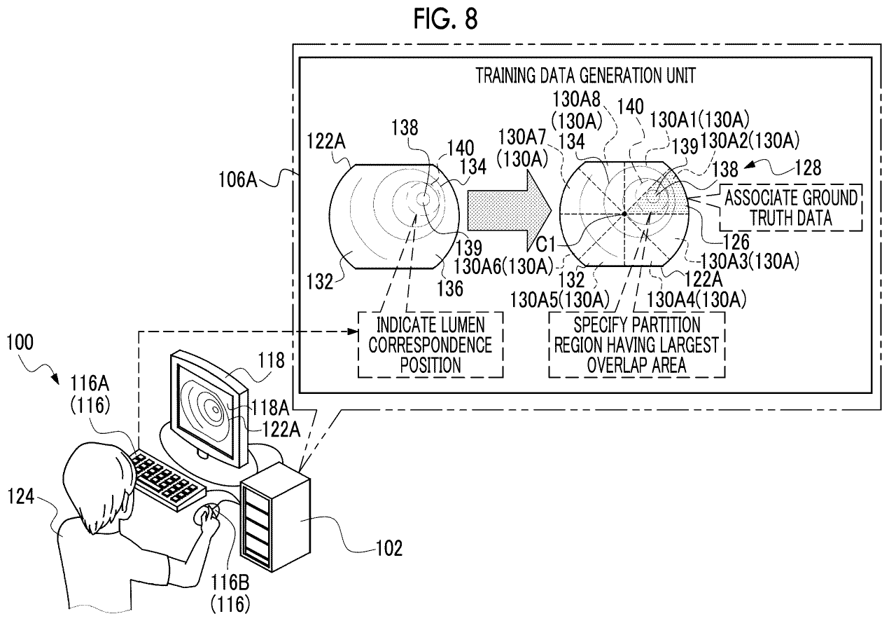

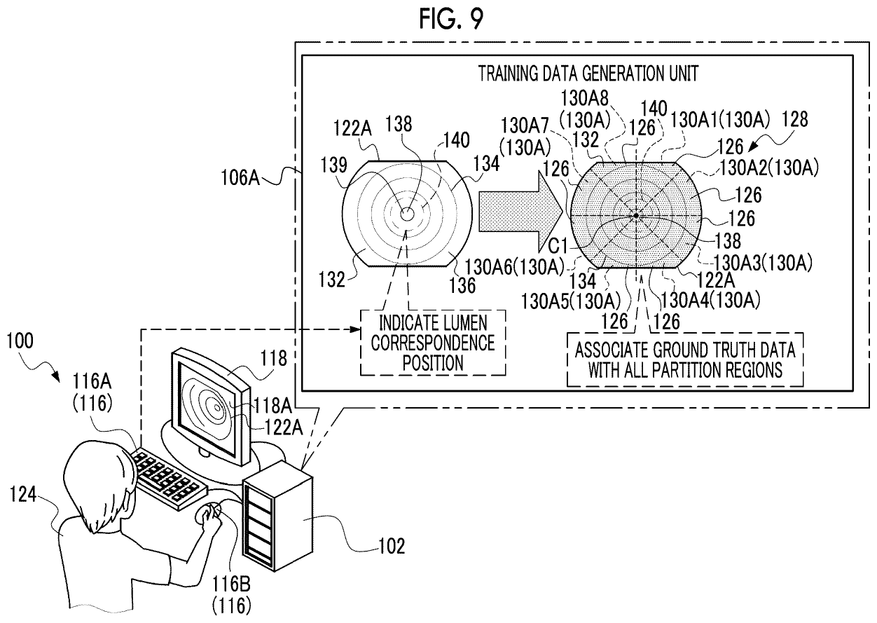

FIGS. 8 and 9 are conceptual diagrams showing an example of a method in which the training data generation unit 106A associates the ground truth data 126 with the example image 122A to generate the training data 128.

As shown in FIG. 8, in a state in which the example image 122A is displayed on the screen 118A, the annotator 124 indicates a lumen correspondence position 139, which is the position of the lumen 138 shown in the example image 122A, in the example image 122A, with respect to the training data generation unit 106A via the reception device 116. The training data generation unit 106A displays a circular frame 140 in a superimposed manner on the example image 122A in response to the instruction received by the reception device 116, and disposes the frame 140 at a position surrounding the lumen 138 shown in the example image 122A. The frame 140 is a mark that defines the lumen correspondence position 139 in the example image 122A. That is, a position of a region surrounded by the frame 140 in the example image 122A is the lumen correspondence position 139. The size and the position of the frame 140 are freely changed on the screen 118A in response to the instruction received by the reception device 116. Here, the shape of the frame 140 is a circular shape, but may be a shape other than circular. The size of the frame 140 can be changed in response to the instruction received by the reception device 116.

The annotator 124 issues a confirmation instruction, which is an instruction to confirm the lumen correspondence position 139, to the training data generation unit 106A via the reception device 116 in a state in which the frame 140 is disposed at the position surrounding the lumen 138. As a result, the training data generation unit 106A confirms the lumen correspondence position 139.

The training data generation unit 106A specifies the partition region 130A having a largest overlap area with the frame 140 that defines the lumen correspondence position 139 among the plurality of partition regions 130A. Then, the training data generation unit 106A generates the training data 128 by associating the ground truth data 126 with the specified partition region 130A (in the example shown in FIG. 8, the partition region 130A2) as the annotation capable of specifying the partition region 130A in which the lumen 138 is shown.

FIG. 8 shows an example of a method for generating the training data 128 in a case in which the lumen 138 is shown in a region other than the central region in the example image 122A, whereas FIG. 9 shows an example of a method for generating the training data 128 in a case in which the lumen 138 is shown in the central region in the example image 122A. As shown in FIG. 9, in a case in which the lumen 138 is shown in the central region in the example image 122A, the training data generation unit 106A generates the training data 128 by associating the ground truth data 126 with each of all the partition regions 130A (that is, the partition regions 130A1 to 130A8).

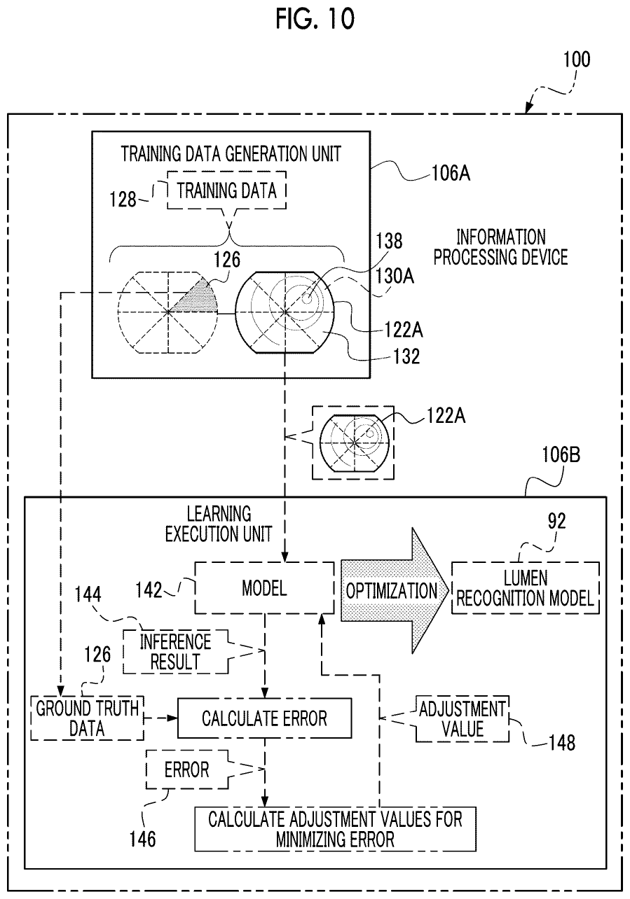

FIG. 10 is a conceptual diagram showing an example of an aspect in which the learning execution unit 106B executes machine learning using the training data 128 to generate the lumen recognition model 92. As shown in FIG. 10, in the information processing device 100, the learning execution unit 106B acquires the training data 128 generated by the training data generation unit 106A. The learning execution unit 106B executes the machine learning using the training data 128.

In the example shown in FIG. 10, the learning execution unit 106B includes a model 142. Examples of the model 142 include a neural network. Examples of the neural network include a convolutional neural network. The learning execution unit 106B inputs the example image 122A included in the training data 128 to the model 142. In a case in which the example image 122A is input, the model 142 performs an inference to output an inference result 144. The learning execution unit 106B calculates an error 146 between the inference result 144 and the ground truth data 126 included in the training data 128.

The learning execution unit 106B calculates a plurality of adjustment values 148 for minimizing the error 146. Then, the learning execution unit 106B adjusts a plurality of optimization variables in the model 142 by using the plurality of adjustment values 148, to optimize the model 142. For example, the plurality of optimization variables mean a plurality of coupling weights and a plurality of offset values included in the model 142.

The learning execution unit 106B repeatedly executes learning processing of inputting the example image 122A to the model 142, calculating the error 146, calculating the plurality of adjustment values 148, and adjusting the plurality of optimization variables in the model 142 using the plurality of pieces of training data 128. That is, the learning execution unit 106B adjusts the plurality of optimization variables in the model 142 using the plurality of adjustment values 148 calculated such that the error 146 is minimized for each of the plurality of example images 122A included in the plurality of pieces of training data 128, to optimize the model 142. The lumen recognition model 92 is generated by optimizing the model 142 in this manner. The lumen recognition model 92 is transmitted from the information processing device 100 to the medical support device 24 via the external I/Fs 80 and 104 (see FIG. 5), and is received by the medical support device 24. Then, in the medical support device 24, the lumen recognition model 92 is stored in the storage 86 by the processor 82 (see FIG. 4). The lumen recognition model 92 stored in the storage 86 is used by the recognition unit 82A (see FIG. 4).

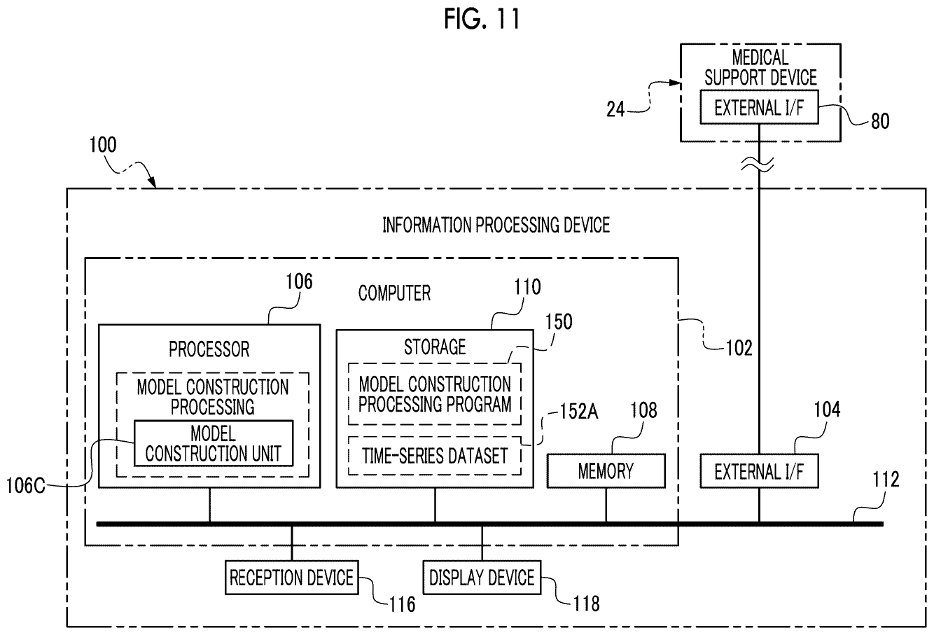

As shown in FIG. 11 as an example, in the information processing device 100, a model construction processing program 150 is stored in the storage 110. The processor 106 reads out the model construction processing program 150 from the storage 110, and executes the readout model construction processing program 150 on the memory 108 to perform model construction processing. The model construction processing is carried out by the processor 106 operating as a model construction unit 106C in accordance with the model construction processing program 150 executed by the processor 106 on the memory 108.

A time-series dataset 152A is stored in the storage 110. As will be described in detail later, the time-series dataset 152A is used by the model construction unit 106C.

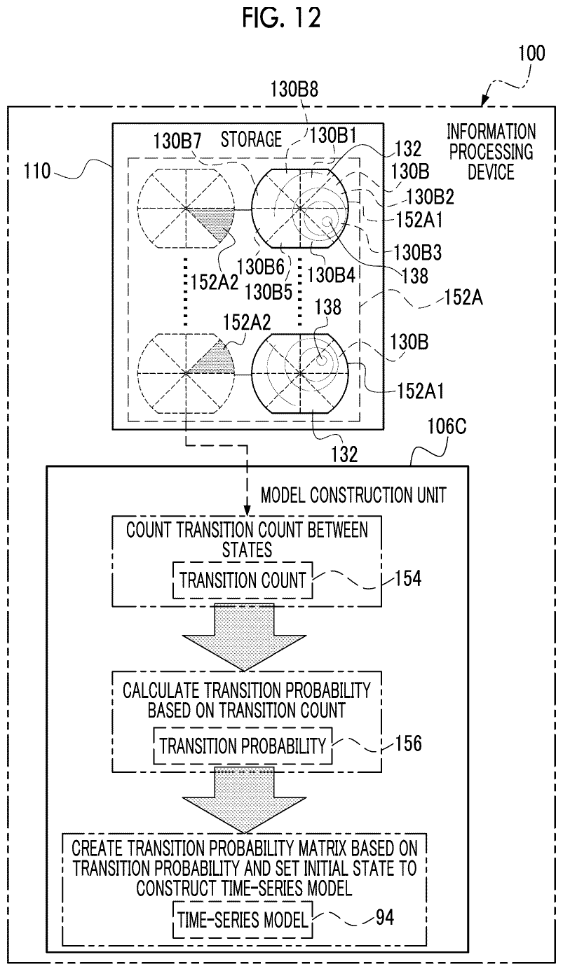

FIG. 12 is a conceptual diagram showing an example of processing contents in which the Markov model based on the Markov chain is constructed as the time-series model 94 by the model construction unit 106C. As shown in FIG. 12, the time-series dataset 152A includes a plurality of time-series images 152A1 arranged in time series. The plurality of time-series images 152A1 are a plurality of images corresponding to the plurality of frames 40 arranged in time series and included in the endoscopic video image 39 (that is, a plurality of images representing the plurality of frames 40 arranged in time series). A first example of the plurality of time-series images 152A1 is a plurality of images obtained in time series by actually imaging the inside of the large intestine with the camera. A second example of the plurality of time-series images 152A1 is a plurality of images (for example, images generated by generative AI such as Stable Diffusion or Midjourney) virtually created as a plurality of images showing samples of the plurality of frames 40 arranged in time series.

Similarly to the example image 122A, the inside of the large intestine 132 is shown in the time-series image 152A1. Further, the time-series image 152A1 is partitioned into a plurality of partition regions 130B by the same method as in the example image 122A. In the example shown in FIG. 12, as the plurality of partition regions 130B, partition regions 130B1 to 130B8 are shown. Geometrical characteristics of the partition regions 130B1 to 130B8 in the time-series image 152A1 correspond to geometrical characteristics of the partition regions 130A1 to 130A8 in the example image 122A. In addition, by the same method as in a case of associating the ground truth data 126 with each example image 122A, ground truth data 152A2 corresponding to the ground truth data 126 is also associated with each time-series image 152A1.

The model construction unit 106C acquires, in time series, a plurality of pieces of ground truth data 152A2 associated with the plurality of time-series images 152A1 arranged in time series, and counts a transition count 154 between the respective states in which the lumen 138 is shown in the partition region 130A in the plurality of time-series images 152A1 arranged in time series from the plurality of pieces of ground truth data 152A2 arranged in time series. Each state in which the lumen 138 is shown in the partition region 130A is specified from the ground truth data 152A2 associated with the plurality of time-series images 152A1.

Examples of the state in which the lumen 138 is shown in the partition region 130A include eight states, that is, first to eighth states. The first state means a state in which the lumen 138 is shown in the partition region 130A1. The second state means a state in which the lumen 138 is shown in the partition region 130A2. The third state means a state in which the lumen 138 is shown in the partition region 130A3. The fourth state means a state in which the lumen 138 is shown in the partition region 130A4. The fifth state means a state in which the lumen 138 is shown in the partition region 130A5. The sixth state means a state in which the lumen 138 is shown in the partition region 130A6. The seventh state means a state in which the lumen 138 is shown in the partition region 130A7. The eighth state means a state in which the lumen 138 is shown in the partition region 130A8. The transition count 154 means the number of times of transition among the first to eighth states (in other words, the number of times the partition region 130A in which the lumen 138 is shown transitions from one partition region 130A to another partition region 130A among the eight partition regions 130A1 to 130A8).

The model construction unit 106C counts the transition count 154 by pairing a state in an N-th time-series image 152A1 and a state in an (N+1)-th time-series image 152A1, and totals the transition count 154 among the first to eighth states. That is, the model construction unit 106C totals the number of times of transition from a certain state among the first to eighth states to a next state, as the transition count 154.

The model construction unit 106C calculates a transition probability 156 based on the transition count 154. The transition probability 156 means a probability indicating the transition from one state among the first to eighth states to another state among the first to eighth states. For example, in a case in which each of N and M is any natural number of 1 to 8, the transition probability 156 from an N-th state to an M-th state is a value obtained by dividing the transition count 154 from the N-th state to the M-th state by the total transition count from the N-th state.

The model construction unit 106C constructs the time-series model 94 by creating a transition probability matrix based on the transition probability 156 and setting an initial state. In a case in which there are eight states, that is, the first to eighth states, the transition probability matrix created based on the transition probability 156 is an 8×8 matrix. The transition probability matrix is a core of the time-series model 94. The initial state means an initial state (for example, one state selected from the first to eighth states by applying a rule base to the plurality of time-series images 152A1) for starting the prediction performed by the time-series model 94. Here, although eight states, that is, the first to eighth states, are shown, these are merely examples, and there may be fewer than eight states or nine or more states. For example, in a case of seven states, the matrix used as the transition probability matrix is a 7×7 matrix, and in a case of 40 states, the matrix used as the transition probability matrix is a 40×40 matrix.

The time-series model 94 constructed by the model construction unit 106C is transmitted from the information processing device 100 to the medical support device 24 via the external I/Fs 80 and 104 (see FIG. 11), and is received by the medical support device 24. In the medical support device 24, the time-series model 94 is stored in the storage 86 by the processor 82 (see FIG. 4). The time-series model 94, which is stored in the storage 86, is used by the recognition unit 82A (see FIG. 4).

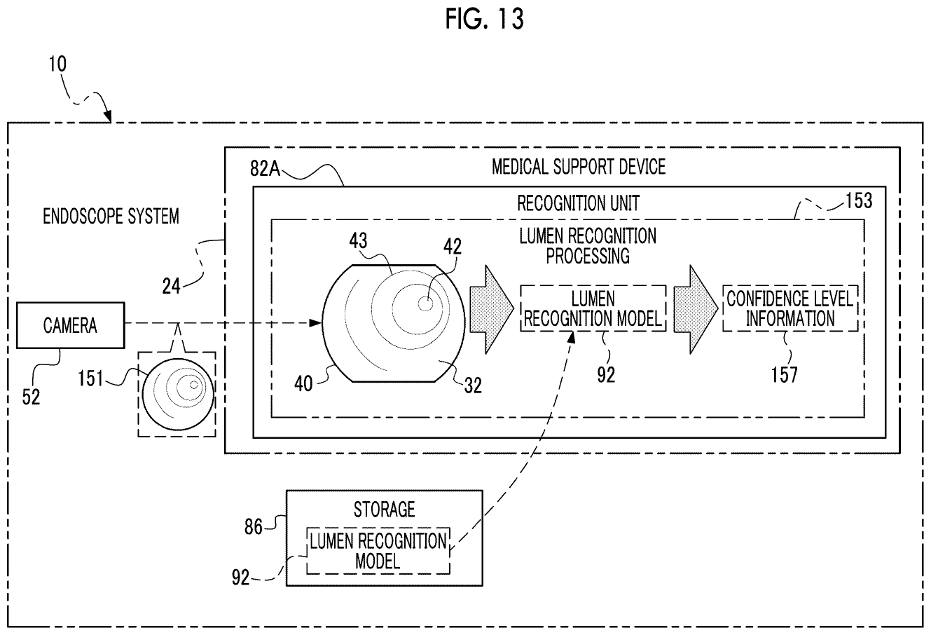

FIG. 13 shows an example of processing contents in the recognition unit 82A. As shown in FIG. 13, an image 151 obtained by imaging the intestinal wall 32 in the large intestine 28 including the lumen 42 with the camera 52 is acquired by the recognition unit 82A. The recognition unit 82A generates the frame 40 by executing various types of processing on the image 151. In the example shown in FIG. 13, the intestinal wall 32 having the folds 43 and the lumen 42 are shown in the frame 40.

The recognition unit 82A executes lumen recognition processing 153 on the frame 40. The lumen recognition processing 153 is processing of recognizing the lumen 42 shown in the frame 40 using the lumen recognition model 92 stored in the storage 86 (in other words, processing of specifying the existence position of the lumen 42, which is shown in the frame 40, in the frame 40 using the lumen recognition model 92). The recognition unit 82A acquires the frame 40 from the camera 52, and inputs the acquired frame 40 to the lumen recognition model 92 to cause the lumen recognition model 92 to generate confidence level information 157. The confidence level information 157 in the present embodiment is an example of “information obtained from the trained model” according to the present disclosure.

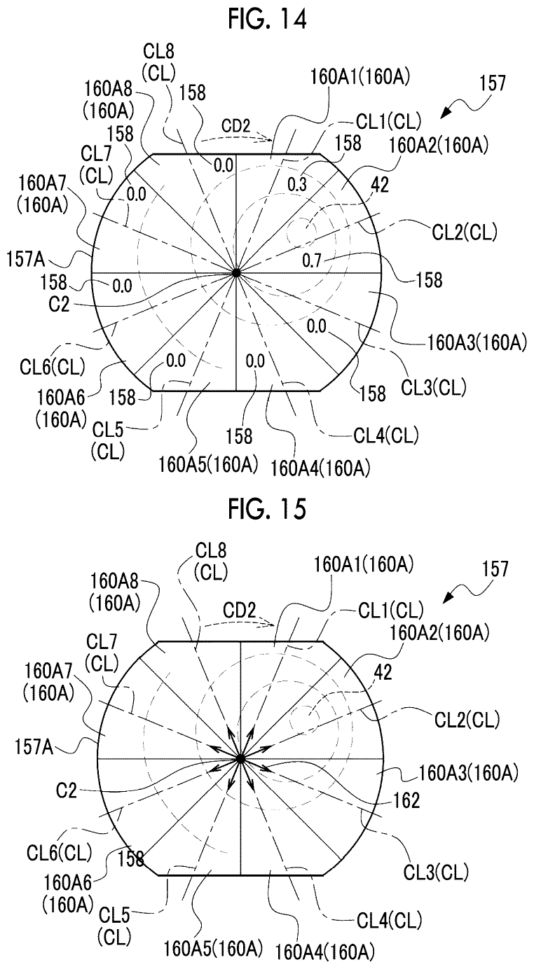

FIG. 14 shows an example of a composition of the confidence level information 157 generated by the lumen recognition model 92 in a case in which the lumen 42 is shown in the frame 40. As shown in FIG. 14, the confidence level information 157 is information including a map 157A corresponding to the frame 40. A size and a shape of the map 157A are the same as a size and a shape of the frame 40. However, this is merely an example, and an outer contour of the map 157A need only be in a similar relationship with an outer contour of the frame 40.

A confidence level 158 (for example, a probability of the existence of the lumen 42) is added to the map 157A. Here, the map 157A is shown as an example, but the frame 40 may be used instead of the map 157A. The map 157A includes a plurality of partition regions 160A corresponding to the plurality of partition regions 130A (see FIGS. 7 to 9). Each of the plurality of partition regions 160A is a region obtained by partitioning the map 157A along a circumferential direction CD2 (in other words, around a center C2 of the map 157A). In the example shown in FIG. 14, partition regions 160A1 to 160A8 are shown as examples of the plurality of partition regions 160A. The partition regions 160A1 to 160A8 are regions obtained by partitioning the map 157A at intervals of a constant angle (for example, at intervals of 45 degrees) along the circumferential direction CD2. In other words, the partition regions 160A1 to 160A8 can be referred to as regions obtained by partitioning the map 157A into eight regions radially from the center C2 of the map 157A toward an outer edge of the map 157A. In other words, the region obtained by the division can also be referred to as a divided region obtained by the division.

The map 157A in the present embodiment is an example of an “image corresponding to the first medical image” and an “image corresponding to the second medical image” according to the present disclosure. In addition, the confidence level 158 in the present embodiment is an example of a “confidence level” and a “weight” according to the present disclosure. In addition, the partition regions 160A1 to 160A8 in the present embodiment are examples of a “plurality of partition regions” and a “plurality of divided regions” according to the present disclosure.

A plurality of center lines CL are provided in the map 157A. The plurality of center lines CL correspond to the plurality of partition regions 160A, and are disposed at equal intervals along the circumferential direction CD2. Each of the plurality of center lines CL is a virtual line along a half angle (for example, 22.5 degrees) of the above-described constant angle from the center C2 in each partition region 160A. In the example shown in FIG. 14, center lines CL1 to CL8 are provided as examples of the plurality of center lines CL for the partition regions 160A1 to 160A8. The center lines CL1 to CL8 are disposed around the center C2 at intervals of 45 degrees.

FIG. 15 shows an aspect example in which a plurality of unit direction vectors 162 are added to the map 157A. As shown in FIG. 15, a direction from the center C2 to the existence position of each of the plurality of partition regions 160A is determined by the unit direction vector 162. The unit direction vector 162 is added to each of the plurality of partition regions 160A. The unit direction vector 162 is a unit vector indicating a direction from the center C2 to the existence position of the partition region 160 (that is, the existence position of each of the partition regions 160A1 to 160A8). In the example shown in FIG. 15, one unit direction vector 162 is added to each of the plurality of partition regions 160A (that is, the partition regions 160A1 to 160A8), along the center line CL for each partition region 160A. The center C2 in the present embodiment is an example of a “reference position” according to the present disclosure. In addition, the plurality of unit direction vectors 162 in the present embodiment are examples of a “plurality of first vectors” according to the present disclosure.

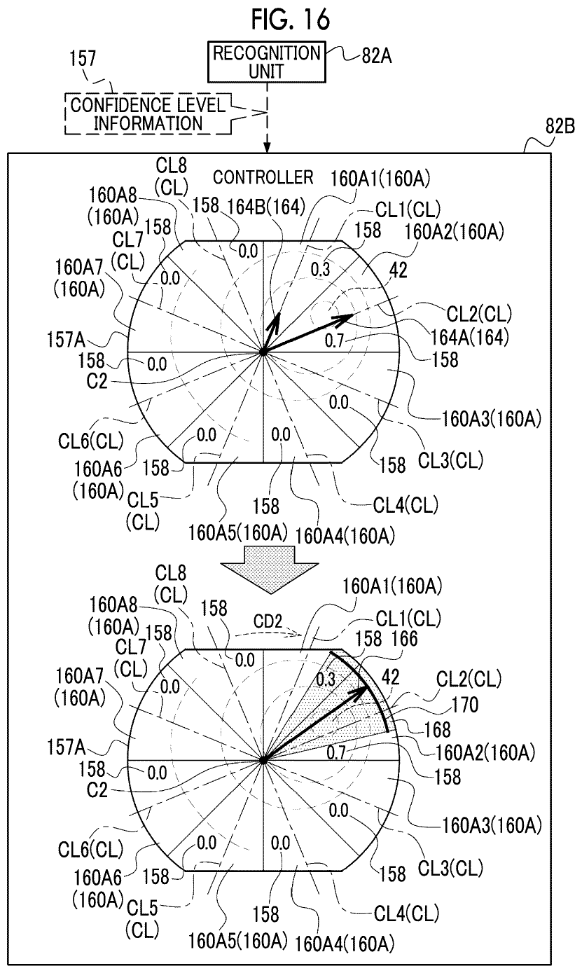

FIG. 16 shows an aspect example in which a plurality of direction vectors 164 are added to the map 157A. As shown in FIG. 16, in a case in which a within-reliability-range condition described later is satisfied, the controller 82B acquires the confidence level information 157 including the map 157A to which the plurality of unit direction vectors 162 (see FIG. 15) are added, from the recognition unit 82A.

The controller 82B generates the plurality of direction vectors 164 based on the plurality of unit direction vectors 162 and the plurality of confidence levels 158 included in the confidence level information 157 acquired from the recognition unit 82A. The direction vector 164 is a vector of which the magnitude is adjusted by adding, as a weight, the confidence level 158 of the partition region 160A, to which the unit direction vector 162 is added, to the unit direction vector 162. The magnitude of the direction vector 164 corresponds to the height of the confidence level 158, and the direction vector 164 becomes larger as the confidence level 158 becomes higher. The plurality of direction vectors 164 in the present embodiment are examples of a “plurality of third vectors” according to the present disclosure.

Here, a specific example of a method for generating the direction vector 164 will be described. For example, in the partition region 160A (in the example shown in FIG. 16, the partition region 160A1) to which “0.3” is added as the confidence level 158, a vector obtained by increasing the magnitude of the unit direction vector 162 by 30% is generated as the direction vector 164 (in the example shown in FIG. 16, the direction vector 164B). In addition, for example, in the partition region 160A (in the example shown in FIG. 16, the partition region 160A2) to which “0.7” is added as the confidence level 158, a vector obtained by increasing the magnitude of the unit direction vector 162 by 70% is generated as the direction vector 164 (in the example shown in FIG. 16, the direction vector 164A). In addition, for example, in the partition region 160A (in the example shown in FIG. 16, the partition regions 160A3 to 160A8) to which “0.0” is added as the confidence level 158, the magnitude of the direction vector 164 may be set to “zero”, or the unit direction vector 162 may be used as the direction vector 164 as it is.

The direction vector 164 shown here is merely an example, and a vector obtained by simply multiplying the unit direction vector 162 by the confidence level 158 may be used as the direction vector 164.

In the example shown in FIG. 16, the direction vectors 164A and 164B are shown. The direction vector 164A is a vector obtained by adjusting the magnitude of the unit direction vector 162 added to the partition region 160A2 by the confidence level 158 (0.7 in the example shown in FIG. 16) of the partition region 160A2. The direction vector 164B is a vector obtained by adjusting the magnitude of the unit direction vector 162 added to the partition region 160A1 by the confidence level 158 (0.3 in the example shown in FIG. 16) of the partition region 160A1. The magnitude of the direction vector 164 represents a degree of the probability of the existence of the lumen 42 in the partition region 160A to which the unit direction vector 162 of the direction vector 164 is added. That is, the larger the direction vector 164 is, the higher the probability of the existence of the lumen 42 in the partition region 160A is to which a base unit direction vector 162 of the direction vector 164 is added.

The controller 82B generates a vector sum 166 based on the plurality of direction vectors 164. The vector sum 166 is a sum of the plurality of direction vectors 164. In the example shown in FIG. 16, as the vector sum 166, a vector sum of the direction vector 164A and the direction vector 164B is shown.

A direction from the center C2 to a lumen existence region 168 in the map 157A is determined by the vector sum 166. The lumen existence region 168 refers to a region in which the lumen 42 is shown in the frame 40. The partition region 160A is a region in which the position in the map 157A is constrained, whereas the lumen existence region 168 is a region in which the position is changed depending on the position at which the vector sum 166 is created without the position being constrained in the map 157A, as in the partition region 160A. Further, in the partition region 160A, even in a case in which the lumen 42 exists in the partition region 160A, it is difficult to estimate the existence position of the lumen 42 in the partition region 160A, but, in the lumen existence region 168, since the lumen 42 exists on the line along the vector sum 166, it is easy to estimate the existence position of the lumen 42. Therefore, in the lumen existence region 168, the existence position of the lumen 42 is specified more precisely than in a case in which the existence position of the lumen 42 is specified in the partition region 160A. In other words, the lumen existence region 168 can be said to be a region in which the position at which the lumen 42 is shown in the frame 40 (that is, the existence position of the lumen 42 in the frame 40) can be specified with a higher resolution than in the plurality of partition regions 160A, along the circumferential direction CD2.

The controller 82B specifies the lumen existence region 168 based on the vector sum 166. For example, the controller 82B specifies, as the lumen existence region 168, a region of ±α degrees along the circumferential direction CD2 with one point (for example, an end point) other than a start point of the vector sum 166 as the center. Examples of the ±α degree include ±22.5 degrees. It should be noted that ±22.5 degrees is merely an example, and the range may be narrower or wider than ±22.5 degrees. Further, the α degree may be a fixed value or a variable value that is changed in accordance with an instruction received by the reception device 64 or various conditions (for example, a type of an operation mode of the endoscope system 10).

The controller 82B generates a mark 170 capable of specifying the existence position of the lumen existence region 168 in the map 157A, based on the plurality of partition regions 160A and the plurality of confidence levels 158.

The “existence position of the lumen existence region 168 in the map 157A” in the present embodiment is an example of a “first existence position” according to the present disclosure. Further, the vector sum 166 in the present embodiment is an example of a “second vector” according to the present disclosure. In addition, the lumen existence region 168 in the present embodiment is an example of a “lumen existence region” according to the present disclosure.

The mark 170 is visible information determined based on the vector sum 166 generated based on the plurality of partition regions 160A and the plurality of confidence levels 158. In the example shown in FIG. 16, the shape of the mark 170 is an arc in which the end point of the vector sum 166 is a midpoint. A center of the arc, which is the shape of the mark 170, is the center C2 of the map 157A. The mark 170 indicates a range from one end to the other end of the lumen existence region 168 in the circumferential direction CD2. The mark 170 in the example shown in FIG. 16 is an example of “lumen specification information” and “first lumen specification information” according to the present disclosure.

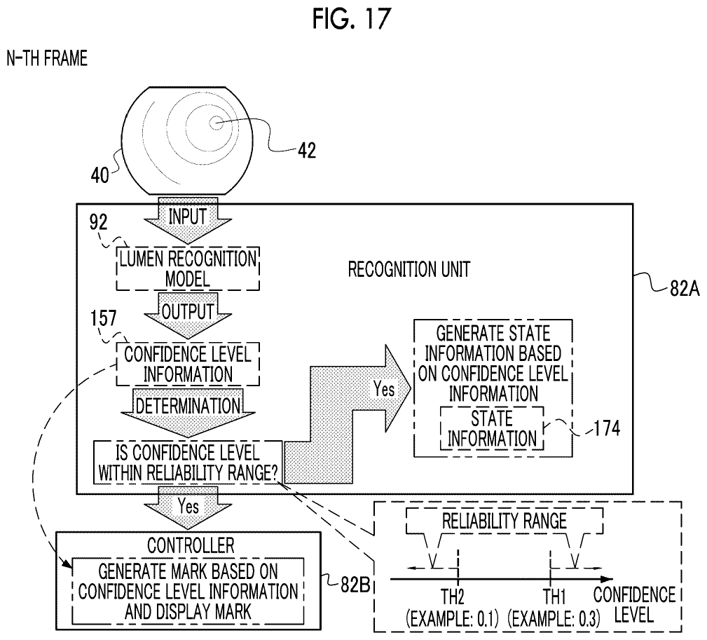

FIG. 17 shows an example of processing contents using an N-th frame 40 in a case in which N is a natural number. In the example shown in FIG. 17, the N-th frame 40 is an example of a “first medical image” according to the present disclosure. As shown in FIG. 17, the recognition unit 82A determines whether or not the within-reliability-range condition is satisfied, which is applied to the confidence level 158 added to each of the plurality of partition regions 160A of the map 157A included in the confidence level information 157 output from the lumen recognition model 92 by inputting the frame 40 (in the example shown in FIG. 17, the N-th frame 40) to the lumen recognition model 92. The within-reliability-range condition refers to a condition in which each of the plurality of confidence levels 158 (here, as an example, all the confidence levels 158) added to the plurality of partition regions 160A of the map 157A included in the confidence level information 157 output from the lumen recognition model 92 by inputting the frame 40 to the lumen recognition model 92 is within a reliability range.

The reliability range refers to a range of the confidence level 158 that is reliable in determining whether or not the lumen 42 exists, the range being derived in advance by a test with an actual machine and/or a computer simulation or the like. The reliability range is defined by a first threshold value TH1 and a second threshold value TH2. The first threshold value TH1 and the second threshold value TH2 are threshold values determined in advance for the confidence level 158. The second threshold value TH2 is a value less than the first threshold value TH1. The reliability range refers to a range equal to or greater than the first threshold value TH1 and a range less than the second threshold value TH2. Here, the first threshold value TH1 is an example of a “first threshold value” and a “third threshold value” according to the present disclosure, and the second threshold value TH2 is an example of a “second threshold value” and a “fourth threshold value” according to the present disclosure.

In the present embodiment, the confidence level 158 in the range equal to or greater than the first threshold value TH1 is a reliable value in a case of determining that the lumen 42 is shown in the partition region 160A, and the confidence level 158 in the range less than the second threshold value TH2 is a reliable value in a case of determining that the lumen 42 is not shown in the partition region 160A. On the other hand, the confidence level 158 in a range that is equal to or greater than the second threshold value TH2 and less than the first threshold value TH1 is set as a value that is not reliable in a case of determining that the lumen 42 is shown in the partition region 160A. Here, the “value that is not reliable in a case of determining that the lumen 42 is shown in the partition region 160A” can also be referred to as a value that triggers the erroneous specification (in other words, erroneous recognition) of the existence position of the lumen 42, which is shown in the frame 40, in the frame 40 via the lumen recognition model 92.

In a case in which the within-reliability-range condition is satisfied, the recognition unit 82A generates state information 174 based on the confidence level information 157. Here, the state information 174 generated based on the confidence level information 157 is an example of “time-series information related to one or more pieces of the first lumen specification information obtained in time series” according to the present disclosure.

The state information 174 means information capable of specifying a state in which the lumen 42 is shown in the partition region 160A (in other words, a state in which the lumen 42 is most likely to be shown in the image region corresponding to the partition region 160A in the entire image region of the frame 40). Examples of the state in which the lumen 42 is shown in the partition region 160A include eight states, that is, ninth to sixteenth states. Therefore, the state information 174 can also be referred to as information capable of specifying which of the ninth to sixteenth states corresponds to the state in which the lumen 42 is shown in the partition region 160A. The ninth state means a state in which the lumen 42 is shown in the partition region 160A1. The tenth state means a state in which the lumen 42 is shown in the partition region 160A2. The eleventh state means a state in which the lumen 42 is shown in the partition region 160A3. The twelfth state means a state in which the lumen 42 is shown in the partition region 160A4. The thirteenth state means a state in which the lumen 42 is shown in the partition region 160A5. The fourteenth state means a state in which the lumen 42 is shown in the partition region 160A6. The fifteenth state means a state in which the lumen 42 is shown in the partition region 160A7. The sixteenth state means a state in which the lumen 42 is shown in the partition region 160A8. It should be noted that, in the examples shown in FIGS. 14 to 16, since the tenth state is shown as a state in which the lumen 42 is shown in the partition region 160A, the recognition unit 82A generates information of capable of specifying the tenth state as the state information 174.

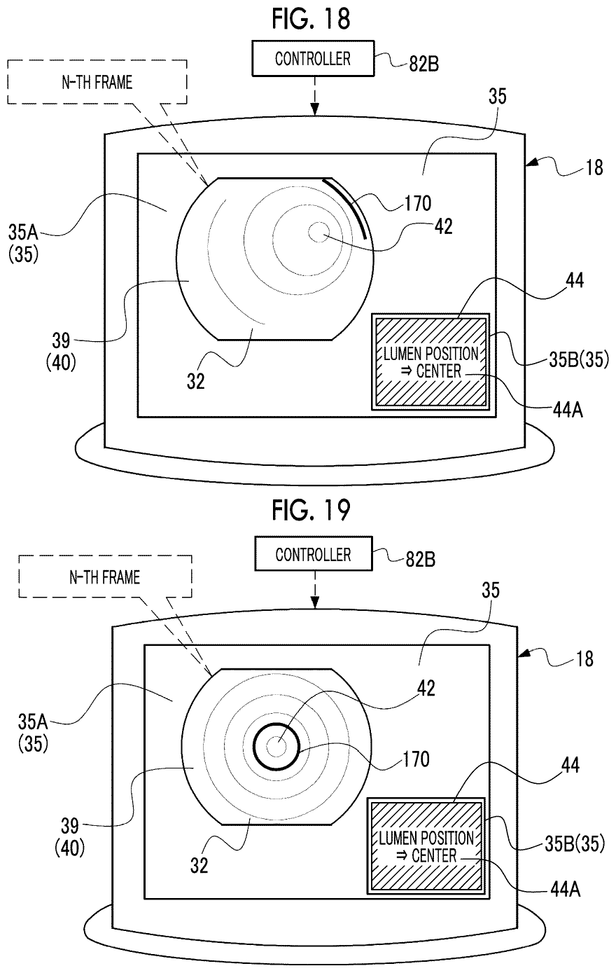

In addition, in a case in which the within-reliability-range condition is satisfied, the controller 82B generates a mark 170 (see FIG. 16) based on the confidence level information 157 and displays the mark 170 in the first display region 35A (see FIGS. 18 and 19). The mark 170 generated based on the confidence level information 157 is information capable of specifying the existence position of the lumen 42, which is shown in the N-th frame 40, in the N-th frame 40. Here, the existence position of the lumen 42, which is shown in the N-th frame 40, in the N-th frame 40 is an example of a “first existence position” according to the present disclosure.

FIG. 18 shows a form example in which various types of information, such as the frame 40 and the mark 170, are displayed on the screen 35 in a case in which the lumen 42 is shown in a region other than a central region of the frame 40. As shown in FIG. 18, the controller 82B displays the frame 40 (in the example shown in FIG. 18, the N-th frame 40) input to the lumen recognition model 92 in order to obtain the confidence level information 157 including the map 157A used to generate the mark 170, in the first display region 35A. Then, the controller 82B displays the mark 170 in the first display region 35A in a state of being comparable with the frame 40. For example, the mark 170 is displayed in a superimposed manner on the frame 40.

Further, the controller 82B updates the mark 170 in accordance with the display of the frame 40. For example, each time the recognition unit 82A determines that the confidence level 158 is within the reliability range (that is, each time the within-reliability-range condition is satisfied), the controller 82B generates the mark 170 based on the confidence level information 157 and displays the mark 170 in a superimposed manner on the frame 40. The mark 170, which is displayed in the first display region 35A, is updated each time the frame 40 is displayed. It should be noted that the mark 170 displayed in the first display region 35A may be updated on the condition that the frame 40 is updated a plurality of times and displayed in the first display region 35A (for example, the frame 40 is displayed in the first display region 35A in a range of a plurality of frames to a plurality of hundreds of frames designated in advance).

Further, the controller 82B displays visible information 44A in the second display region 35B as one piece auxiliary information 44. Examples of the visible information 44A include text capable of specifying a position of a region corresponding to the lumen existence region 168 in the frame 40, that is, text capable of specifying a position of the mark 170 displayed in the first display region 35A (for example, text representing an angle indicating a position of the vector sum 166 in a case in which a boundary line between the partition region 160A1 and the partition region 160A8 is 0 degrees). In addition, in the example shown in FIG. 18, the visible information 44A may include the map 157A or an image based on the map 157A (for example, an image obtained by processing the map 157A) and the mark 170 displayed in a superimposed manner on the map 157A or the image based on the map 157A. The visible information 44A of the example shown in FIG. 18 is an example of “lumen specification information” and “first lumen specification information” according to the present disclosure.

FIG. 19 shows a form example in which the frame 40 and the like are displayed on the screen 35 in a case in which the lumen 42 is shown in the central region of the frame 40 (for example, in a case in which the center of the lumen 42 coincides with the center of the frame 40). As shown in FIG. 19, in a case in which the center of the lumen 42 matches the center of the frame 40, the controller 82B displays the frame 40 (in the example shown in FIG. 19, the N-th frame 40) in the first display region 35A and displays the mark 170 in a superimposed manner on the frame 40. The mark 170 is a mark (for example, an annular mark) surrounding the lumen 42 shown in the frame 40. In a case in which the direction vectors 164 of all the partition regions 160A are equivalent, that is, in a case in which the vector sum 166 is zero, the mark 170 is generated by the controller 82B and is displayed in a superimposed manner on the frame 40. In addition, in this case, the controller 82B displays, as the visible information 44A, information (for example, text) indicating that the lumen 42 is shown at the center of the frame 40 in the second display region 35B. In addition, in the example shown in FIG. 19, the visible information 44A may include the map 157A or the image based on the map 157A (for example, the image obtained by processing the map 157A) and the mark 170 (for example, an annular mark) displayed in a superimposed manner on the central region of the map 157A or the image based on the map 157A. The visible information 44A of the example shown in FIG. 19 is an example of “lumen specification information” and “first lumen specification information” according to the present disclosure.

Here, although the form example has been described in which the mark 170 is generated and is displayed in a superimposed manner on the frame 40 in a case in which the vector sum 166 is zero, this is merely an example. For example, in a case in which the magnitude of the vector sum 166 is less than a threshold value (for example, the magnitude of the unit direction vector 162), the mark 170 may be generated and may be displayed in a superimposed manner on the frame 40. Further, here, as an example of the mark 170, the mark surrounding the lumen 42 shown in the frame 40 has been described, but this is merely an example, and a mark (for example, a dot located at the center of the lumen 42 shown in the frame 40 or an arrow indicating the position of the lumen 42) capable of specifying the position of the lumen 42 shown in the frame 40 may be used.

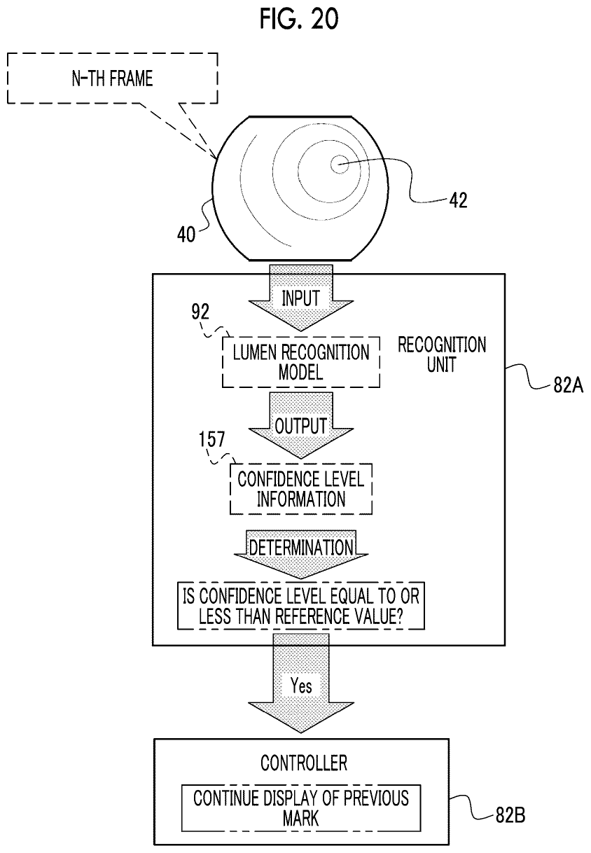

FIG. 20 shows an example of processing contents in a case in which each of the confidence levels 158 added to the plurality of partition regions 160A (here, as an example, all the partition regions 160A) of the map 157A included in the confidence level information 157 output from the lumen recognition model 92 by inputting the N-th frame 40 to the lumen recognition model 92 is equal to or less than a reference value. As shown in FIG. 20, in a case in which each of the confidence levels 158 added to the plurality of partition regions 160A (here, as an example, all the partition regions 160A) of the map 157A included in the confidence level information 157 output from the lumen recognition model 92 by inputting the N-th frame 40 to the lumen recognition model 92 is equal to or less than the reference value, the recognition unit 82A continues the display of the previous mark 170. In the present embodiment, as the reference value, “0.0” is used. Here, “0.0” is an example of a “reference value” according to the present disclosure. It should be noted that “0.0” is merely an example, and a value (for example, 0.05) greater than “0.0” may be used as the reference value instead of “0.0”. It is preferable that the value used as the reference value is a value less than the second threshold value TH2.

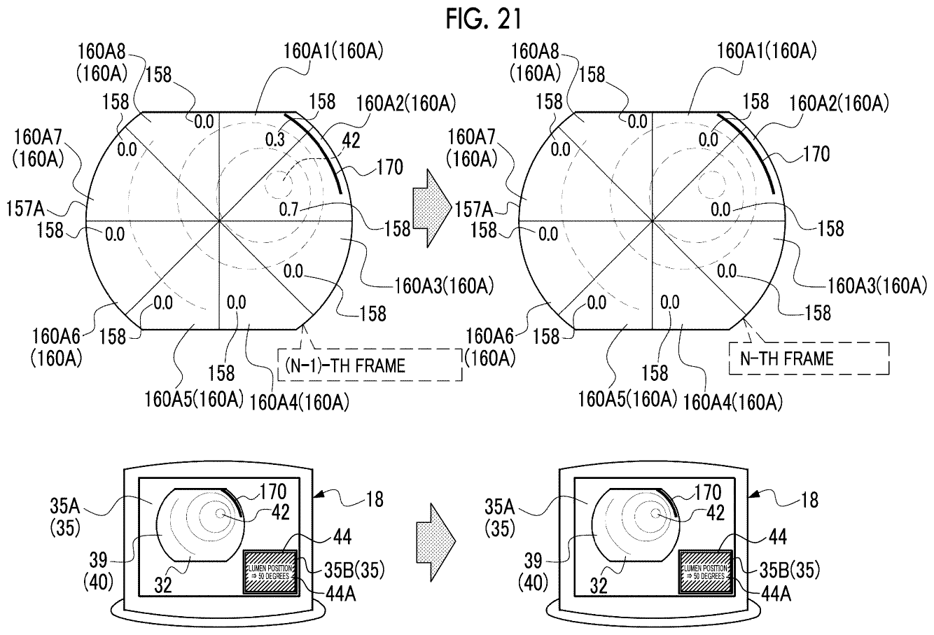

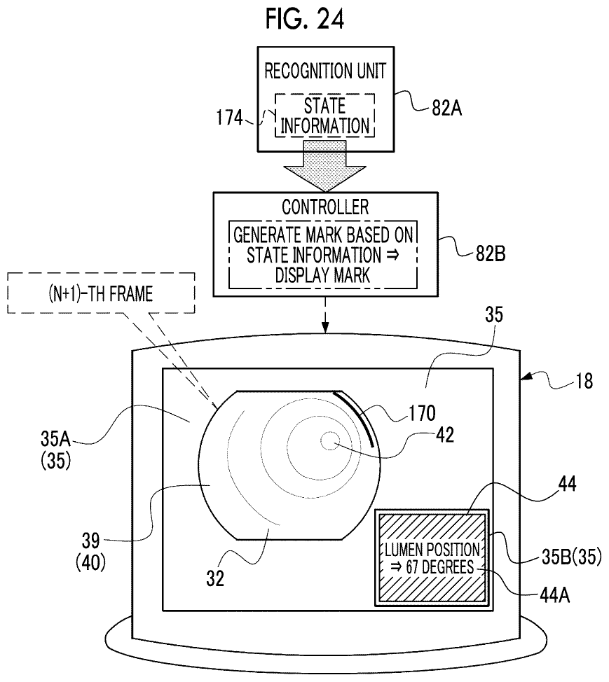

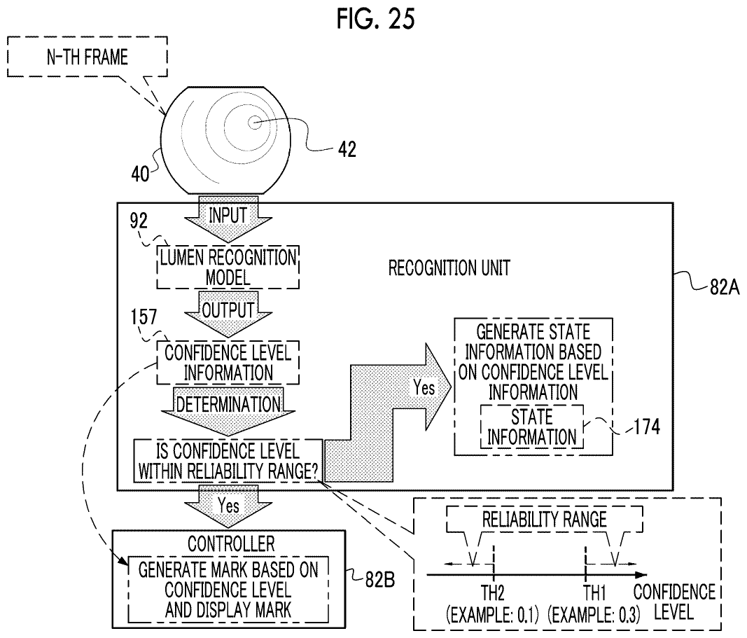

The continuation of the display of the previous mark 170 means the continuation of the display of the mark 170 displayed before the mark 170 shown in FIG. 18 or FIG. 19 is displayed. Examples of the previous mark 170 include the mark 170 generated by the controller 82B in a case in which the same processing as in the example shown in FIG. 17 is performed on an (N−1)-th frame 40 as shown in FIG. 21, or the mark 170 generated by the controller 82B in a case in which the same processing as in the example shown in FIG. 24 or FIG. 25 is performed on the (N−1)-th frame 40. In addition, in a case in which the (N−1)-th frame 40 does not exist, for example, the mark 170 determined by default or the mark 170 generated based on the initial state set in a case of constructing the time-series model 94 is displayed in a superimposed manner on the frame 40.

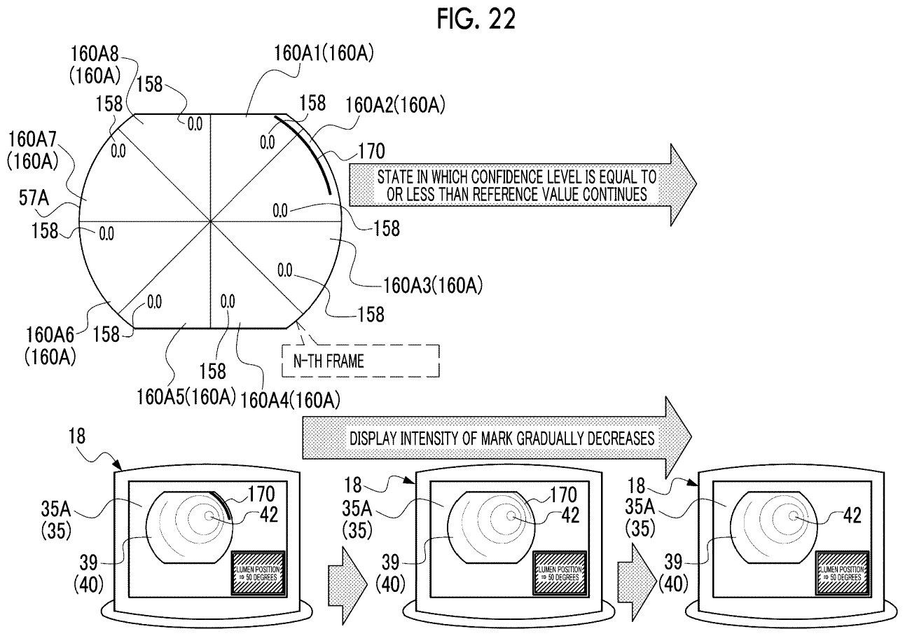

In addition, for example, as shown in FIG. 22, in a case in which a state in which each of the confidence levels 158 added to the plurality of partition regions 160A (here, as an example, all the partition regions 160A) of the map 157A included in the confidence level information 157 output from the lumen recognition model 92 by inputting the N-th frame 40 to the lumen recognition model 92 is equal to or less than the reference value continues for the (N+1)-th and subsequent frames 40, the controller 82B performs control of continuing the display of the mark 170 displayed in a superimposed manner on the N-th frame 40.

In addition, for example, in a case in which a state in which each of the confidence levels 158 added to the plurality of partition regions 160A (here, as an example, all the partition regions 160A) of the map 157A included in the confidence level information 157 output from the lumen recognition model 92 by inputting the N-th frame 40 to the lumen recognition model 92 is equal to or less than the reference value continues for a predetermined period, the controller 82B performs control of gradually decreasing a display intensity (in other words, a display level) of the mark 170, which is displayed before the start of the predetermined period, throughout the predetermined period. Examples of a specific method of gradually decreasing the display intensity include a method of gradually increasing the transparency of the mark 170 and finally hiding the mark 170.

Examples of the predetermined period include a period from the start of the display of the N-th frame 40 to the end of the display of several frames (for example, four frames) 40. Here, the period from the start of the display of the N-th frame 40 to the end of the display of several frames 40 is shown as the predetermined period, but this is merely an example, and the predetermined period may be a period from the start of the display of the N-th frame 40 to the end of the display of several tens of frames 40 or several hundreds of frames 40.

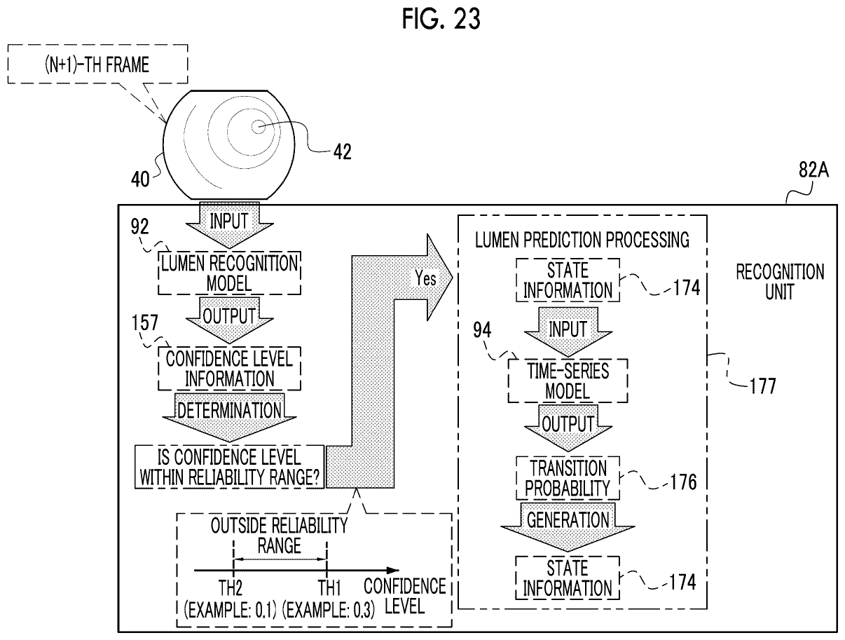

FIG. 23 shows an example of processing contents using an (N+1)-th frame 40 (that is, the frame 40 obtained one frame after the N-th frame 40) in a case in which N is a natural number. In the example shown in FIG. 23, the (N+1)-th frame 40 is an example of a “second medical image” according to the present disclosure. In a case in which the N-th frame 40 of the examples shown in FIGS. 16 to 22 is handled as the (N−1)-th frame 40, the (N+1)-th frame 40 shown in FIG. 23 is handled as the N-th frame 40 of the examples shown in FIGS. 16 to 22.

As shown in FIG. 23, the recognition unit 82A determines whether or not an outside-reliability-range condition is satisfied, the outside-reliability-range condition being applied for each of the confidence levels 158 added to the plurality of partition regions 160A (here, as an example, all the partition regions 160A) of the map 157A included in the confidence level information 157 output from the lumen recognition model 92 by inputting the frame 40 (in the example shown in FIG. 23, the (N+1)-th frame 40) to the lumen recognition model 92. The outside-reliability-range condition means a condition in which each of the confidence levels 158 added to the plurality of partition regions 160A (here, as an example, all the partition regions 160A) of the map 157A included in the confidence level information 157 output from the lumen recognition model 92 by inputting the frame 40 to the lumen recognition model 92 is not within the reliability range. In other words, the outside-reliability-range condition can also be referred to as a condition in which each of the confidence levels 158 added to the plurality of partition regions 160A (here, as an example, all the partition regions 160A) of the map 157A included in the confidence level information 157 output from the lumen recognition model 92 by inputting the frame 40 to the lumen recognition model 92 is outside the reliability range. In other words, the outside-reliability-range condition can also be referred to as a condition in which the within-reliability-range condition is not satisfied.

The outside-reliability-range condition is a condition related to the reliability of each of the confidence levels 158 for the plurality of partition regions 160A (here, as an example, all the partition regions 160A). In the present embodiment, examples of the outside-reliability-range condition include a condition in which each of the confidence levels 158 for the plurality of partition regions 160A (here, as an example, all the partition regions 160A) is equal to or greater than the second threshold value TH2 and less than the first threshold value TH1. The outside-reliability-range condition in the present embodiment is an example of an “erroneous specification triggering condition” and a “confidence level condition” according to the present disclosure.

In a case in which the recognition unit 82A determines that the outside-reliability-range condition is satisfied, the recognition unit 82A executes lumen prediction processing 177. The lumen prediction processing 177 is processing using the time-series model 94, in which the existence position of the lumen 42, which is shown in the (N+1)-th frame 40, in the (N+1)-th frame 40 is predicted.

In the lumen prediction processing 177, latest state information 174 (that is, information capable of specifying a current state among the ninth to sixteenth states) obtained in a case in which the frame 40 obtained before the (N+1)-th frame is set as a processing target is input to the time-series model 94. A first example of the latest state information 174 is the state information 174 generated by executing the processing shown in FIG. 17. A second example of the latest state information 174 is the state information 174 generated by executing the processing shown in FIG. 23 on the N-th frame 40. The time-series model 94 outputs a transition probability 176 in response to the input of the latest state information 174. Here, although the form example has been described in which the time-series model 94 outputs the transition probability 176 in response to the input of one piece of state information 174, this is merely an example. For example, in a case in which the Markov model based on the N-th order Markov chain is used instead of the time-series model 94, the Markov model may output the transition probability 176 in response to the input of a plurality of pieces of state information 174 that were previously obtained. The transition probability 176 output from the time-series model 94 in the present embodiment is an example of “information obtained from the time-series model” according to the present disclosure.

The transition probability 176 output from the time-series model 94 is a probability of transition from a state (for example, the tenth state in the examples shown in FIGS. 14 to 16) specified from the state information 174 input to the time-series model 94 to a next state. In the lumen prediction processing 177, the state information 174 is generated based on the transition probability 176 output from the time-series model 94. The time-series model 94 outputs a plurality of transition probabilities 176. In the lumen prediction processing 177, information capable of specifying a state corresponding to a maximum transition probability 176 among the plurality of transition probabilities 176 output from the time-series model 94 is generated as the state information 174. Here, the state specified from the generated state information 174 is a next state (that is, a state in which the lumen 42 is shown in the partition region 160A used for the (N+1)-th frame 40) that transitions from the state specified from the state information 174 input to the time-series model 94. The next state that transitions from the state specified from the state information 174 input to the time-series model 94 is any one of the ninth to sixteenth states.

FIG. 24 shows an example of an aspect in which the state specified from the state information 174 generated by executing the lumen prediction processing 177 by the recognition unit 82A is visualized on the screen 35. As shown in FIG. 24, the controller 82B displays the frame 40 (in the example shown in FIG. 24, the (N+1)-th frame 40) in the first display region 35A by the same method as in the example shown in FIG. 18. In addition, in a case in which the outside-reliability-range condition is satisfied, the controller 82B acquires the state information 174 generated by executing the lumen prediction processing 177 via the recognition unit 82A from the recognition unit 82A. The controller 82B generates the mark 170 based on the state information 174 acquired from the recognition unit 82A. The mark 170 generated based on the state information 174 is a mark capable of specifying the state specified from the state information 174 (that is, any state of the ninth to sixteenth states). In other words, the mark 170 generated based on the state information 174 can also be referred to as information capable of specifying the existence position of the lumen 42, which is shown in the (N+1)-th frame 40, in the (N+1)-th frame 40. Here, the existence position of the lumen 42, which is shown in the (N+1)-th frame 40, in the (N+1)-th frame 40 is an example of a “second existence position” according to the present disclosure.

The controller 82B displays the mark 170 generated based on the state information 174 obtained from the time-series model 94 in a case in which the outside-reliability-range condition is satisfied, in the first display region 35A in a state of being comparable with the frame 40. For example, the mark 170 is displayed in a superimposed manner on the frame 40. Further, the mark 170 is displayed at the position of the partition region 160A corresponding to the state specified from the state information 174. For example, in a case in which the state specified from the state information 174 is the tenth state, the mark 170 is displayed at a position corresponding to the partition region 160A2. As a result, the existence position of the lumen 42, which is shown in the (N+1)-th frame 40, in the (N+1)-th frame 40 is visually specified from the display position of the mark 170.

Further, the controller 82B updates the mark 170 in accordance with the display of the frame 40. For example, the controller 82B generates the mark 170 based on the state information 174 and displays the mark 170 in a superimposed manner on the frame 40 each time the state information 174 is obtained by executing the lumen prediction processing 177. In a case in which the mark 170 is generated based on the state information 174, the visible information 44A is displayed in the second display region 35B by the same method as in the example shown in FIG. 18. In the example shown in FIG. 24, the mark 170 generated based on the state information 174 and displayed in the first display region 35A is an example of “lumen specification information” and “second lumen specification information” according to the present disclosure.

FIG. 25 shows an example of processing contents in a case in which the within-reliability-range condition is satisfied (that is, in a case in which each of the confidence levels 158 added to the partition regions 160A of the map 157A included in the confidence level information 157 output from the lumen recognition model 92 is within the reliability range). As shown in FIG. 25, in a case in which the within-reliability-range condition is satisfied, the recognition unit 82A and the controller 82B perform the same processing as in the example shown in FIG. 17. In this case, the controller 82B generates the mark 170 (see FIG. 16) based on the confidence level information 157 output from the lumen recognition model 92 by inputting the (N+1)-th frame 40 to the lumen recognition model 92. Then, the controller 82B displays the mark 170 in the first display region 35A by the same method as in the example shown in FIG. 18. The mark 170 generated based on the confidence level information 157 output from the lumen recognition model 92 by inputting the (N+1)-th frame 40 to the lumen recognition model 92 is information capable of specifying the existence position of the lumen 42, which is shown in the (N+1)-th frame 40, in the (N+1)-th frame 40.

The mark 170 generated based on the confidence level information 157 output from the lumen recognition model 92 by inputting the (N+1)-th frame 40 to the lumen recognition model 92 in the present embodiment is an example of “lumen specification information” and “third lumen specification information” according to the present disclosure. In addition, the existence position of the lumen 42, which is shown in the (N+1)-th frame 40, in the (N+1)-th frame 40 in the present embodiment is an example of a “third existence position” according to the present disclosure.

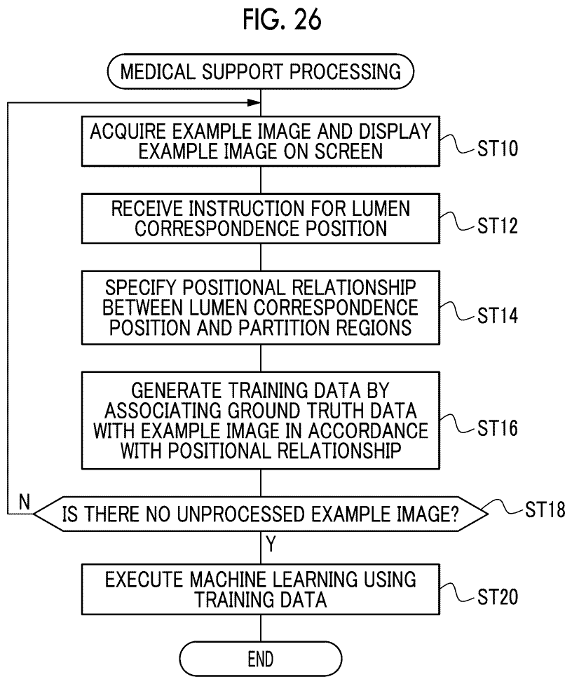

Hereinafter, an example of a flow of the machine learning processing performed by the information processing device 100 will be described with reference to FIG. 26.

In the machine learning processing shown in FIG. 26, first, in step ST10, the training data generation unit 106A acquires an unprocessed example image 122A from the example image set 122 (see FIGS. 5 and 6) stored in the storage 110. Here, the unprocessed example image 122A means the example image 122A that has not yet been used for the machine learning processing. The training data generation unit 106A displays the example image 122A, which is acquired from the example image set 122, on the screen 118A (see FIGS. 6, 8, and 9). After the processing of step ST10 is executed, the machine learning processing proceeds to step ST12.

In step ST12, the training data generation unit 106A receives an instruction for the lumen correspondence position 139 (see FIGS. 8 and 9). After the processing of step ST12 is executed, the machine learning processing proceeds to step ST14.

In step ST14, the training data generation unit 106A specifies a positional relationship between the lumen correspondence position 139 received in step ST12 and the plurality of partition regions 130A (see FIGS. 8 and 9). After the processing of step ST14 is executed, the machine learning processing proceeds to step ST16.

In step ST16, the training data generation unit 106A associates the ground truth data 126 with the example image 122A acquired in step ST10 in accordance with the positional relationship specified in step ST14 (see FIGS. 8 and 9). For example, in a case in which the lumen correspondence position 139 exists in a region other than the central region of the example image 122A, the ground truth data 126 is associated with the partition region 130A having the largest overlap area with the lumen correspondence position 139 in response to the instruction issued from the annotator 124. Further, for example, in a case in which the lumen correspondence position 139 exists in the central region of the example image 122A, the ground truth data 126 is associated with each of the partition regions 130A in response to the instruction issued from the annotator 124. In this way, the training data generation unit 106A associates the ground truth data 126 with the example image 122A to generate the training data 128. The training data 128 generated in this manner is stored in a predetermined storage medium (for example, the storage 110). After the processing of step ST16 is executed, the machine learning processing proceeds to step ST18.

In step ST18, the training data generation unit 106A determines whether or not there is no unprocessed example image 122A. In step ST18, in a case in which there is an unprocessed example image 122A, a negative determination is made, and the machine learning processing proceeds to step ST10. In step ST18, in a case in which there is no unprocessed example image 122A, an affirmative determination is made, and the machine learning processing proceeds to step ST20.

In step ST20, the learning execution unit 106B executes machine learning using the plurality of pieces of training data 128 obtained by repeatedly executing the processing in steps ST10 to ST18 to generate the lumen recognition model 92 (see FIG. 10). The lumen recognition model 92 is stored in the storage 86 of the medical support device 24 (see FIG. 4). After the processing of step ST20 is executed, the machine learning processing ends.

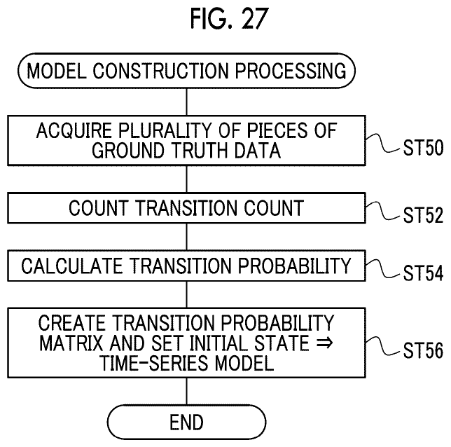

Hereinafter, an example of a flow of the model construction processing performed by the information processing device 100 will be described with reference to FIG. 27.

In the model construction processing shown in FIG. 27, first, in step ST50, the model construction unit 106C acquires, in time series, the plurality of pieces of ground truth data 152A2 associated with a plurality of time-series images 152A1 arranged in time series from the time-series dataset 152A stored in the storage 110 (see FIG. 12). After the processing of step ST50 is executed, the model construction processing proceeds to step ST52.

In step ST52, the model construction unit 106C counts the transition count 154 between the respective states in which the lumen 138 is shown in the partition region 130A in the plurality of time-series images 152A1 arranged in time series from the plurality of pieces of ground truth data 152A2 arranged in time series (see FIG. 12). After the processing of step ST52 is executed, the model construction processing proceeds to step ST54.

In step ST54, the model construction unit 10C calculates the transition probability 156 based on the transition count 154 (see FIG. 12). After the processing of step ST54 is executed, the model construction processing proceeds to step ST56.

In step ST56, the model construction unit 106C constructs the time-series model 94 by creating the transition probability matrix based on the transition probability 156 and setting the initial state (see FIG. 12). After the processing of step ST56 is executed, the model construction processing ends.

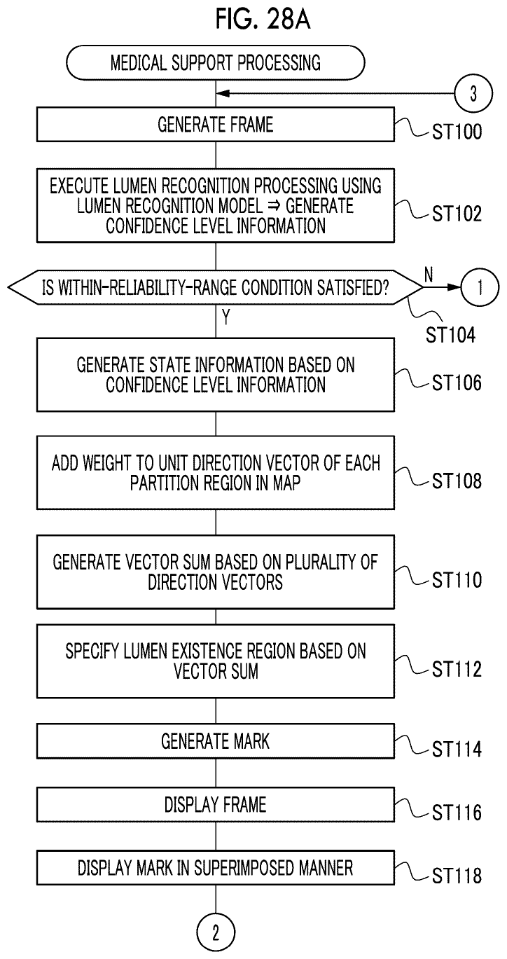

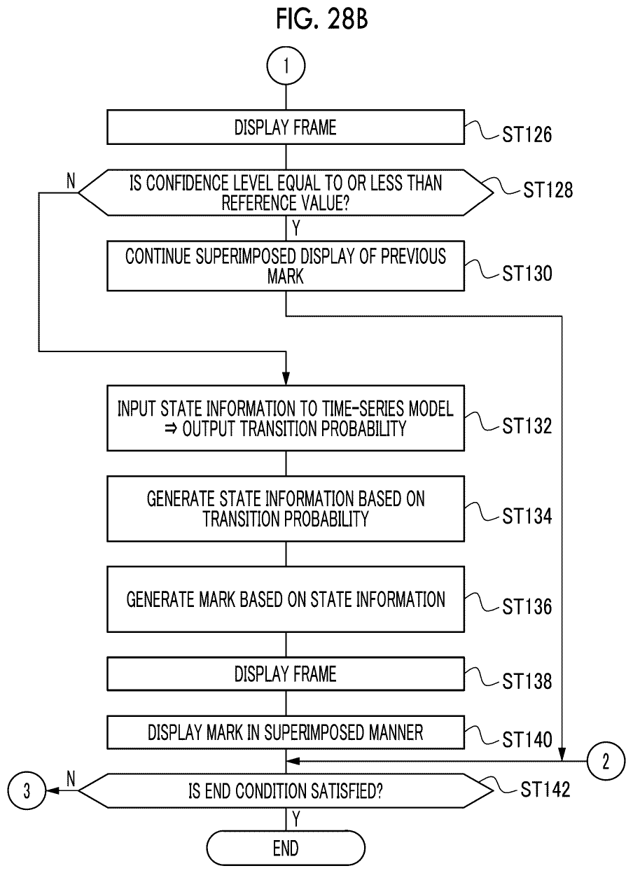

Hereinafter, an example of a flow of the medical support processing performed by the endoscope system 10 will be described with reference to FIGS. 28A and 28B. The flow of the medical support processing shown in FIGS. 28A and 28B is an example of a “medical support method” according to the present disclosure. Hereinafter, for convenience, the description will be made on the assumption that the lumen recognition model 92 generated by executing the machine learning processing and the time-series model 94 constructed by executing the model construction processing are stored in the storage 86, and the lumen recognition model 92 and the time-series model 94 are acquired from the storage 86 by the recognition unit 82A and used.

In the medical support processing shown in FIG. 28A, first, in step ST100, the recognition unit 82A acquires the image 151 from the camera 52, and performs various types of processing on the acquired image 151 to generate the frame 40 (see FIG. 13). After the processing in step ST100 is executed, the medical support processing proceeds to step ST102.

In step ST102, the recognition unit 82A executes the lumen recognition processing 153 via the lumen recognition model 92 to cause the lumen recognition model 92 to generate the confidence level information 157 (see FIGS. 13, 17, 23, and 25). After the processing in step ST102 is executed, the medical support processing proceeds to step ST104.

In step ST104, the recognition unit 82A determines whether or not the within-reliability-range condition is satisfied. That is, the recognition unit 82A determines whether or not each of the confidence levels 158 included in the confidence level information 157 generated in step ST102 is within the reliability range (see FIGS. 17, 23, and 25). In step ST104, in a case in which the within-reliability-range condition is not satisfied (that is, in a case in which the outside-reliability-range condition is satisfied), a negative determination is made, and the medical support processing proceeds to step ST126 shown in FIG. 28B. In step ST104, in a case in which the within-reliability-range condition is satisfied, an affirmative determination is made, and the medical support processing proceeds to step ST106.

In step ST106, the recognition unit 82A generates the state information 174 based on the confidence level information 157 generated by the lumen recognition model 92 (see FIGS. 17 and 25). Here, examples of the generated state information 174 include information capable of specifying a state in which the lumen 42 is shown in the partition region 160A to which the maximum confidence level 158 is added among all the confidence levels 158 added to all the partition regions 160A of the map 157A included in the confidence level information 157. After the processing in step ST106 is executed, the medical support processing proceeds to step ST108.

In step ST108, the controller 82B generates the plurality of direction vectors 164 by adding, as a weight, the confidence level 158 of the partition region 160A, to which each unit direction vector 162 is added, to the unit direction vector 162 of each partition region 160A of the map 157A included in the confidence level information 157 generated in step ST102 (see FIG. 16). After the processing in step ST108 is executed, the medical support processing proceeds to step ST110.

In step ST110, the controller 82B generates the vector sum 166 which is a sum of the plurality of direction vectors 164 generated in step ST108 (see FIG. 16). After the processing in step ST110 is executed, the medical support processing proceeds to step ST112.

In step ST112, the controller 82B specifies the lumen existence region 168 based on the vector sum 166 generated in step ST110. After the processing in step ST112 is executed, the medical support processing proceeds to step ST114.

In step ST114, the controller 82B generates the mark 170 capable of specifying the position of the lumen existence region 168 specified in step ST112 (see FIG. 16). After the processing in step ST114 is executed, the medical support processing proceeds to step ST116.

In step ST116, the controller 82B displays the frame 40, which is generated in step ST100, in the first display region 35A (see FIGS. 18 and 19). After the processing in step ST116 is executed, the medical support processing proceeds to step ST118.

In step ST118, the controller 82B displays the mark 170, which is generated in step ST114, in a superimposed manner on the frame 40 displayed in the first display region 35A (see FIGS. 18 and 19). After the processing in step ST118 is executed, the medical support processing proceeds to step ST142 shown in FIG. 28B.

In step ST126 shown in FIG. 28B, the controller 82B displays the frame 40, which is generated in step ST100, in the first display region 35A. After the processing in step ST126 is executed, the medical support processing proceeds to step ST128.

In step ST128, the controller 82B determines whether or not each of the confidence levels 158 added to the partition regions 160A of the map 157A included in the confidence level information 157 generated in step ST102 is equal to or less than the reference value (see FIG. 20). In step ST128, in a case in which each of the confidence levels 158 added to the partition regions 160A of the map 157A included in the confidence level information 157 generated in step ST102 is equal to or less than the reference value, an affirmative determination is made, and the medical support processing proceeds to step ST130. In step ST128, in a case in which each of the confidence levels 158 added to the partition regions of the map 157A included in the confidence level information 157 generated in step ST102 is not equal to or less than the reference value, a negative determination is made, and the medical support processing proceeds to step ST132.

In step ST130, the controller 82B continues the superimposed display of the previous mark 170 (see FIGS. 21 and 22). For example, the controller 82B continues the display of the mark 170 displayed in a superimposed manner on the preceding frame 40. After the processing in step ST130 is executed, the medical support processing proceeds to step ST142.

In step ST132, the recognition unit 82A executes the lumen prediction processing 177 using the latest state information 174. That is, the recognition unit 82A inputs the latest state information 174 to the time-series model 94 to cause the time-series model 94 to output the transition probability 176 (see FIG. 23). Here, examples of the latest state information 174 include the state information 174 (see FIG. 17) generated in a case in which the processing shown in FIG. 17 is performed on the preceding frame 40, or the state information 174 (see FIG. 23) generated in a case in which the lumen prediction processing 177 shown in FIG. 23 is performed on the preceding frame 40. The state information 174 generated in a case in which the processing shown in FIG. 17 is performed on the preceding frame 40 refers to the state information 174 generated by executing the processing of step ST106 in a case in which the preceding frame 40 is set as the processing target. In addition, the state information 174 generated in a case in which the lumen prediction processing 177 shown in FIG. 23 is performed on the preceding frame 40 refers to the state information 174 generated by executing the processing of step ST134 in a case in which the preceding frame 40 is set as the processing target. After the processing in step ST132 is executed, the medical support processing proceeds to step ST134.

In step ST134, the recognition unit 82A generates the state information 174 based on the transition probability 176 output from the time-series model 94 in step ST132 (see FIG. 23). After the processing in step ST134 is executed, the medical support processing proceeds to step ST136.

In step ST136, the controller 82B generates the mark 170 based on the state information 174 generated in step ST134 (see FIG. 24). After the processing in step ST136 is executed, the medical support processing proceeds to step ST138.

In step ST138, the controller 82B displays the frame 40, which is generated in step ST100, in the first display region 35A (see FIG. 24). After the processing in step ST138 is executed, the medical support processing proceeds to step ST140.

In step ST140, the controller 82B displays the mark 170, which is generated in step ST136, in a superimposed manner on the frame 40 displayed in the first display region 35A (see FIG. 24). After the processing in step ST140 is executed, the medical support processing proceeds to step ST142.

In step ST142, the controller 82B determines whether or not a medical support processing end condition is satisfied. Examples of the medical support processing end condition include a condition in which an instruction to end the medical support processing is issued to the endoscope system 10 (for example, a condition in which the reception device 64 receives the instruction to end the medical support processing).

In step ST142, in a case in which the medical support processing end condition is not satisfied, a negative determination is made, and the medical support processing proceeds to step ST100 shown in FIG. 28A. In a case in which the medical support processing end condition is satisfied in step ST100, an affirmative determination is made, and the medical support processing ends.

As described above, in the endoscope system 10, as the information capable of specifying the existence position of the lumen 42, which is shown in the frame 40, in the frame 40, the mark 170 (hereinafter, also referred to as a “first mark” without a reference numeral) generated based on the confidence level information 157 obtained by the lumen recognition processing 153 using the lumen recognition model 92, and the mark 170 (hereinafter, also referred to as a “second mark” without a reference numeral) generated based on the state information 174 obtained by the lumen prediction processing 177 using the time-series model 94 are selectively displayed in the first display region 35A. Here, the first mark is generated based on the confidence level information 157 obtained from the lumen recognition model 92 by inputting the N-th frame 40 to the lumen recognition model 92, and is a mark capable of specifying the existence position of the lumen 42, which is shown in the N-th frame 40, in the N-th frame 40. In addition, the second mark is generated based on the transition probability 176 obtained from the time-series model 94 by inputting information (for example, the state information 174 generated based on the confidence level information 157 used to generate the first mark) related to the first mark to the time-series model 94, and is a mark capable of specifying the existence position of the lumen 42, which is shown in the (N+1)-th frame 40, in the (N+1)-th frame 40. Therefore, with the endoscope system 10, the doctor 12 or the like can ascertain the existence position of the lumen 42, which is shown in each of the plurality of frames 40 arranged in time series (for example, the N-th frame 40 and the (N+1)-th frame 40), in each frame 40 without omission.

In addition, in the endoscope system 10, the mark 170 is displayed in the first display region 35A in a state of being comparable with the frame 40 displayed in the first display region 35A. For example, the mark 170 is displayed in a superimposed manner on the frame 40 displayed in the first display region 35A. As a result, the doctor 12 or the like can visually ascertain the positional relationship between a portion in the large intestine 28 observed through the frame 40 and the lumen 42.

Further, in the endoscope system 10, the mark 170 displayed in the first display region 35A is updated in accordance with the display of the frame 40. As a result, it is possible to ensure the consistency between the content of the frame 40 displayed in the first display region 35A and the mark 170, and it is possible for the doctor 12 or the like to visually recognize the mark 170 in accordance with the content shown in the frame 40 displayed in the first display region 35A.

In addition, in the endoscope system 10, in a case in which the frame 40 displayed in the first display region 35A is an image that satisfies the outside-reliability-range condition, the second mark is displayed in the first display region 35A. Therefore, even in a case in which the frame 40 displayed in the first display region 35A is an image that satisfies the outside-reliability-range condition, the doctor 12 or the like can visually ascertain the existence position

of the lumen 42 shown in the frame 40.

In addition, in the endoscope system 10, in a case in which the frame 40 displayed in the first display region 35A is an image that does not satisfy the outside-reliability-range condition (that is, an image that satisfies the within-reliability-range condition), a third mark is generated, and the third mark is displayed in a superimposed manner on the frame 40 displayed in the first display region 35A. The third mark is generated based on the confidence level information 157 obtained from the lumen recognition model 92 by inputting the (N+1)-th frame 40 to the lumen recognition model 92. The confidence level information 157 includes the map 157A partitioned into the plurality of partition regions 160A. The confidence level 158 indicating that the lumen 42 exists is added to each of the plurality of partition regions 160A. In addition, the third mark is a mark 170 capable of specifying the existence position of the lumen 42, which is in the (N+1)-th frame 40, in the (N+1)-th frame 40. Therefore, even in a case in which the frame 40 displayed in the first display region 35A is an image that does not satisfy the outside-reliability-range condition (that is, an image that satisfies the within-reliability-range condition), the doctor 12 or the like can visually ascertain the existence position of the lumen 42 shown in the frame 40.

In addition, in the endoscope system 10, in a case in which all the confidence levels 158 included in the confidence level information 157 output from the lumen recognition model 92 by inputting the N-th frame 40 to the lumen recognition model 92 are equal to or less than the reference value, the display of the mark 170 displayed in the first display region 35A before the confidence level 158 equal to or less than the reference value is generated is continued. In addition, in the endoscope system 10, in a case in which a state in which all the confidence levels 158 included in the confidence level information 157 output from the lumen recognition model 92 by inputting the N-th frame 40 to the lumen recognition model 92 are equal to or less than the reference value continues for a predetermined period (for example, several frames or a time required for displaying several frames), the display level of the mark 170 displayed in the first display region 35A before the start of the predetermined period gradually decreases throughout the predetermined period. In this way, it is possible to reduce the visual discomfort given to the doctor 12 or the like due to the switching between the display and the hiding of the mark 170 (for example, flickering due to the switching between the display and the hiding of the mark 170), as compared to a case in which the display and the hiding of the mark 170 are frequently switched.

Further, in the endoscope system 10, the frame 40 in which the intestinal wall 32 and the lumen 42 are shown is input to the lumen recognition model 92, so that the confidence level information 157 is generated by the lumen recognition model 92. The confidence level information 157 includes the map 157A partitioned into the plurality of partition regions 160A. The confidence level 158 indicating that the lumen 42 exists is added to each of the plurality of partition regions 160A.

In the endoscope system 10, the lumen existence region 168 is generated based on the plurality of partition regions 160A and the plurality of confidence levels 158. The lumen existence region 168 is a region in which the existence position of the lumen 42 (that is, the position at which the lumen 42 is shown) is specified more precisely than in the partition region 160A in the frame 40. Since the lumen existence region 168 is generated based on the plurality of partition regions 160A and the plurality of confidence levels 158, the position of the lumen existence region 168 is not fixed in the map 157A as in the partition region 160A. In addition, the position of the lumen existence region 168 in the map 157A is finely changed along the circumferential direction CD2 depending on the existence position of the lumen 42. This means that the lumen existence region 168 is a region in which the existence position of the lumen 42 is defined with a higher resolution than in the plurality of partition regions 160A, along the circumferential direction CD2.

In the endoscope system 10, the mark 170 is generated as information capable of specifying the lumen existence region 168. Then, the frame 40 input to the lumen recognition model 92 for generating the confidence level information 157 is displayed in the first display region 35A of the screen 35. Further, the mark 170 is displayed in a superimposed manner on the frame 40. This means that the mark 170 is represented on the frame 40 with a higher resolution than in a case in which the confidence level 158 of each of the plurality of partition regions 160A is simply displayed in a superimposed manner on the frame 40 or the visible information (for example, the mark) indicating the height of the confidence level 158 is simply displayed in the first display region 35A.

Therefore, the doctor 12 can more accurately ascertain the position of the lumen 42, which is shown in the frame 40, in the frame 40 by simply visually recognizing the mark 170 displayed in a superimposed manner on the frame 40, as compared with a case in which the confidence level 158 of each of the plurality of partition regions 160A is simply displayed in a superimposed manner on the frame 40 or the visible information indicating the height of the confidence level 158 is simply displayed in the first display region 35A.

In addition, in the endoscope system 10, the direction from the center C2 of the map 157A to the existence position of each of the plurality of partition regions 160A is determined by the plurality of unit direction vectors 162. In addition, the direction from the center C2 of the map 157A to the lumen existence region 168 is determined by the vector sum 166. The vector sum 166 is a sum of the plurality of direction vectors 164 obtained by adding, as the weight, the confidence level 158 to the plurality of unit direction vectors 162. The mark 170, which is displayed in a superimposed manner on the frame 40, is generated based on the vector sum 166.

Here, the plurality of direction vectors 164 are changed in accordance with the confidence level 158 added to each of the plurality of partition regions 160A. The vector sum 166 is changed in accordance with the plurality of direction vectors 164. It can be said that the vector sum 166 is a vector indicating the direction from the center C2 of the map 157A to the existence position of the lumen 42. It can be said that the mark 170 generated based on the vector sum 166 is visible information that represents the existence position of the lumen 42, which is shown in the frame 40, in the frame 40 more precisely as compared with a case in which the confidence level 158 of each of the plurality of partition regions 160A is simply displayed in a superimposed manner on the frame 40 or the visible information indicating the height of the confidence level 158 is simply displayed in the first display region 35A.

Therefore, the doctor 12 can more accurately ascertain the position of the lumen 42, which is shown in the frame 40, in the frame 40 by simply visually recognizing the mark 170 displayed in a superimposed manner on the frame 40, as compared with a case in which the confidence level 158 of each of the plurality of partition regions 160A is simply displayed in a superimposed manner on the frame 40 or the visible information indicating the height of the confidence level 158 is simply displayed in the first display region 35A.

In the above-described embodiment, the form example has been described in which the lumen existence region 168 is generated based on the plurality of partition regions 160A and the plurality of confidence levels 158 (see FIG. 16), but the present disclosure is not limited to this. For example, by the same method as in a case in which the lumen existence region 168 is generated based on the plurality of partition regions 160A and the plurality of confidence levels 158, the lumen existence region 168 may be generated based on the plurality of partition regions 160A applied to the (N+1)-th frame 40 (see FIG. 23) and the plurality of transition probabilities 176 (see FIG. 23) output from the time-series model 94. In this case as well, the mark 170 may be generated by the same method as in the example shown in FIG. 16, and the generated mark 170 may be displayed in the first display region 35A by the same method as in the example shown in FIG. 18.

In the above-described embodiment, although the form example has been described in which it is determined whether or not the within-reliability-range condition is satisfied in step ST104 shown in FIG. 28A, this is merely an example, and it is sufficient that it is determined whether or not the erroneous specification triggering condition, which is a condition that triggers the erroneous specification (in other words, erroneous recognition) via the lumen recognition model 92, is satisfied. Here, the erroneous specification refers to the erroneous specification of the existence position of the lumen 42, which is shown in the frame 40 (for example, the (N+1)-th frame 40) input to the lumen recognition model 92, in the frame 40.

The erroneous specification triggering condition is a condition including at least one of the first to fifth conditions. The first condition is a condition in which at least a part of the lumen 42 is not shown in the frame 40 (for example, the (N+1)-th frame 40). The second condition is a condition in which at least one dark portion different from the lumen 42 is shown in the frame 40 (for example, the (N+1)-th frame 40). The “dark portion” here refers to, for example, a portion with darkness at a level of being erroneously recognized as the lumen 42 by the lumen recognition model 92 by inputting the frame 40 to the lumen recognition model 92. The third condition is a condition in which an obstruction that obstructs the lumen 42 is shown in the frame 40 (for example, the (N+1)-th frame 40). A first example of the obstruction is a treatment tool (for example, gripping forceps, a papillotomy knife, a snare, a catheter, a guide wire, a cannula, a biopsy needle with a guide sheath, and/or a clip). A second example of the obstruction is an attachment adhering to an objective lens of the camera 52. Examples of the attachment include water droplets, residues, liquid medicines, and/or blood. The fourth condition is a condition in which the image quality triggers the erroneous specification. The image quality that triggers the erroneous specification refers to, for example, blurriness, shake, noise, and/or brightness at a level of being erroneously recognized as the lumen 42 by the lumen recognition model 92 by inputting the frame 40 to the lumen recognition model 92. The fifth condition is a condition in which a portion in the large intestine 28 (for example, a portion having a bent part) that triggers the erroneous specification is shown in the frame 40 (for example, the (N+1)-th frame 40).

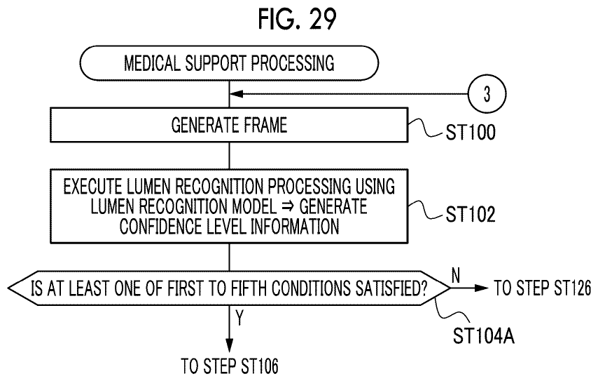

In a case in which the condition including at least one of the first to fifth conditions is used instead of the within-reliability-range condition, as an example, as shown in FIG. 29, processing of step ST104A is executed by the processor 82 instead of the processing of step ST104 included in the medical support processing. In step ST104A included in the medical support processing shown in FIG. 29, the recognition unit 82A determines whether or not at least one of the first to fifth conditions is satisfied. In step ST104A, in a case in which none of the first to fifth conditions are satisfied, a negative determination is made, and the medical support processing proceeds to step ST106 (see FIG. 28A). In step ST104A, in a case in which at least one of the first to fifth conditions is satisfied, an affirmative determination is made, and the medical support processing proceeds to step ST126 (see FIG. 28B). Therefore, in a case in which the condition including at least one of the first to fifth conditions is satisfied, the existence position of the lumen 42 is predicted by the lumen prediction processing 177 using the time-series model 94 without specifying the existence position of the lumen 42 through the lumen recognition processing 153 using the lumen recognition model 92. As a result, it is possible to prevent the information (for example, the mark 170) based on the result (for example, the lumen existence region 168) erroneously specified by the lumen recognition processing 153 using the lumen recognition model 92 from being visually recognized by the doctor 12 or the like.

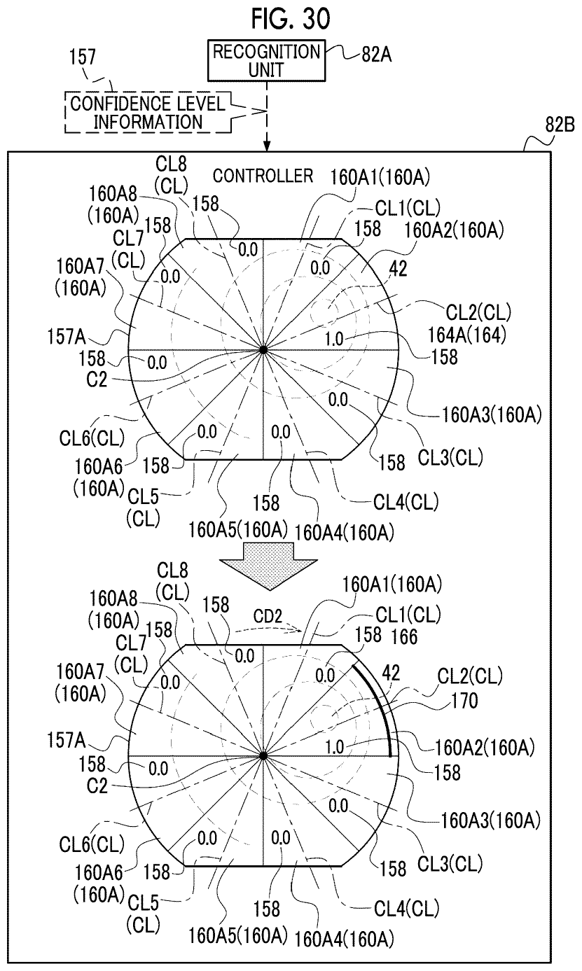

In the above-described embodiment, although the form example has been described in which the mark 170 is generated based on the vector sum 166, the present disclosure is established even in a case in which the mark 170 is not generated based on the vector sum 166. For example, as shown in FIG. 30, the mark 170 (for example, an arc-shaped mark with the center located on the center line CL) may be generated in the partition region 160A to which the maximum confidence level 158 among all the confidence levels 158 added to all the partition regions 160A of the map 157A is added, and the generated mark 170 may be displayed in the first display region 35A by the same method as in the above-described embodiment.

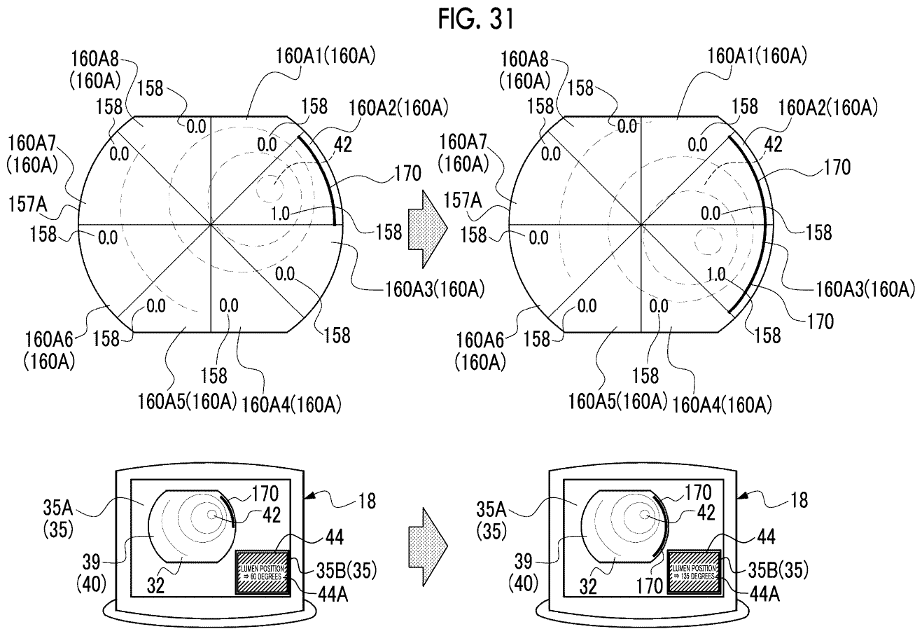

In addition, as shown in FIG. 31 as an example, in a case in which the partition region 160A to which the maximum confidence level 158 among all the confidence levels 158 added to all the partition regions 160A of the map 157A is added is changed (for example, in a case in which the partition region 160A2 is changed to the partition region 160A3), the mark 170 may be displayed at a position corresponding to the partition region 160A after the change (in the example shown in FIG. 31, the partition region 160A3), and the display of the mark 170 before the change (for example, the mark 170 displayed at the position corresponding to the partition region 160A2) may be continued. Further, as in the above-described embodiment, the display level of the mark 170 before the change may gradually decrease throughout the predetermined period.

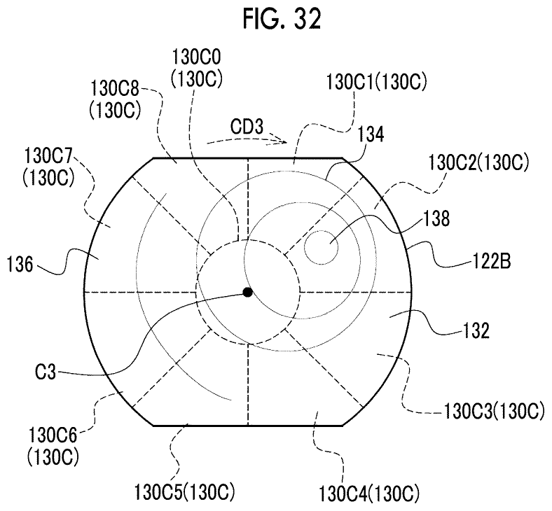

In the above-described embodiment, the example image 122A has been described, but this is merely an example, and, for example, as shown in FIG. 32, an example image 122B may be used instead of the example image 122A.

The inside of the large intestine 132 is shown in the example image 122B. In the example shown in FIG. 32, an intestinal wall 136 in which the plurality of folds 134 are formed and a lumen 138 are shown in the example image 122B.

The example image 122B is partitioned into a plurality of partition regions 130C. The plurality of partition regions 130C include a central region 130C0 and eight radial regions 130C1 to 130C8. The central region 130C0 is a circular region having a center that coincides with a center C3 of the example image 122B. The radial regions 130C1 to 130C8 are regions that exist radially from the central region 130C0 toward an outer edge of the example image 122B and are disposed along a circumferential direction CD3 of the example image 122B (in other words, around the center C3 of the example image 122B).

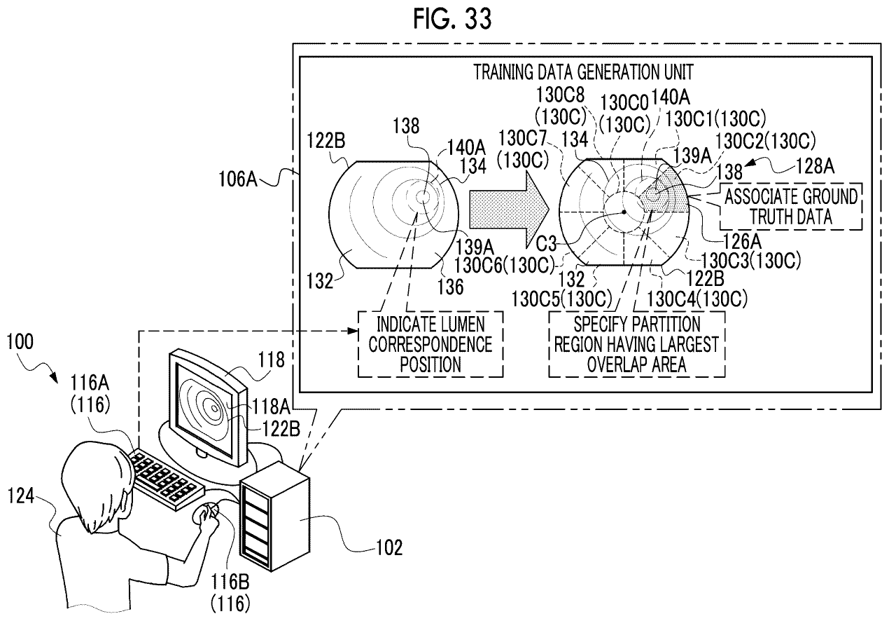

FIG. 33 is a conceptual diagram showing an example of a method of generating training data 128A by associating ground truth data 126A with the example image 122B by the training data generation unit 106A. As shown in FIG. 33, in a state in which the example image 122B is displayed on the screen 118A, the annotator 124 indicates a lumen correspondence position 139A, which is the position of the lumen 138 shown in the example image 122B, in the example image 122B, with respect to the training data generation unit 106A via the reception device 116. The training data generation unit 106A displays a circular frame 140A in a superimposed manner on the example image 122B in accordance with the instruction received by the reception device 116, and disposes the frame 140A at a position surrounding the lumen 138 shown in the example image 122B. The frame 140A is a mark that defines the lumen correspondence position 139A in the example image 122B. That is, a position of a region surrounded by the frame 140A in the example image 122B is the lumen correspondence position 139A. The size and the position of the frame 140A are freely changed on the screen 118A in response to the instruction received by the reception device 116. Here, the shape of the frame 140A is a circular shape, but may be a shape other than circular. The size of the frame 140A can be changed in response to the instruction received by the reception device 116.

The annotator 124 issues a confirmation instruction, which is an instruction to confirm the lumen correspondence position 139A, to the training data generation unit 106A via the reception device 116 in a state in which the frame 140A is disposed at the position surrounding the lumen 138. As a result, the training data generation unit 106A confirms the lumen correspondence position 139A.

The training data generation unit 106A specifies the partition region 130C having a largest area overlapping with the frame 140A that defines the lumen correspondence position 139A, from among the plurality of partition regions 130C. Then, the training data generation unit 106A generates the training data 128A by associating the ground truth data 126A with the partition region 130C in which the lumen 138 is shown, as the annotation capable of specifying the partition region 130C (in the example shown in FIG. 33, the radial region 130C2) specified from the plurality of partition regions 130C.

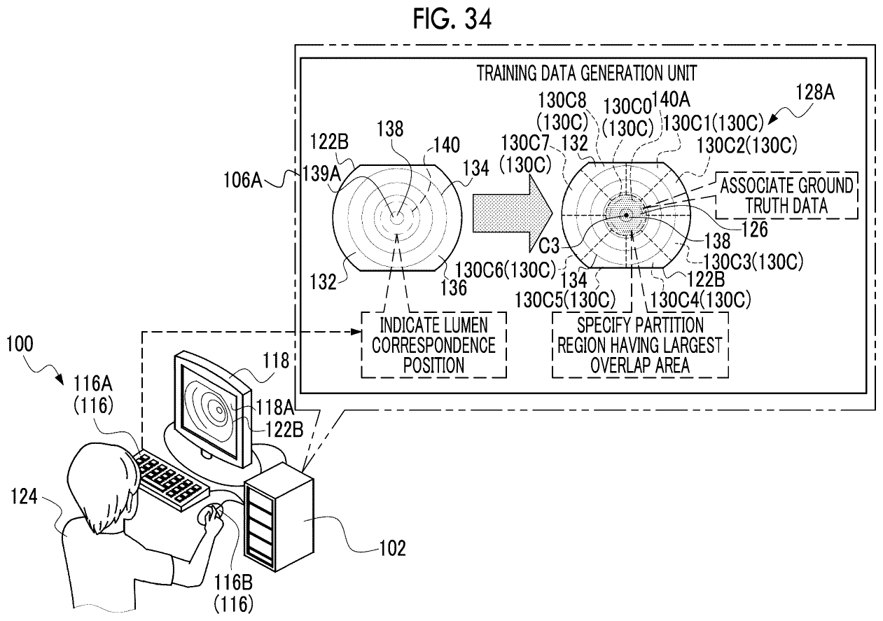

In the example shown in FIG. 33, the aspect has been shown in which the ground truth data 126 is associated with the radial region 130C2, but this is merely an example. For example, as shown in FIG. 34, in a case in which the partition region 130C having the largest area overlapping with the frame 140A among the plurality of partition regions 130C is the central region 130C0, the training data generation unit 106A generates the training data 128A by associating the ground truth data 126A with the central region 130C0.

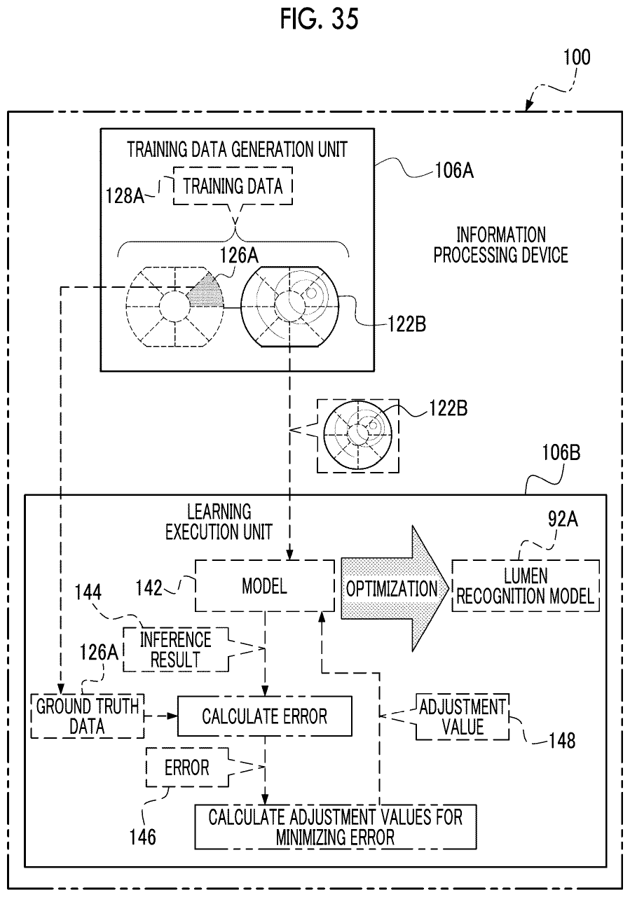

FIG. 35 is a conceptual diagram showing an example of an aspect in which the learning execution unit 106B executes machine learning using the training data 128A to generate the lumen recognition model 92. As shown in FIG. 35, in the information processing device 100, the learning execution unit 106B acquires the training data 128A generated by the training data generation unit 106A. Then, the learning execution unit 106B generates a lumen recognition model 92A by executing the machine learning using the training data 128A by the same method as in a case in which the machine learning is executed using the training data 128 in the above-described embodiment. The lumen recognition model 92A is transmitted from the information processing device 100 to the medical support device 24 via the external I/Fs 80 and 104 (see FIG. 5), and is received by the medical support device 24. Then, in the medical support device 24, the lumen recognition model 92A is stored in the storage 86 by the processor 82. The lumen recognition model 92A, which is stored in the storage 86, is used by the recognition unit 82A.

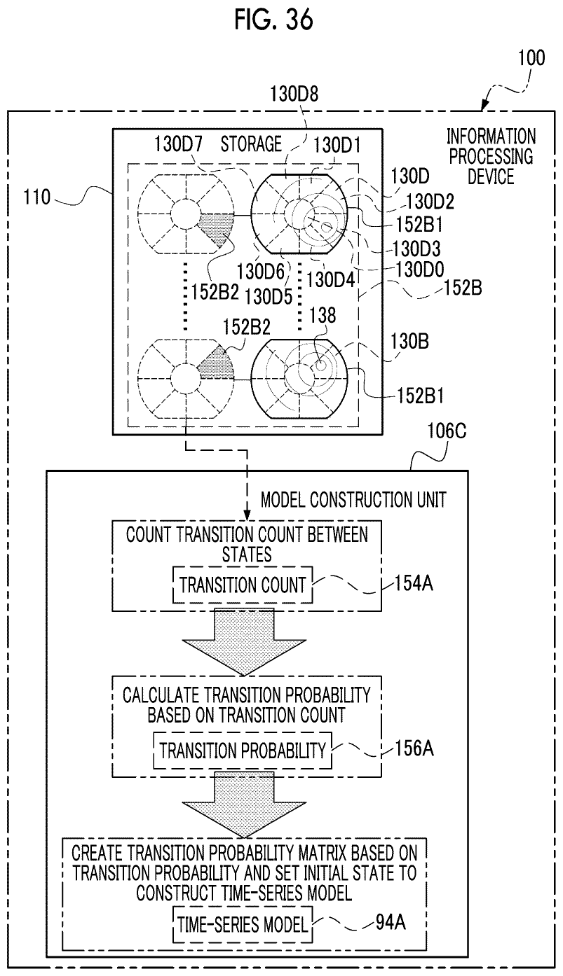

FIG. 36 is a conceptual diagram showing an example of processing contents in which the Markov model based on the Markov chain is constructed as the time-series model 94A by the model construction unit 106C. As shown in FIG. 36, a time-series dataset 152B includes a plurality of time-series images 152B1 arranged in time series. As in the plurality of time-series images 152A1 described in the above-described embodiment, the plurality of time-series images 152B1 are a plurality of images corresponding to the plurality of frames 40 arranged in time series and included in the endoscopic video image 39 (that is, the plurality of images representing the plurality of frames 40 arranged in time series).

As in the example image 122B, the inside of the large intestine 132 is shown in the time-series image 152B1. In addition, the time-series image 152B1 is partitioned into a plurality of partition regions 130D by the same method as in the example image 122B. In the example shown in FIG. 36, as the plurality of partition regions 130D, partition regions 130D0 to 130D8 are shown. Geometrical characteristics of all the partition regions 130D in the time-series image 152A1 correspond to geometrical characteristics of all the partition regions 130C in the example image 122B. In addition, by the same method as in a case in which the ground truth data 126A is associated with each example image 122B, the ground truth data 152B2 corresponding to the ground truth data 126A is also associated with each time-series image 152B1.

The model construction unit 106C acquires, in time series, a plurality of pieces of ground truth data 152B2 associated with the plurality of time-series images 152B1 arranged in time series, and counts a transition count 154A between the respective states in which the lumen 138 is shown in the partition region 130D in the plurality of time-series images 152B1 arranged in time series from the plurality of pieces of ground truth data 152B2 arranged in time series. Each state in which the lumen 138 is shown in the partition region 130D is specified from the ground truth data 152B2 associated with the plurality of time-series images 152B1.

Examples of the state in which the lumen 138 is shown in the partition region 130D include nine states, that is, seventeenth to twenty-fifth states. The seventeenth state means a state in which the lumen 138 is shown in the partition region 130D0. The eighteenth state means a state in which the lumen 138 is shown in the partition region 130D1. The nineteenth state means a state in which the lumen 138 is shown in the partition region 130D2. The twentieth state means a state in which the lumen 138 is shown in the partition region 130D3. The twenty-first state means a state in which the lumen 138 is shown in the partition region 130D4. The twenty-second state means a state in which the lumen 138 is shown in the partition region 130D5. The twenty-third state means a state in which the lumen 138 is shown in the partition region 130D6. The twenty-fourth state means a state in which the lumen 138 is shown in the partition region 130D7. The twenty-fifth state means a state in which the lumen 138 is shown in the partition region 130D8.

The transition count 154A means the number of times of transition among the seventeenth to twenty-fifth states (in other words, the number of times the partition region 130D in which the lumen 138 is shown transitions from one partition region 130D to another partition region 130D among the nine partition regions 130D to 130D8).

The model construction unit 106C counts the transition count 154A by pairing a state in an N-th time-series image 152B1 and a state in an (N+1)-th time-series image 152B1, and totals the transition count 154A among the seventeenth to twenty-fifth states. That is, the model construction unit 106C totals the number of times of transition from a certain state among the seventeenth to twenty-fifth states to a next state, as the transition count 154A.

The model construction unit 106C calculates a transition probability 156A based on the transition count 154A. The transition probability 156A means a probability indicating the transition from one state among the seventeenth to twenty-fifth states to another state among the seventeenth to twenty-fifth states. For example, in a case in which each of N and M is any natural number of 17 to 25, the transition probability 156A from an N-th state to an M-th state is a value obtained by dividing the transition count 154A from the N-th state to the M-th state by the total transition count from the N-th state.

The model construction unit 106C constructs the time-series model 94A by creating a transition probability matrix based on the transition probability 156A and setting an initial state. In a case in which there are nine states, that is, the seventeenth to twenty-fifth states, the transition probability matrix created based on the transition probability 156A is a 9×9 matrix. The transition probability matrix is a core of the time-series model 94A. The initial state means an initial state (for example, one state selected from the seventeenth to twenty-fifth states based on the plurality of time-series images 152B1) for starting the prediction via the time-series model 94A. Here, although the nine states, that is, the seventeenth to twenty-fifth states, are described, these are merely examples, and there may be fewer than nine states or more than nine states.

The time-series model 94A constructed by the model construction unit 106C is transmitted from the information processing device 100 to the medical support device 24 via the external I/Fs 80 and 104 (see FIG. 11), and is received by the medical support device 24. In the medical support device 24, the time-series model 94A is stored in the storage 86 by the processor 82. The time-series model 94A, which is stored in the storage 86, is used by the recognition unit 82A.

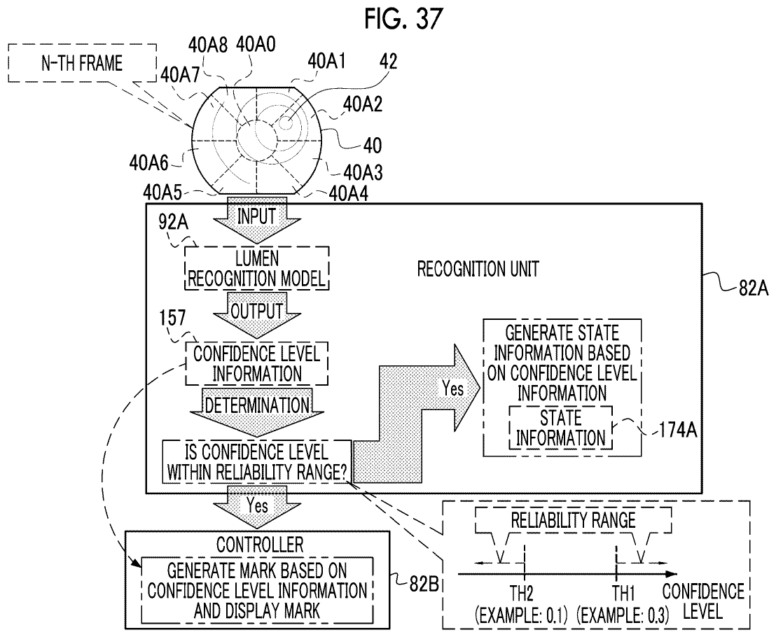

FIGS. 37 to 40 show an example of processing contents corresponding to the examples shown in FIGS. 17, 20, 23, and 25. As shown in FIGS. 37 to 40, the frame 40 includes partition regions 40A0 to 40A8 corresponding to the partition regions 130D0 to 130D8. Geometrical characteristics of the partition regions 40A0 to 40A8, which are applied to the frame 40, in the frame 40 are the same as geometrical characteristics of the partition regions 130D0 to 130D8, which are applied to the time-series image 152B1, in the time-series image 152B1.

As shown in FIG. 37, in a case in which the N-th frame 40 is input to the lumen recognition model 92A, confidence level information 157 is output from the lumen recognition model 92A. As in the confidence level information 157 described in the above-described embodiment, the confidence level information 157 includes a map corresponding to the map 157A described in the above-described embodiment. The map included in the confidence level information 157 has nine partition regions corresponding to the partition regions 40A0 to 40A8, and the confidence level 158 is added to each partition region of the map included in the confidence level information 157 by the same method as in the above-described embodiment. In the example shown in FIG. 37, in a case in which the within-reliability-range condition for all the confidence levels 158 added to each of the partition regions of the map included in the confidence level information 157 is satisfied, the controller 82B generates the mark 170 based on the confidence level information 157 output from the lumen recognition model 92A by inputting the N-th frame 40 to the lumen recognition model 92A and displays the generated mark 170 in the first display region 35A, by the same method as in the above-described embodiment.

In addition, in a case in which the within-reliability-range condition for all the confidence levels 158 added to each of the partition regions of the map included in the confidence level information 157 is satisfied, the controller 82B generates the state information 174A corresponding to the state information 174 described in the above-described embodiment based on the confidence level information 157 by the same method as in in the above-described embodiment.

The state information 174A means information capable of specifying a state in which the lumen 42 is shown in the partition region of the map included in the confidence level information 157. Examples of the state in which the lumen 42 is shown in the partition region of the map included in the confidence level information 157 include nine states, that is, twenty-sixth to thirty-fourth states. Therefore, the state information 174A can also be referred to as information capable of specifying which of the twenty-sixth to thirty-fourth states corresponds to the state in which the lumen 42 is shown in the partition region of the map included in the confidence level information 157.

The twenty-sixth to thirty-fourth states are states corresponding to the seventeenth to twenty-fifth states described above. The twenty-sixth state means a state in which the lumen 42 is shown in the partition region corresponding to the partition region 40A0 among the nine partition regions of the map included in the confidence level information 157. The twenty-seventh state means a state in which the lumen 42 is shown in the partition region corresponding to the partition region 40A1 among the nine partition regions of the map included in the confidence level information 157. The twenty-eighth state means a state in which the lumen 42 is shown in the partition region corresponding to the partition region 40A2 among the nine partition regions of the map included in the confidence level information 157. The twenty-ninth state means a state in which the lumen 42 is shown in the partition region corresponding to the partition region 40A3 of the nine partition regions of the map included in the confidence level information 157. The thirtieth state means a state in which the lumen 42 is shown in the partition region corresponding to the partition region 40A4 of the nine partition regions of the map included in the confidence level information 157. The thirty-first state means a state in which the lumen 42 is shown in the partition region corresponding to the partition region 40A5 among the nine partition regions of the map included in the confidence level information 157. The thirty-second state means a state in which the lumen 42 is shown in the partition region corresponding to the partition region 40A6 among the nine partition regions of the map included in the confidence level information 157. The thirty-third state means a state in which the lumen 42 is shown in the partition region corresponding to the partition region 40A7 among the nine partition regions of the map included in the confidence level information 157. The thirty-fourth state means a state in which the lumen 42 is shown in the partition region corresponding to the partition region 40A8 among the nine partition regions of the map included in the confidence level information 157.

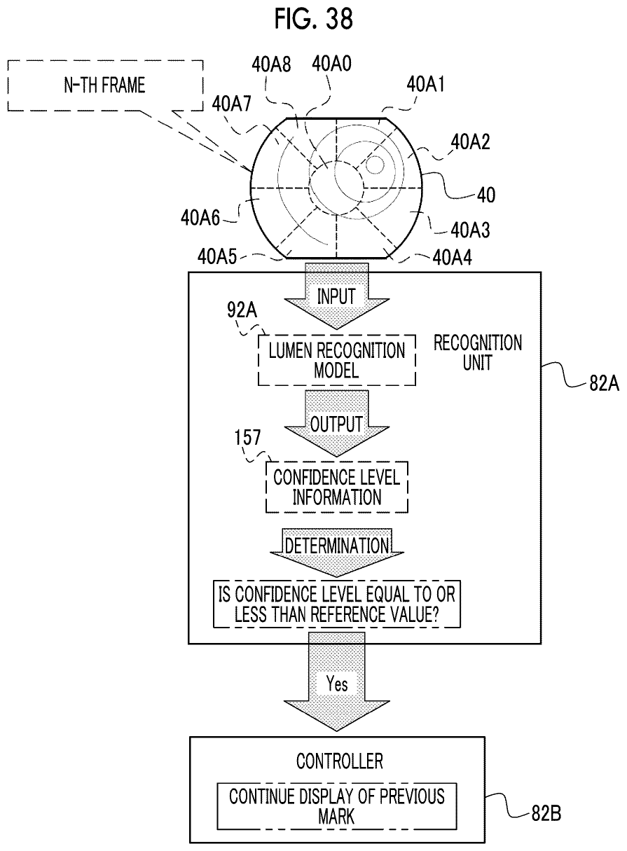

As shown in FIG. 38, in a case in which all the confidence levels 158 included in the confidence level information 157 output from the lumen recognition model 92A by inputting the N-th frame 40 to the lumen recognition model 92A are equal to or less than the reference value, the controller 82B continues the display of the previous mark 170 by the same method as in the above-described embodiment.

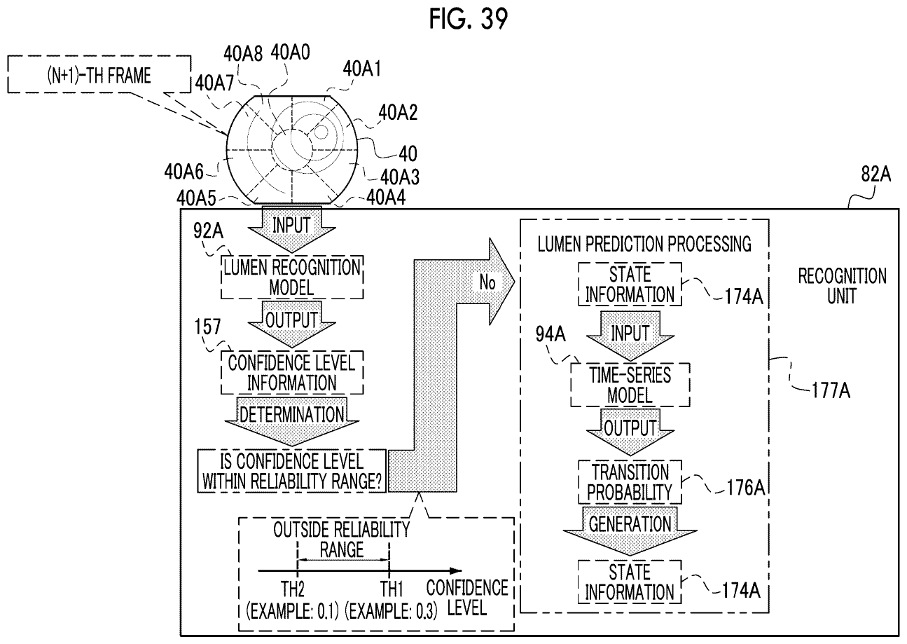

As shown in FIG. 39, in a case in which the outside-reliability-range condition for all the confidence levels 158 included in the confidence level information 157 output from the lumen recognition model 92A by inputting the (N+1)-th frame 40 to the lumen recognition model 92A is satisfied, the recognition unit 82A executes the lumen prediction processing 177A using the time-series model 94A by the same method as in the lumen prediction processing 177 described in the above-described embodiment. In the lumen prediction processing 177A, latest state information 174A is input to the time-series model 94A. That is, the recognition unit 82A inputs the latest state information 174A to the time-series model 94 to cause the time-series model 94A to output a transition probability 176A. Here, examples of the latest state information 174A include the state information 174A (see FIG. 37) generated in a case in which the processing shown in FIG. 37 is performed on the preceding frame 40, or the state information 174A (see FIG. 39) generated in a case in which the lumen prediction processing 177A shown in FIG. 39 is performed on the preceding frame 40. The time-series model 94A outputs the transition probability 176A corresponding to the transition probability 176 described in the above-described embodiment in response to the input of the latest state information 174. Here, although the form example has been described in which the time-series model 94A outputs the transition probability 176A in response to the input of one piece of state information 174A, this is merely an example. For example, in a case in which the Markov model based on the N-th order Markov chain is used instead of the time-series model 94A, the Markov model may output the transition probability 176A in response to the input of a plurality of pieces of state information 174A that were previously obtained.

The transition probability 176A output from the time-series model 94A is a probability of transition from the state specified from the state information 174A input to the time-series model 94A to the next state. In the lumen prediction processing 177A, the state information 174A is generated based on the transition probability 176A output from the time-series model 94A. Here, the state specified from the generated state information 174A is a next state (that is, a state in which the lumen 42 is shown in any one of the partition regions 40A0 to 40A8 used for the (N+1)-th frame 40) that transitions from the state specified from the state information 174A input to the time-series model 94A. The next state that transitions from the state specified from the state information 174A input to the time-series model 94A is any one of the twenty-sixth to thirty-fourth states.

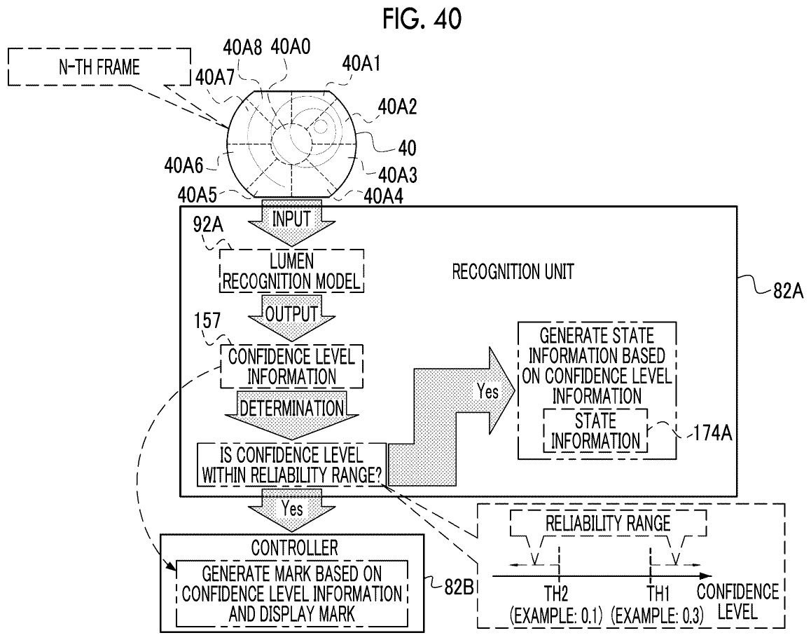

As shown in FIG. 40, in a case in which the within-reliability-range condition for each of the confidence levels 158 added to the partition regions of the map included in the confidence level information 157 output from the lumen recognition model 92A by inputting the (N+1)-th frame 40 to the lumen recognition model 92A is satisfied, the recognition unit 82A and the controller 82B perform the same processing as in the example shown in FIG. 37. In this case, the controller 82B generates the mark 170 based on the confidence level information 157 output from the lumen recognition model 92A by inputting the (N+1)-th frame 40 to the lumen recognition model 92A. Then, the controller 82B displays the mark 170 in the first display region 35A by the same method as in the above-described embodiment. The mark 170 generated based on the confidence level information 157 output from the lumen recognition model 92A by inputting the (N+1)-th frame 40 to the lumen recognition model 92A is information capable of specifying the existence position of the lumen 42, which is shown in the (n+1)-th frame 40, in the (n+1)-th frame 40.

In this way, in a case in which the within-reliability-range condition for each of the confidence levels 158 added to the partition regions of the map included in the confidence level information 157 output from the lumen recognition model 92A by inputting the (N+1)-th frame 40 to the lumen recognition model 92A is satisfied, the controller 82B preferentially displays, on the screen 35, the information based on the processing result obtained by the lumen recognition model 92A (that is, the mark 170 generated based on the confidence level information 157) over the information based on the processing result obtained by the time-series model 94A (that is, the mark 170 generated based on the state information 174A generated by the lumen prediction processing 177A). As a result, the doctor 12 or the like can accurately ascertain the existence position of the lumen 42 shown in the (N+1)-th frame 40.

In the above-described embodiment, the arc has been described as the shape of the mark 170, but this is merely an example, and the mark having another shape, such as a toroidal mark or a polygonal mark, may be used.

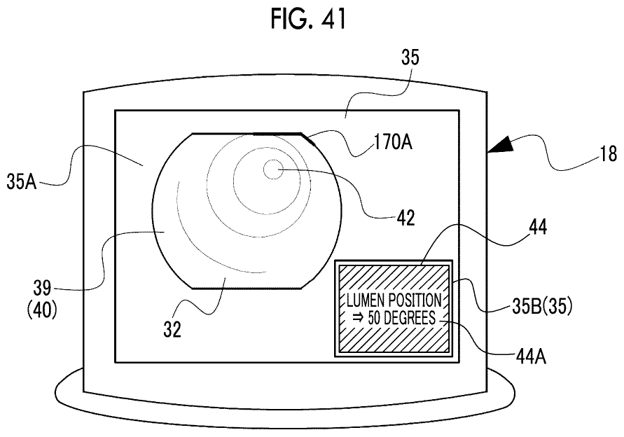

As shown in FIG. 41 as an example, a mark 170A having a shape along an outer edge of the frame 40 may be generated and displayed in the first display region 35A instead of the mark 170. Examples of the method of generating the mark 170A include a method in which the mark 170 is projected on the outer edge of the map 157A (see FIG. 16) from the center C2 (see FIG. 16) side to generate the mark having a shape along the outer edge of the map 157A as the mark 170A. The frame 40 input to the lumen recognition model 92 for generating the confidence level information 157 including the map 157A is displayed in the first display region 35A, and the mark 170A is displayed in accordance with the display timing of the frame 40.

Here, since the mark 170 is formed inside the map 157A, as an example of the method for generating the mark 170A, the form example has been described in which the mark 170 is projected to the outer edge of the map 157A from the center C2 side, but, in a case in which the mark 170 is formed outside the map 157A (for example, in a case in which the mark 170 is formed below an upper end of the map 157A or below a lower end of the map 157A), the mark 170 need only be projected to the outer edge of the map 157A from the outside of the map 157A toward the center C2 side.

As described above, in a case in which the mark having a shape along the outer edge of the frame 40 is displayed in the first display region 35A, the display of the object that visually obstructs the frame 40 in the first display region 35A is suppressed, so that it is possible to allow the doctor 12 to accurately ascertain the position of the lumen 42, which is shown in the frame 40, in the frame 40 while ensuring the visibility of the frame 40 displayed in the first display region 35A.

Further, the mark 170A may also be updated in accordance with the display timing of the frame 40 by the same method in which the mark 170 is updated in accordance with the display timing of the frame 40. In this way, the doctor 12 can visually recognize the mark 170A in accordance with the contents of the frame 40 displayed in the first display region 35A.

In the above-described embodiment, the form example has been described in which the mark 170 is displayed in a superimposed manner on the frame 40, but this is merely an example, and the mark 170 may be displayed outside the frame 40. In a case in which the mark 170 is displayed outside the frame 40, there is no object that visually obstructs the frame 40 displayed in the first display region 35A, and thus the visibility of the frame 40 displayed in the first display region 35A can be improved.

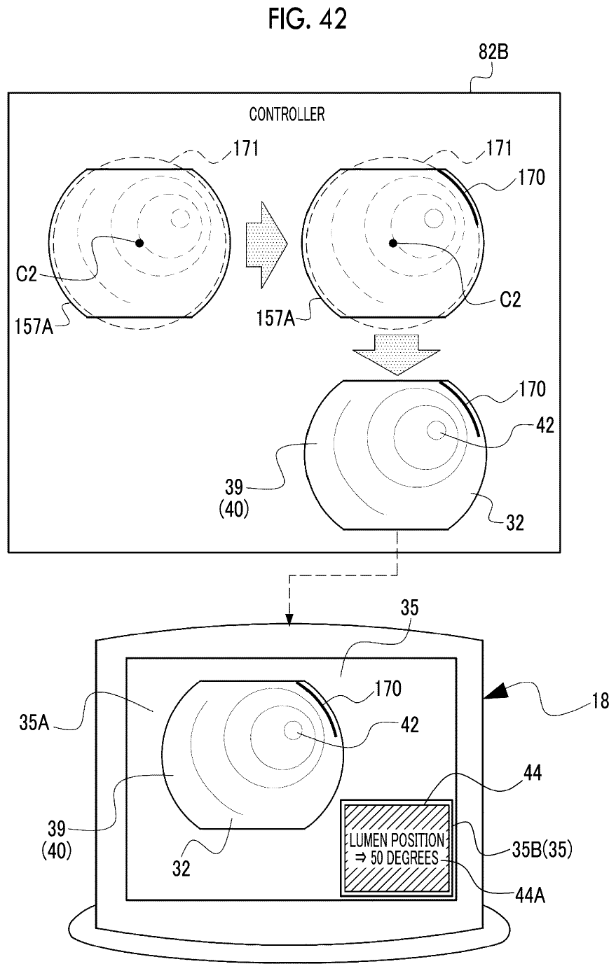

In the above-described embodiment, the form example has been described in which the controller 82B generates the arc-shaped mark 170 capable of specifying the range from the one end to the other end of the lumen existence region 168 (see FIG. 16) in the circumferential direction CD2 (see FIG. 16), and displays the generated mark 170 in the first display region 35A, but this is merely an example. For example, as shown in FIG. 42, the controller 82B may display the mark 170 in the first display region 35A by displaying, in the first display region 35A, at least one marker 171 capable of specifying the range from the one end to the other end of the lumen existence region 168 (see FIG. 16) in the circumferential direction CD2 (see FIG. 16) among a plurality of hidden markers 171 associated with the map 157A. For example, the number of the plurality of markers 171 (in other words, the number of divided parts) may be “40” (=8×5) obtained by equally partitioning each of the eight partition regions 160A (see FIGS. 14 to 16) into five parts or “80” (=8×10) obtained by equally partitioning each of the eight partition regions 160A into ten parts. The number of the plurality of markers 171 may be a number other than these numbers. As the number of the markers 171 increases, the resolution of the display of the mark 170 increases. That is, the controller 82B can generate and display the mark 170 in more detail as the number of the markers 171 increases.

In the example shown in FIG. 42, a plurality of arc-shaped markers disposed at constant intervals along a circle having a center that coincides with the center C2 of the map 157A are shown as examples of the plurality of hidden markers 171.

In the example shown in FIG. 42, the display of the mark 170 in the first display region 35A is carried out by displaying at least one marker 171 capable of specifying the range from the one end to the other end of the lumen existence region 168 (see FIG. 16) in the circumferential direction CD2 (see FIG. 16).

In the example shown in FIG. 42, the mark 170 is displayed in a superimposed manner on the frame 40 displayed in the first display region 35A, but the mark 170 may be displayed outside the frame 40 depending on the position of the lumen existence region 168 (see FIG. 16). For example, at least one marker 171 at a position (in the example shown in FIG. 20, above the upper end of the map 157A in a front view and below the lower end of the map 157A in a front view) away from the map 157A is displayed, and thus the mark 170 is displayed outside the frame 40 displayed in the first display region 35A.

In the example shown in FIG. 42, the plurality of arc-shaped markers disposed at constant intervals along a circle having a center that coincides with the center C2 of the map 157A have been shown as examples of the plurality of hidden markers 171, but this is merely an example. For example, the plurality of hidden markers 171 may be a plurality of markers disposed at constant intervals along the outer edge of the map 157A.

In the example shown in FIG. 42, the plurality of arc-shaped markers that are disposed at constant intervals along a circle that has a center coinciding with the center C2 of the map 157A and a part of which overlaps the map 157A have been shown, but this is merely an example. For example, a plurality of arc-shaped markers that are disposed at constant intervals along a circle that has a center coinciding with the center C2 of the map 157A and surrounds the map 157A may be used. In this case, the mark 170 is displayed outside the frame 40 by displaying at least one marker 171 capable of specifying the range from the one end to the other end of the lumen existence region 168 (see FIG. 16) in the circumferential direction CD2 (see FIG. 16).

As described above, the display of the mark 170 is carried out by displaying at least one marker 171 at a position corresponding to the position of the lumen existence region 168 (see FIG. 16) among the plurality of hidden markers 171, so that a processing load required for the display of the mark 170 can be reduced.

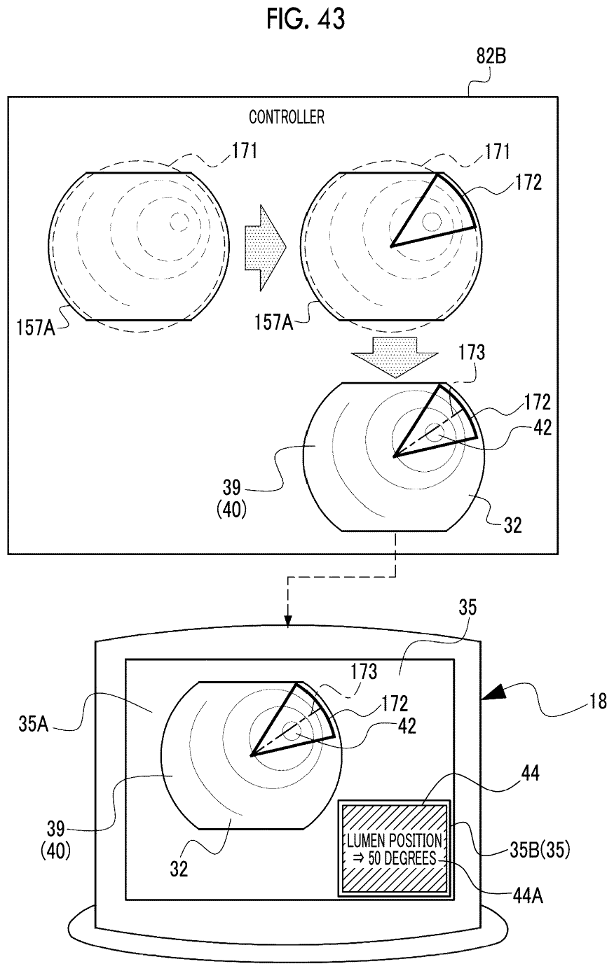

In the example shown in FIG. 42, the form example has been described in which the mark 170 is displayed, but this is merely an example, and, for example, as shown in FIG. 43, the outer contour line 172 may be displayed in the first display region 35A instead of the mark 170. The outer contour line 172 is a line that outlines an outer contour of the lumen existence region 168 (see FIG. 16). In the example shown in FIG. 43, an outer contour line 172 is displayed in a superimposed manner on the frame 40 displayed in the first display region 35A. In a case in which the outer contour line 172 is displayed in a superimposed manner on the frame 40 in this way, the doctor 12 can visually recognize that the lumen 42 is shown within the outer contour line 172.

In addition, as shown in FIG. 43 as an example, a line segment 173 (for example, a line segment extending from the center C2 along the vector sum 166) along the vector sum 166 may be displayed in a superimposed manner on the frame 40. In this case, the doctor 12 can visually recognize that the lumen 42 is shown on the line segment 173 displayed in a superimposed manner on the frame 40.

In the above-described embodiment, eight partition regions 160A are shown as examples, but the number of the partition regions 160A may be fewer than eight or may be nine or more. Further, the number of the partition regions 130A need only also be determined in accordance with the number of the partition regions 160A.

In the above-described embodiment, the form example has been described in which the frame 40 is displayed on the screen 35 and the mark 170 is displayed in a state of being comparable with the frame 40 (for example, the form example in which the mark 170 is displayed in a superimposed manner on the frame 40), but this is merely an example. For example, the map 157A may be displayed on the screen 35, and the mark 170 may be displayed in a state of being comparable with the map 157A. One example of the comparable display example is displaying the mark 170 in a superimposed manner on the map 157A.

In the above-described embodiment, the form example has been described in which the visible information 44A is displayed in the second display region 35B, but this is merely an example. For example, audible information (for example, an electronic sound or a language sound) capable of specifying the position of the lumen 42 in the frame 40 shown in the frame 40 may be output from a speaker (not shown). In addition, the information in which the frame 40 and the mark 170 or the like are combined and/or the visible information 44A may be printed on a medium by a printer. In addition, the information in which the frame 40 and the mark 170 or the like are combined, the visible information 44A, and/or the above-described audible information may be stored in a storage medium (for example, a storage provided in an external device, such as the storage 76, the storage 86, or the server).

In the above-described embodiment, the endoscopic image has been described as the frame 40, but this is merely an example, and the present disclosure is established even in a case in which a medical image such as an MRI image, a CT image, an X-ray image, or three-dimensional volume data (for example, three-dimensional volume data generated based on a plurality of slice images such as a plurality of MRI images or a plurality of CT images) is

applied instead of the frame 40.

In the above-described embodiment, although the form example has been described in which the frame 40 in which the lumen 42 is shown is input to the lumen recognition model 92 or 92A, the present disclosure is not limited to this. For example, even in a case in which the frame 40 in which the lumen 42 is not shown is input to the lumen recognition model 92 or 92A, as long as the frame 40 that satisfies the erroneous specification triggering condition is input to the lumen recognition model 92 or 92A, the erroneous specification (in other words, the erroneous recognition) of the lumen 42 is suppressed, so that the present disclosure is established.

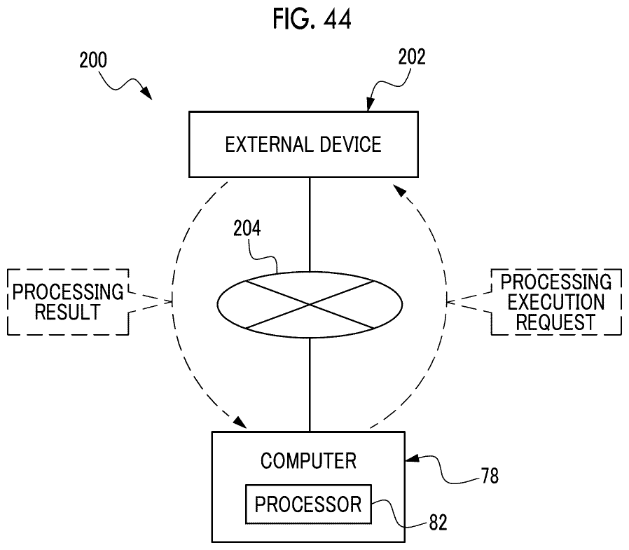

In the above-described embodiment, the form example has been described in which the medical support processing is executed by the computer 78, but the present disclosure is not limited to this, and at least a part of the medical support processing may be executed by a device provided outside the computer 78. Hereinafter, an example of this case will be described with reference to FIG. 44.

FIG. 44 is a conceptual diagram showing an example of a configuration of an endoscope system 200. The endoscope system 200 is an example of an “endoscope system” according to the present disclosure. The endoscope system 200 is different from the endoscope system 10 according to the above-described embodiment in that an external device 202 is provided.

The external device 202 is connected communicably to the computer 78 via a network 204 (for example, a WAN and/or a LAN).

Examples of the external device 202 include at least one server that directly or indirectly transmits and receives data to and from the computer 78 via the network 204. The external device 202 receives a processing execution instruction issued from the processor 82 of the computer 78 via the network 204. Then, the external device 202 executes processing corresponding to the received processing execution instruction, and transmits a processing result to the computer 78 via the network 204. In the computer 78, the processor 82 receives the processing result transmitted from the external device 202 via the network 204, and executes processing using the received processing result.

Examples of the processing execution instruction include an instruction for the external device 202 to execute at least a part of the medical support processing. A first example of the at least a part of the medical support processing (that is, processing to be executed by the external device 202) is the lumen recognition processing 153. In this case, the external device 202 executes the lumen recognition processing 153 in response to the processing execution instruction issued from the processor 82 via the network 204, and transmits the confidence level information 157 to the computer 78 via the network 204. In the computer 78, the processor 82 receives the confidence level information 157, and executes the same processing as in the above-described embodiment by using the received confidence level information 157.

A second example of the at least a part of the medical support processing (that is, processing to be executed by the external device 202) is the lumen prediction processing 177. In this case, the external device 202 executes the lumen prediction processing 177 in response to the processing execution instruction issued from the processor 82 via the network 204, and transmits the transition probability 176 and/or the state information 174 to the computer 78 via the network 204. In the computer 78, the processor 82 receives the transition probability 176 and/or the state information 174, and executes the same processing as in the above-described embodiment by using the received transition probability 176 and/or state information 174.

A third example of the at least a part of the medical support processing (that is, the processing to be executed on the external device 202) is processing performed by the controller 82B (for example, processing related to display). In this case, the external device 202 executes the processing by the controller 82B in response to the processing execution instruction issued from the processor 82 via the network 204, and transmits the processing result (for example, the frame 40 and/or the mark 170) to the computer 78 via the network 204. In the computer 78, the processor 82 receives the processing result and executes the same processing (for example, the display using the display device 18) as the processing in the above-described embodiment using the received processing result.

A fourth example of the at least a part of the medical support processing (that is, the processing to be executed by the external device 202) includes various types of determination processing (for example, the determination of whether or not the within-reliability-range condition is satisfied, the determination of whether or not the confidence level 158 is equal to or less than the reference value, and/or the determination of whether or not the outside-reliability-range condition is satisfied). In this case, the external device 202 executes the processing related to various determinations in accordance with the processing execution instruction issued from the processor 82 via the network 204, and transmits the processing result (for example, the determination result of whether or not the within-reliability-range condition is satisfied, the determination result of whether or not the confidence level 158 is equal to or less than the reference value, and/or the determination result of whether or not the outside-reliability-range condition is satisfied) to the computer 78 via the network 204. In the computer 78, the processor 82 receives the processing result and executes the same processing as the processing in the above-described embodiment using the received processing result.

The external device 202 may be carried out by cloud computing. The cloud computing is merely an example, and the external device 202 may be carried out by network computing, such as fog computing, edge computing, or grid computing.

In the above-described embodiment, the form example has been described in which the medical support program 90 is stored in the storage 86, but the present disclosure is not limited to this. For example, the medical support program 90 may be stored in a portable non-transitory computer-readable storage medium such as an SSD or a USB memory. The medical support program 90, which is stored in the non-transitory storage medium, is installed in the computer 78 of the endoscope system 10. The processor 82 executes the medical support processing in accordance with the medical support program 90.

Further, the medical support program 90 may be stored in a storage device of another computer, a server, or the like that is connected to the endoscope system 10 via the network, and the medical support program 90 may be downloaded and installed in the computer 78 in response to a request from the endoscope system 10.

It is not necessary to store the entire medical support program 90 in a storage device of another computer or a server device connected to the endoscope system 10 or to store the entire medical support program 90 in the storage 86, and a part of the medical support program 90 may be stored.

The following various processors can be used as hardware resources for executing the medical support processing. An example of the processor is a CPU that is a general-purpose processor that executes software, that is, a program, to function as the hardware resource for executing the medical support processing. Another example of the processor is a dedicated electric circuit that is a processor having a dedicated circuit configuration designed to execute specific processing, such as an FPGA, a PLD, or an ASIC. All processors have a memory built therein or connected thereto, and all processors use the memory to execute the medical support processing.

The hardware resource for executing the medical support processing may be configured by one of the various processors or by a combination of two or more processors of the same type or different types (for example, a combination of a plurality of FPGAs or a combination of a CPU and an FPGA). Furthermore, the hardware resource for executing the medical support processing may be one processor.

A first example of the configuration in which the hardware resource is configured by one processor is an aspect in which one processor is configured by a combination of one or more CPUs and software, and this processor functions as the hardware resource for executing the medical support processing. As a second example, as typified by an SoC or the like, there is a form in which a processor that implements all functions of a system including a plurality of hardware resources executing the medical support processing with one IC chip is used. In this way, the medical support processing is carried out by using one or more of the various processors as the hardware resource.

Furthermore, as the hardware structure of the various processors, specifically, an electronic circuit in which circuit elements, such as semiconductor elements, are combined can be used. The above-described medical support processing is merely an example. Therefore, it goes without saying that unnecessary steps may be deleted, new steps may be added, or the processing order may be changed, within a range that does not deviate from the gist of the present disclosure.

The above-described contents and the above-shown contents are the detailed description of the parts according to the present disclosure, and are merely examples of the present disclosure. For example, the descriptions of the configurations, the functions, the operations, and the effects are the descriptions of the examples of the configurations, the functions, the operations, and the effects of the parts according to the present disclosure. Therefore, it goes without saying that unnecessary parts may be deleted, new elements may be added, or replacements may be made with respect to the above-described contents and the above-shown contents within a range that does not deviate from the gist of the present disclosure. In order to avoid confusion and to facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which each of the documents, the patent applications, and the technical standards are specifically and individually stated to be described by reference.

In regard to the above-described embodiment, the following supplementary notes will be further disclosed.

### Supplementary Note 1

A medical support device comprising: a processor configured to: acquire a medical image generated by imaging an inside of a luminal organ; and selectively output a plurality of pieces of lumen specification information that are information capable of specifying an existence position of a lumen, which is shown in the medical image, in the medical image, in which the medical image is classified into a first medical image and a second medical image obtained later than the first medical image, the plurality of pieces of lumen specification information include first lumen specification information and second lumen specification information, the first lumen specification information is generated based on information obtained from a trained model in a case in which the first medical image is input to the trained model, and is information capable of specifying a first existence position that is the existence position of the lumen, which is shown in the first medical image, in the first medical image, the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image, the first medical image or an image corresponding to the first medical image has a plurality of partition regions obtained by partitioning the first medical image or the image corresponding to the first medical image along a circumferential direction, the trained model generates a plurality of confidence levels corresponding to the plurality of partition regions and indicating that the lumen is shown for each of the plurality of partition regions, in a case in which the first medical image or the image corresponding to the first medical image is input to the trained model, the first lumen specification information is information capable of specifying the first existence position more precisely than the partition regions in the first medical image or the image corresponding to the first medical image, the trained model generates the first lumen specification information based on the plurality of partition regions and the plurality of confidence levels, and the first lumen specification information includes a mark capable of specifying a region determined as the lumen existence region based on a second vector in the first medical image or the image corresponding to the first medical image.

### Supplementary Note 2

The medical support device according to supplementary note 1, in which a shape of the mark is an arc, and a center of the arc is a center of the medical image or the image corresponding to the medical image.

### Supplementary Note 3

The medical support device according to supplementary note 2, in which a shape of the mark is a shape along an outer edge of the medical image or an outer edge of the image corresponding to the medical image.

### Supplementary Note 4

The medical support device according to any one of supplementary notes 1 to 3, in which a plurality of markers that are hidden are associated with the medical image or the image corresponding to the medical image, the medical image or the image corresponding to the medical image is displayed on a screen, and the mark is displayed on the screen by displaying at least one marker corresponding to a position of the lumen existence region among the plurality of markers.

### Supplementary Note 5

A medical support device comprising: a processor configured to: acquire a medical image generated by imaging an inside of a luminal organ; and selectively output a plurality of pieces of lumen specification information that are information capable of specifying an existence position of a lumen, which is shown in the medical image, in the medical image, in which the medical image is classified into a first medical image and a second medical image obtained later than the first medical image, the plurality of pieces of lumen specification information include first lumen specification information and second lumen specification information, the first lumen specification information is generated based on information obtained from a trained model in a case in which the first medical image is input to the trained model, and is information capable of specifying a first existence position that is the existence position of the lumen, which is shown in the first medical image, in the first medical image, the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image, the second medical image or an image corresponding to the second medical image has a plurality of partition regions obtained by partitioning the second medical image or the image corresponding to the second medical image along a circumferential direction, the information obtained from the time-series model is a plurality of transition probabilities, the second lumen specification information is information capable of specifying the second existence position more precisely than the partition regions in the second medical image or the image corresponding to the second medical image, and the second lumen specification information is generated based on the plurality of partition regions and the plurality of transition probabilities.

What is claimed is:

1. A medical support device comprising:
a processor configured to:
acquire a medical image generated by imaging an inside of a luminal organ; and
selectively output a plurality of pieces of lumen specification information that are information capable of specifying an existence position of a lumen, which is shown in the medical image, in the medical image,
wherein the medical image is classified into a first medical image and a second medical image obtained later than the first medical image,
the plurality of pieces of lumen specification information include first lumen specification information and second lumen specification information,
the first lumen specification information is generated based on information obtained from a trained model in a case in which the first medical image is input to the trained model, and is information capable of specifying a first existence position that is the existence position of the lumen, which is shown in the first medical image, in the first medical image, and
the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image.

2. The medical support device according to claim 1,
wherein the processor is configured to output the second lumen specification information in a case in which the second medical image is an image that satisfies an erroneous specification triggering condition that triggers erroneous specification of the second existence position by the trained model.

3. The medical support device according to claim 2,
wherein the plurality of pieces of lumen specification information include third lumen specification information,
the processor is configured to output the third lumen specification information in a case in which the second medical image is an image that does not satisfy the erroneous specification triggering condition, and
the third lumen specification information is generated based on information obtained from the trained model in a case in which the second medical image is input to the trained model, and is information capable of specifying a third existence position that is the existence position of the lumen, which is shown in the second medical image, in the second medical image.

4. The medical support device according to claim 1,
wherein the plurality of pieces of lumen specification information include third lumen specification information,
the processor is configured to output the third lumen specification information in a case in which the second medical image is an image that does not satisfy an erroneous specification triggering condition that triggers erroneous specification of the second existence position by the trained model, and
the third lumen specification information is generated based on information obtained from the trained model in a case in which the second medical image is input to the trained model, and is information capable of specifying a third existence position that is the existence position of the lumen, which is shown in the second medical image, in the second medical image.

5. The medical support device according to claim 2,
wherein the erroneous specification triggering condition includes
a first condition in which at least a part of the lumen is not shown in the second medical image,
a second condition in which at least one dark portion different from the lumen is shown in the second medical image,
a third condition in which an obstruction that obstructs the lumen is shown in the second medical image,
a fourth condition in which an image quality triggers the erroneous specification, and/or
a fifth condition in which a portion of the luminal organ that triggers the erroneous specification is shown in the second medical image.

6. The medical support device according to claim 2,
wherein the trained model generates a confidence level indicating that the lumen is shown for each of a plurality of divided regions obtained by dividing the second medical image or an image corresponding to the second medical image, in a case in which the second medical image is input to the trained model, and
the erroneous specification triggering condition includes a confidence level condition related to reliability of the confidence level for each of the plurality of divided regions.

7. The medical support device according to claim 6,
wherein the confidence level condition includes a condition in which the confidence level for each of the plurality of divided regions is equal to or greater than a second threshold value, which is less than a first threshold value, and less than the first threshold value.

8. The medical support device according to claim 1,
wherein the first medical image or an image corresponding to the first medical image has a plurality of partition regions obtained by partitioning the first medical image or the image corresponding to the first medical image along a circumferential direction,
the trained model generates a plurality of confidence levels corresponding to the plurality of partition regions and indicating that the lumen is shown for each of the plurality of partition regions, in a case in which the first medical image or the image corresponding to the first medical image is input to the trained model,
the first lumen specification information is information capable of specifying the first existence position more precisely than the partition regions in the first medical image or the image corresponding to the first medical image, and
the first lumen specification information is generated based on the plurality of partition regions and the plurality of confidence levels.

9. The medical support device according to claim 8,
wherein the first lumen specification information is information capable of specifying the first existence position with a higher resolution than the plurality of partition regions along the circumferential direction.

10. The medical support device according to claim 8,
wherein a direction from a reference position of the first medical image or the image corresponding to the first medical image to an existence position of each of the plurality of partition regions is determined by a plurality of first vectors,
a direction from the reference position to a lumen existence region including the first existence position is determined by a second vector,
the second vector is a sum of at least two third vectors obtained by adding the confidence level, as a weight, to at least two first vectors among the plurality of first vectors, and
the first lumen specification information is information determined based on the second vector.

11. The medical support device according to claim 1,
wherein the second medical image or an image corresponding to the second medical image has a central region of the second medical image and a plurality of partition regions obtained by radially partitioning surroundings of the central region in the second medical image,
the trained model generates a confidence level indicating that the lumen is shown for the central region and each of the plurality of partition regions, in a case in which the second medical image is input to the trained model, and
in a case in which the confidence level for each of the central region and the plurality of partition regions is equal to or greater than a third threshold value or the confidence level for each of the central region and the plurality of partition regions is less than a fourth threshold value less than the third threshold value, information based on a processing result obtained by the trained model is output preferentially over information based on a processing result obtained by the time-series model.

12. The medical support device according to claim 1,
wherein the trained model generates a confidence level indicating that the lumen is shown in the first medical image, in a case in which the first medical image is input to the trained model, and
in a case in which the confidence level is equal to or less than a reference value, the output of the lumen specification information, which is output before the confidence level equal to or less than the reference value is generated, is continued.

13. The medical support device according to claim 12,
wherein, in a case in which a state in which the confidence level generated by the trained model is equal to or less than the reference value continues for a predetermined period each time the first medical image is input to the trained model, an output level of the lumen specification information, which is output before the predetermined period starts, gradually decreases throughout the predetermined period.

14. The medical support device according to claim 1,
wherein the output of the lumen specification information is carried out by displaying the lumen specification information on a screen.

15. The medical support device according to claim 14,
wherein the medical image and/or an image corresponding to the medical image is displayed on the screen, and
the lumen specification information is displayed on the screen in a state of being comparable with the medical image or the image corresponding to the medical image displayed on the screen.

16. The medical support device according to claim 15,
wherein the lumen specification information is displayed in a superimposed manner on the medical image and/or the image corresponding to the medical image.

17. The medical support device according to claim 15,
wherein the lumen specification information displayed on the screen is updated in accordance with the display of the medical image and/or the image corresponding to the medical image.

18. An endoscope system comprising:
the medical support device according to claim 1; and
an endoscope,
wherein the medical image is generated by imaging the inside of the luminal organ with the endoscope.

19. A medical support method comprising:
acquiring a medical image generated by imaging an inside of a luminal organ; and
selectively outputting a plurality of pieces of lumen specification information that are information capable of specifying an existence position of a lumen, which is shown in the medical image, in the medical image,
wherein the medical image is classified into a first medical image and a second medical image obtained later than the first medical image,
the plurality of pieces of lumen specification information include first lumen specification information and second lumen specification information,
the first lumen specification information is generated based on information obtained from a trained model in a case in which the first medical image is input to the trained model, and is information capable of specifying a first existence position that is the existence position of the lumen, which is shown in the first medical image, in the first medical image, and
the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image.

20. A non-transitory computer-readable storage medium storing a program executable by a computer to execute medical support processing comprising:
acquiring a medical image generated by imaging an inside of a luminal organ; and
selectively outputting a plurality of pieces of lumen specification information that are information capable of specifying an existence position of a lumen, which is shown in the medical image, in the medical image,
wherein the medical image is classified into a first medical image and a second medical image obtained later than the first medical image,
the plurality of pieces of lumen specification information include first lumen specification information and second lumen specification information,
the first lumen specification information is generated based on information obtained from a trained model in a case in which the first medical image is input to the trained model, and is information capable of specifying a first existence position that is the existence position of the lumen, which is shown in the first medical image, in the first medical image, and
the second lumen specification information is generated based on information obtained from a time-series model in a case in which time-series information that is information related to one or more pieces of the first lumen specification information obtained in time series is input to the time-series model, and is information capable of specifying a second existence position that is the existence position of the lumen, which is shown in the second medical image, in the second medical image.
